(19) United States
(12) Patent Application Publication
(10) Pub. No.: US 2022/0089663 A1
(43) Pub. Date: Mar. 24, 2022

(54) PROTEINS AND FLUOROPHORE-CONTAINING COMPOUNDS SELECTIVE FOR NaV1.7

(71) Applicants: MEMORIAL SLOAN KETTERING CANCER CENTER, New York, NY (US); THE UNIVERSITY OF QUEENSLAND, Brisbane (AU)

(72) Inventors: Paula Demetrio DE SOUZA FRANCA, New York, NY (US); Junior GONZALES, New York, NY (US); Jason LEWIS, New York, NY (US); Thomas REINER, New York, NY (US); Glenn F. KING, Brisbane (AU)

(21) Appl. No.: 17/423,341

(22) PCT Filed: Jan. 17, 2020
(86) PCT No.: PCT/US2020/014212

§ 371 Date: Jul. 15, 2021

Related U.S. Application Data
(60) Provisional application No. 62/794,520, filed on Jan. 18, 2019.
(60) Provisional application No. 62/873,652, filed on Jul. 12, 2019.

(51) Int. Cl. C07K 14/435; C07K 14/705

(52) U.S. Cl. CPC C07K 14/43518; G01N 21/6428; C07K 14/705

(57) ABSTRACT

The present technology is directed to fluorophore-containing compounds useful in the imaging of peripheral neurons as well as to proteins useful in the treatment (including management) of pain.

### CROSS-REFERENCE TO RELATED APPLICATIONS

This application is a U.S. National Phase Application under 35 U.S.C. § 371 of International Application No. PCT/US2020/014212, filed Jan. 17, 2020, which claims the benefit of and priority to U.S. Provisional Application 62/794,520, filed Jan. 18, 2019, and U.S. Provisional Application 62/873,652, filed Jul. 12, 2019, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on Feb. 27, 2020, is named 115872-0588_SL.txt and is 13,646 bytes in size.

### FIELD

The present technology is directed to fluorophore-containing compounds useful in the imaging of peripheral neurons as well as to proteins useful in the treatment (including management) of pain.

### SUMMARY

In an aspect, the present technology provides a compound of a fluorophore conjugated to a side chain of an amino acid of a peptide of SEQ ID NO: 1 (YCQKFLWTCDSERPCCEGLVCRLWCKIN-NH₂), or a conservative amino acid substitution variant thereof, a pharmaceutically acceptable salt thereof, and/or a solvate thereof.

In another aspect, the present technology provides a compound of a fluorophore conjugated to a side chain of an amino acid of a peptide of SEQ ID NO: 2 (GNDCLGFWSACNPKNDKCCANLVCSSKHKWCKGKL-NH₂), or a conservative amino acid substitution variant thereof, a pharmaceutically acceptable salt thereof, and/or a solvate thereof.

In another aspect, the present technology provides a protein of SEQ ID NO: 1 (YCQKFLWTCDSERPCCEGLVCRLWCKIN-NH₂), or a conservative amino acid substitution variant thereof, a pharmaceutically acceptable salt thereof, and/or a solvate thereof. The protein may be an isolated protein. The proteins of the present technology are useful in treating pain in a subject while avoiding the deleterious side effects typically elicited by analgesics.

In an aspect, the present technology also provides compositions that include any aspect or embodiment of a compound of the present technology as disclosed herein and a pharmaceutically acceptable carrier or include any embodiment disclosed herein of a protein of the present technology and a pharmaceutically acceptable carrier. In a related aspect, the present technology provides pharmaceutical compositions that include an effective amount of a compound of the present technology disclosed herein and a pharmaceutically acceptable carrier or include an effective amount of a protein of the present technology disclosed herein and a pharmaceutically acceptable carrier. Further aspects are directed to methods of use of a compound of the present technology or a protein of the present technology, including methods of treatment by administration of a compound of the present technology.

### BRIEF DESCRIPTION OF THE DRAWINGS

FIG. 1A shows concentration-response curves of the active fractions of the crude Homoeomma spec. Peru venom on the hNaᵥ1.1 (black) and 1.6 (green) sodium channels and that Fraction 22 (F22) showed the selectivity of hNaᵥ1.1 to hNaᵥ1.6. The concentration was calculated as the initial venom equivalent. Fitting the log (inhibitor) vs. response (three parameters) in prism 7 (n=1).

FIG. 1B shows sequence alignment of Hsp1a, isolated from the active fractions of the crude Homoeomma spec. Peru venom, with known spider venom peptides, and that Hsp1a exhibited at least 70% sequence identity to known spider venom peptides. Identical residues are indicated in bold and the C-terminal amidated peptides are labelled with asterisk. FIG. 1B discloses SEQ ID NOS 1 and 14-30, respectively, in order of appearance.

FIG. 1C shows the concentration-response curves of native Hsp1a (blue), sHsp1a (red) and rHsp1a (green) on the hNaᵥ1.7 channel. Fitting the log (inhibitor) vs. response (three parameters) in prism 7 (n≥5; mean±SEM).

FIG. 1D shows a bar graph comparing the pIC₅₀s of Hsp1a, sHsp1a and rHsp1a (n≥5; mean±SEM).

FIG. 1E shows representative traces of hNaᵥ currents without (black) and with the presence of 200 nM native Hsp1a (blue), 200 nM of sHsp1a (red) and rHsp1a (green) respectively.

FIG. 2A shows the amino acid sequences and primary structures of sHsp1a, Hsp1a and rHsp1a, including the additional serine residue. FIG. 2A discloses SEQ ID NOS 31, 1 and 32, respectively, in order of appearance.

FIG. 2B shows representative RP-HPLC chromatograph traces demonstrating that the Co-elution of native Hsp1a (black trace), sHsp1a (red trace), rHsp1a (green trace) and the mixture of sHsp1a and native Hsp1a (blue trace) from an analytical PEPTIDE XB-C18 column.

FIG. 3A shows concentration-response curves of sHsp1a on hNaᵥ1.1-1.7 illustrating the inhibitory effect of sHsp1a on the channel. Fitting the log (inhibitor) vs. response (three parameters) in prism 7 (n≥5; mean±SEM).

FIG. 3B shows a bar graph demonstrating that the pIC₅₀s of sHsp1a on hNaᵥ1.7 was 40 fold selective over hNaᵥ1.1 and 28 fold selective over hNaᵥ1.2 (n≥5; mean±SEM).

FIG. 3C shows representative traces of hNaᵥ1.1, hNaᵥ1.2, hNaᵥ1.3, hNaᵥ1.4, hNaᵥ1.5, hNaᵥ1.6, and hNaᵥ1.7 currents in the absence (black) or the presence of 200 nM of sHsp1a (red).

FIG. 4A shows a graph illustrating the conductance-voltage (G-V) relationship of the hNaᵥ1.7 channel before (black) and after (red) addition of 60 nM sHsp1a. Currents were evoked from a holding potential of −120 mV, stepping from −80 to +60 mV in 5 mV increments. The V0.5 activation was calculated as −16.03±0.23 mV in the absent and −12.00±2.28 mV in the present of 60 nM sHsp1a by fitting Boltzmann equation in prism 7 (n=5; mean±SEM).

FIG. 4B shows a graph illustrating the steady-state inactivation of the hNaᵥ1.7 channel before (black) and after (red) addition of 60 nM sHsp1a. Currents were evoked by a depolarization to 0 mV following a series step potential from ˜120 mV to 20 mV in 5 mV increments. The V0.5 inactivation was calculated as −67.56±0.98 mV in the absent and −79.18±1.07 mV in the present of 60 nM sHsp1a by fitting Boltzmann equation in prism 7 (n=5; mean±SEM).

FIG. 4C shows a graph comparing V_{0.5} of activation and inactivation of the hNaᵥ1.7 channel in the absent (grey) and present (red) of 60 nM sHsp1a (n=5; mean±SEM).

FIG. 4D shows the hNaᵥ1.7 channel recovery from fast inactivation before (black) and after (red) addition of 60 nM sHsp1a. Currents were evoked from a holding potential of −120 mV by applying a 0 mV pulse followed by repolarisation to −120 mV. A second pulse to 0 mV was applied after a period ranging from 0 to 50 ms, increasing in 1 ms increments. The time constant τ was calculated as 4.05±0.34 ms in the absent and 5.03±0.43 ms in the present of 60 nM sHsp1a by fitting one-phase decay in prism 7 (n=5; mean±SEM).

FIG. 4E shows a physiological trace of hNaᵥ1.7 currents (I/Imax) following application of 200 nM sHsp1a for 4 min followed by washout for 12 min. Depolarizing pulses were applied from ˜120 to 0 mV every 6 s and the holding potential was −80 mV.

FIG. 5A shows the stability of sHsp1a in plasma at 37° C. when compared to ω-conotoxin MVIIA (MVIIA) and human Atrial Natriuretic Peptide (hANP) during the first hour of incubation.

FIG. 5B shows the stability of sHsp1a in plasma at 37° C. 2-24 hours after incubation when compared to ω-conotoxin MVIIA (MVIIA) and human Atrial Natriuretic Peptide (hANP).

FIG. 6A shows raw traces illustrating the visceromotor response (VMR) to colorectal distension (CRD) in I) healthy mice treated with vehicle II) chronic visceral hypersensitivity mice (CVH) treated with vehicle and III) CVH mice treated with sHsp1a (200 nM).

FIG. 6B shows a graph illustrating that the Visceromotor response (VMR) to colorectal distension (CRD) was significantly increased in the chronic visceral hypersensitivity animal model (CVH+Veh, N=10 mice) when compared to healthy control animals (HC+Veh, N=10 mice) (*p<0.05 at 50 mmHg; **p<0.01 at 70 mmHg and ***p<0.001 at 60 and 80 mmHg); and that the Intra-colonic administration of sHsp1a (200 nM) in CVH mice (CVH+sHsp1a, N=8 0 mice) significantly reduced the VMR to colorectal distension when compared to CVH vehicle treated animals normalizing the responses to healthy levels.

FIG. 6C shows that colonic compliance was not significantly altered by intra-colonic sHsp1a administration relative to intra-colonic vehicle administration (*, P<0.05 at 80 mmHg).

FIG. 7A shows a graph illustrating that sHsp1a had no effect on the respiratory musculature as demonstrated by oxygen saturation in the peripheral blood before, 15 seconds, 5 minutes and 15 minutes after injection with sHsp1a.

FIG. 7B shows a graph illustrating that sHsp1a had no effect on heart muscle as demonstrated by mice heart rate before, 15 seconds, 5 minutes and 15 minutes after injection with sHsp1a.

FIG. 7C shows representative electrocardiogram traces illustrating that sHsp1a had no effect on heart rate under the same experimental conditions as FIG. 7B.

FIG. 7D shows a graph illustrating that sHsp1a had no effect on mouse body temperature.

FIG. 8A shows a table illustrating the nucleophilic modification of Hsp1a and highlighting lysine K4 where the dyes primarily attached, and major ion species obtained by LC-MS that confirmed Hsp1a conjugation to the dye. FIG. 8A discloses SEQ ID NOS 1 and 5-10, respectively, in order of appearance.

FIG. 8B shows concentration-response curves illustrating that the inhibitory effect of Hsp1a-FL on the hNaᵥ1.7 was attenuated (IC₅₀=62 nM), but Hsp1a-FL remained as potent as Hsp1a ((IC₅₀=13 nM). Data were fit in prism 8 (n>5; mean±SEM).

FIG. 8C shows representative hNaᵥ1.7 current traces in the absence (solid) or presence of 2 μM Hsp1a-FL or Hsp1a (dashed) illustrating that Hsp1a-FL is a potent inhibitor of the hNaᵥ1.7 channel.

FIG. 9A shows an epifluorescence images illustrating the accumulation of Hsp1a-FL in fresh, unprocessed mouse sciatic nerves 30 minutes after systemic injection. In particular, the figure shows the inhibitory effect of unlabeled Hsp1a (Hsp1a-FL, 68 μM, 7 nmol and Hsp1a, 204 μM, 21 nmol in 100 μL PBS) on the Hsp1a-FL.

FIG. 9B shows an epifluorescence images illustrating the accumulation of Hsp1a-FL in resected right and left mouse sciatic nerves 30 minutes after systemic injection under the same conditions as FIG. 9A.

FIG. 9C shows a bar graph quantifying the accumulation Hsp1a-FL in resected right and left mouse sciatic nerves of FIG. 9B.

FIG. 9D shows an epifluorescence images illustrating the accumulation of Hsp1a-FL in fresh, unprocessed mouse sciatic nerves 30 minutes after systemic injection as discussed in FIG. 9A.

FIG. 9E shows an epifluorescence images illustrating the accumulation of Hsp1a-FL in resected right and left mouse sciatic nerves 30 minutes after systemic injection as discussed in FIG. 9B.

FIG. 9F shows a bar graph quantifying the biodistribution of Hsp1a-FL in various tissues and demonstrating that Hsp1a-FL effect is limited to the peripheral nervous system.

FIG. 10 show an immunofluorescence image demonstrating that Hsp1a-FL is expressed in mouse sciatic nerve, but not in the muscle or brain.

FIG. 11A shows an unprocessed epifluorescence images illustrating the accumulation of Hsp1a-IR800 in fresh mouse sciatic nerves 30 minutes after systemic injection under similar conditions as FIG. 9A.

FIG. 11B shows a bar graph quantifying the accumulation Hsp1a-IR800 in fresh mouse sciatic nerves of FIG. 11A.

FIG. 11C shows a processed epifluorescence images illustrating the accumulation of Hsp1a-IR800 in fresh mouse sciatic nerves 30 minutes after systemic injection under similar conditions as FIG. 11A.

FIG. 11D shows a bar graph quantifying the accumulation Hsp1a-IR800 in fresh mouse sciatic nerves of FIG. 11C.

FIG. 12A shows process and unprocessed epifluorescence images illustrating the accumulation of Hsp1a-IR800 in resected right and left mouse sciatic nerves 30 minutes after systemic injection as discussed in FIG. 9B.

FIG. 12B shows a bar graph quantifying the accumulation Hsp1a-IR800 in fresh mouse sciatic nerves of FIG. 12A.

FIG. 12C shows process and unprocessed epifluorescence images illustrating the accumulation of Hsp1a-IR800 in various tissues and demonstrating that Hsp1a-IR800 effect is limited to the peripheral nervous system.

FIG. 12D shows a bar graph quantifying the biodistribution of Hsp1a-IR800 in the tissues shown in FIG. 12C.

FIG. 13A shows an unprocessed epifluorescence images illustrating the accumulation of Hsp1a-DY-684 in fresh mouse sciatic nerves 30 minutes after systemic injection under similar conditions as FIG. 9A.

FIG. 13B shows a bar graph quantifying the accumulation Hsp1a-DY-684 in fresh mouse sciatic nerves of FIG. 13A.

FIG. 13C shows a processed epifluorescence images illustrating the accumulation of Hsp1a-DY-684 in fresh mouse sciatic nerves 30 minutes after systemic injection under similar conditions as FIG. 13A.

FIG. 13D shows a bar graph quantifying the accumulation Hsp1a-DY-684 in fresh mouse sciatic nerves of FIG. 13C.

FIG. 14A shows process and unprocessed epifluorescence images illustrating the accumulation of Hsp1a-DY-684 in resected right and left mouse sciatic nerves 30 minutes after systemic injection as discussed in FIG. 9B.

FIG. 14B shows a bar graph quantifying the accumulation Hsp1a-DY-684 in fresh mouse sciatic nerves of FIG. 14A.

FIG. 14C shows process and unprocessed epifluorescence images illustrating the accumulation of Hsp1a-DY-684 in various tissues and demonstrating that Hsp1a-DY-684 effect is limited to the peripheral nervous system.

FIG. 14D shows a bar graph quantifying the biodistribution of Hsp1a-DY-684 in the tissues shown in FIG. 14C.

FIG. 15A shows an unprocessed epifluorescence images illustrating the accumulation of Hsp1a-Janelia669 in fresh mouse sciatic nerves 30 minutes after systemic injection under similar conditions as FIG. 9A.

FIG. 15B shows a bar graph quantifying the accumulation Hsp1a-Janelia669 in fresh mouse sciatic nerves of FIG. 15A.

FIG. 15C shows a processed epifluorescence images illustrating the accumulation of Hsp1a-Janelia669 in fresh mouse sciatic nerves 30 minutes after systemic injection under similar conditions as FIG. 15A.

FIG. 15D shows a bar graph quantifying the accumulation Hsp1a-Janelia669 in fresh mouse sciatic nerves of FIG. 15C.

FIG. 16A shows process and unprocessed epifluorescence images illustrating the accumulation of Hsp1a-Janelia669 in resected right and left mouse sciatic nerves 30 minutes after systemic injection as discussed in FIG. 9B.

FIG. 16B shows a bar graph quantifying the accumulation Hsp1a-Janelia669 in fresh mouse sciatic nerves of FIG. 16A.

FIG. 16C shows process and unprocessed epifluorescence images illustrating the accumulation of Hsp1a-Janelia669 in various tissues and demonstrating that Hsp1a-Janelia669 effect is limited to the peripheral nervous system.

FIG. 16D shows a bar graph quantifying the biodistribution of Hsp1a-Janelia669 in the tissues shown in FIG. 16C.

FIG. 17A shows an unprocessed epifluorescence images illustrating the accumulation of Hsp1a-BODIPY665 in fresh mouse sciatic nerves 30 minutes after systemic injection under similar conditions as FIG. 9A.

FIG. 17B shows a bar graph quantifying the accumulation Hsp1a-BODIPY665 in fresh mouse sciatic nerves of FIG. 17A.

FIG. 17C shows a processed epifluorescence images illustrating the accumulation of Hsp1a-BODIPY665 in fresh mouse sciatic nerves 30 minutes after systemic injection under similar conditions as FIG. 17A.

FIG. 17D shows a bar graph quantifying the accumulation Hsp1a-BODIPY665 in fresh mouse sciatic nerves of FIG. 17C.

FIG. 18A shows process and unprocessed epifluorescence images illustrating the accumulation of Hsp1a-IR800 in resected right and left mouse sciatic nerves 30 minutes after systemic injection as discussed in FIG. 9B.

FIG. 18B shows a bar graph quantifying the accumulation Hsp1a-BODIPY665 in fresh mouse sciatic nerves of FIG. 18A.

FIG. 18C shows process and unprocessed epifluorescence images illustrating the accumulation of Hsp1a-BODIPY665 in various tissues and demonstrating that Hsp1a-BODIPY665 effect is limited to the peripheral nervous system.

FIG. 18D shows a bar graph quantifying the biodistribution of Hsp1a-BODIPY665 in the tissues shown in FIG. 18C.

FIG. 19A shows an unprocessed epifluorescence images illustrating the accumulation of Hsp1a-CY7.5 in fresh mouse sciatic nerves 30 minutes after systemic injection under similar conditions as FIG. 9A.

FIG. 19B shows a bar graph quantifying the accumulation Hsp1a-CY7.5 in fresh mouse sciatic nerves of FIG. 19A.

FIG. 19C shows a processed epifluorescence images illustrating the accumulation of Hsp1a-CY7.5 in fresh mouse sciatic nerves 30 minutes after systemic injection under similar conditions as FIG. 19A.

FIG. 19D shows a bar graph quantifying the accumulation Hsp1a-CY7.5 in fresh mouse sciatic nerves of FIG. 19C.

FIG. 20A shows process and unprocessed epifluorescence images illustrating the accumulation of Hsp1a-IR800 in resected right and left mouse sciatic nerves 30 minutes after systemic injection as discussed in FIG. 9B.

FIG. 20B shows a bar graph quantifying the accumulation Hsp1a-CY7.5 in fresh mouse sciatic nerves of FIG. 20A.

FIG. 20C shows process and unprocessed epifluorescence images illustrating the accumulation of Hsp1a-CY7.5 in various tissues and demonstrating that Hsp1a-CY7.5 effect is limited to the peripheral nervous system.

FIG. 20D shows a bar graph quantifying the biodistribution of Hsp1a-CY7.5 in the tissues shown in FIG. 20C.

FIG. 21A shows an immunofluorescence image of exposed mouse sciatic nerves acquired by a Lumar surgical fluorescence stereoscope confirming the feasibility of using Hsp1a clinically as a biomarker for intraoperative contemporaneous mapping of peripheral nerves. The sciatic nerves were prepared as discussed in FIG. 9A.

FIG. 21B shows an immunofluorescence image of resected right and left mouse sciatic nerves 30 minutes after systemic injection acquired by a Lumar surgical fluorescence stereoscope.

FIG. 21C shows a bar graph quantifying the fluorescence signal of FIG. 21B.

FIG. 21D shows a high magnification of a resected sciatic nerve from a mouse injected with Hsp1a-FL illustrating tubular (left) and axonal (right) features labeled by Hsp1a-FL 30 minutes post-injection.

FIG. 22A shows a graph illustrating the selectivity of Hs1a-FL towards human Naᵥ channels stably expressed in HEK293 cells. In particular, Hs1a-FL inhibited hNaᵥ1.1 (IC₅₀=19.4 nM), hNaᵥ1.2 (IC₅₀=81.2 nM), hNaᵥ1.3 (IC₅₀=106.8 nM), hNaᵥ1.6 (IC₅₀=19.2 nM), and, hNaᵥ1.7 (IC₅₀=26.9 nM), but not hNaᵥ1.4 (IC₅₀>3000 nM) and hNaᵥ1.5 (IC₅₀>3000 nM).

FIG. 22B shows a bar graph quantifying the fluorescence generated by the accumulation of Hs1a-FL in mouse sciatic nerve from mice injected with PBS, Hs1a-FL or a combination (Hs1a-FL, 45 μM, 4 nmol and Hs1a 120 μM, 12 nmol in 100 μL PBS).

FIG. 23A shows an epifluorescence images illustrating the accumulation of Hs1a-FL in fresh, unprocessed mouse sciatic nerves 30 minutes after systemic injection. In particular, the figure shows the inhibitory effect of unlabeled Hs1a (Hs1a-FL, 45 μM, 4 nmol and Hs1a 120 μM, 12 nmol in 100 μL PBS).

FIG. 23B shows a bar graph quantifying the florescence intensity of FIG. 23A.

FIG. 23C shows a RP-HPLC chromatogram of Hs1a-FL (left), microscopy image from mouse sciatic nerve, IgG control (middle), and an epifluorescence images illustrating the accumulation of Hs1a-FL in resected right and left mouse sciatic nerves 30 minutes after systemic injection (right) as discussed in FIG. 23A.

FIG. 24A shows epifluorescence images illustrating the accumulation of Hs1a-FL in various tissues 30 minutes post-injection under the conditions discussed in FIG. 23A.

FIG. 24B shows a bar graph quantifying the florescence intensity of Hs1a-FL in the tissues shown in FIG. 24A.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, or a hydroxyl group(s) it can form salts with metals, such as alkali and earth alkali metals (e.g. Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms. The phrase “and/or” as used in this paragraph and the present disclosure will be understood to mean any one of the recited members individually or a combination of any two or more thereof—for example, “A, B, and/or C” would mean “A, B, C, A and B, A and C, or B and C.”

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

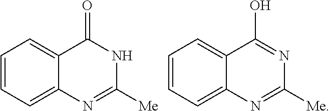

As another example, guanidines may exhibit the following isomeric forms in protic organic solution, also referred to as tautomers of each other:

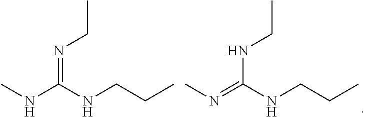

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

As used herein, the terms “subject,” “individual,” or “patient” can be an individual organism, a vertebrate, a mammal, or a human. “Mammal” includes a human, non-human primate, murine (e.g., mouse, rat, guinea pig, hamster), ovine, bovine, ruminant, lagomorph, porcine, caprine, equine, canine, feline, avis, etc. In any embodiment herein, the mammal is feline or canine. In any embodiment herein, the mammal is human.

The term “administering” a compound or composition to a subject means delivering the compound to the subject. “Administering” includes prophylactic administration of the compound or composition (i.e., before the disease and/or one or more symptoms of the disease are detectable) and/or therapeutic administration of the composition (i.e., after the disease and/or one or more symptoms of the disease are detectable). The methods of the present technology include administering one or more compounds or agents. If more than one compound is to be administered, the compounds may be administered together at substantially the same time, and/or administered at different times in any order. Also, the compounds of the present technology may be administered before, concomitantly with, and/or after administration of another type of drug or therapeutic procedure (e.g., surgery).

As used herein, the term “amino acid” is used to refer to any organic molecule that contains at least one amino group and at least one carboxyl group where the at least one amino group is at the a position relative to the carboxyl group, where the amino acid is in the L-configuration. Naturally occurring amino acids include, for example, the twenty most common levorotatory (L) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Naturally-occurring amino acids are those encoded by the genetic code, as well as those amino acids that are later modified, e.g., hydroxyproline, γ-carboxyglutamate, and O-phosphoserine. Amino acids may be referred to herein by either their commonly known three letter symbols or by the one-letter symbols recommended by the IUPAC-IUB Biochemical Nomenclature Commission.

As used herein, the terms “polypeptide,” “polyamino acid,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, “isolated” or “purified” polypeptide, peptide, or protein refers to polypeptide, peptide, or protein that is substantially free of cellular material or other contaminating polypeptides from the cell or tissue source from which the agent is derived, or substantially free from chemical precursors or other chemicals when chemically synthesized. For example, an isolated protein would be free of materials that would interfere with therapeutic uses of the agent. Such interfering materials may include enzymes, hormones and other proteinaceous and nonproteinaceous solutes.

“Treating,” “treat,” “treated,” or “treatment” as used herein covers the treatment of a disease or disorder described herein in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, ameliorating, or slowing progression of one or more symptoms of the disease or disorder. Symptoms may be assessed by methods known in the art or described herein, for example, biopsy, histology, and blood tests to determine relevant enzyme levels, metabolites or circulating antigen or antibody (or other biomarkers), quality of life questionnaires, patient-reported symptom scores, and imaging tests.

“Ameliorate,” “ameliorating,” and the like, as used herein, refer to inhibiting, relieving, eliminating, or slowing progression of one or more symptoms.

As used herein, “prevention,” “prevents,” or “preventing” of a disorder or condition refers to a compound that, in a statistical sample, reduces the occurrence of the disorder, symptom, or condition in the treated sample relative to a control subject, or delays the onset of one or more symptoms of the disorder or condition relative to the control subject.

Throughout this disclosure, various publications, patents and published patent specifications are referenced by an identifying citation. Also within this disclosure are Arabic numerals referring to referenced citations, the full bibliographic details of which are provided subsequent to the Examples section. The disclosures of these publications, patents and published patent specifications are hereby incorporated by reference into the present disclosure to more fully describe the present technology.

### The Present Technology

Injuries to the peripheral nervous system represent a significant concern in surgical practice, and can occur during virtually any type of intervention. While the majority of peripheral nerve injuries occur in the upper limbs and are of traumatic origin, around 25% of patients suffering from neuropathic pain identified surgical intervention as the originating cause. Oncologic surgery in particular carries a considerable risk of peripheral nerve damage because of the distorted physiology around a malignant lesion and the need to achieve complete resection for oncologic control—which, intuitively, increases the likelihood of inadvertent injury.

The present technology provides fluorophore-containing compounds selective for Naᵥ1.7 and proteins both selective and potent for Naᵥ1.7. Naᵥ1.7 is a sodium channel that is expressed on peripheral neurons and which has received a tremendous amount of attention as a target for analgesics. The fluorophore-containing compounds of the present technology are useful in the imaging of peripheral neurons and the proteins of the present technology are useful in the treatment (including management) of pain.

Thus, in an aspect, the present technology provides a compound of a fluorophore conjugated to a side chain of an amino acid of YCQKFLWTCDSERPCCEGLVCRLWCKIN-NH₂ (SEQ ID NO: 1), or a conservative amino acid substitution variant thereof, a pharmaceutically acceptable salt thereof, and/or a solvate thereof. The term “fluorophore conjugated to a side chain” as used herein means that one or more bonds to a hydrogen atom of the side chain of the amino acid are replaced by either (i) a bond to the fluorophore or (ii) a bond to a linker group where the linker group is covalently bonded to the fluorophore. In any embodiment disclosed herein, the linker group may arise from cross-linking of the fluorophore to the side chain. Cross-linking agents can, for example, be obtained from Pierce Biotechnology, Inc., Rockford, Ill. The Pierce Biotechnology, Inc. web-site can provide assistance. By way of further example, cross-linking agents include, but are not limited to, EGS (i.e., ethylene glycol bis[succinimidylsuccinate]), DSS (i.e., disuccinimidyl suberate), DMA (i.e., dimethyl adipimidate. 2HCl), DTSSP (i.e., 3,3′-dithiobis[sulfosuccinimidylpropionate])), DPDPB (i.e., 1,4-di-[3′-(2′-pyridyldithio)-propionamido]butane), BMH (i.e., bis-maleimidohexane), maleimidyl linkers, alkyl halide linkers, succinimidyl linkers, and the platinum cross-linking agents described in U.S. Pat. Nos. 5,580,990; 5,985,566; and 6,133,038 of Kreatech Biotechnology, B.V., Amsterdam, The Netherlands.

A “conservative amino acid substitution variant” will be well understood by one of ordinary skill in the art. One of ordinary skill in the art understands amino acids may be grouped according to their physicochemical characteristics as follows:

(a) Non-polar amino acids: Ala (A) Ser(S) Thr (T) Pro (P) Gly (G) Cys (C);

(b) Acidic amino acids: Asn (N) Asp (D) Glu (E) Gln (Q);

(c) Basic amino acids: His (H) Arg (R) Lys (K);

(d) Hydrophobic amino acids: Met (M) Leu (L) Ile (I) Val (V); and

(e) Aromatic amino acids: Phe (F) Tyr (Y) Trp (W).

Substitutions of an amino acid in a peptide by another amino acid in the same group are referred to as a conservative substitution (and the resulting peptide a “conservative amino acid substitution variant”) and may preserve the physicochemical characteristics of the original peptide. In contrast, substitutions of an amino acid in a peptide by another amino acid in a different group are generally more likely to alter the characteristics of the original peptide.

In any embodiment disclosed herein, the compound may be of Formula I

(I)
(SEQ ID NO: 3)
YCQK(α¹)FLWTCDSERPCCEGLVCRLWCK(α²)IN-NH₂,

or a conservative amino acid substitution variant thereof, a pharmaceutically acceptable salt thereof, and/or a solvate thereof, wherein at least one of α¹ and α² is a fluorophore conjugated to the side chain amine of K and the remaining one of α¹ and α² is H. In any embodiment disclosed herein, the compound of Formula I may be of Formula IA

(IA)
(SEQ ID NO: 4)
YCQK(α¹)FLWTCDSERPCCEGLVCRLWCKIN-NH₂,

or a conservative amino acid substitution variant thereof, a pharmaceutically acceptable salt thereof, and/or a solvate thereof, wherein α¹ is a fluorophore conjugated to the side chain amine of K. To the extent that a person of ordinary skill in the art would not understand what is meant by “α¹ is a fluorophore conjugated to the side chain amine of K,” provided below is an illustration of Formula IA where the K residue is depicted as a structural representation and where the bolded letters are the one-letter symbols recommended by the IUPAC-IUB Biochemical Nomenclature Commission for amino acids (SEQ ID NO: 4 disclosed below):

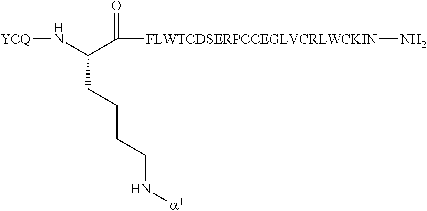

In any embodiment disclosed herein, the fluorophore may independently at each occurrence arises from a fluorescent dye such as IR780, IR800, IR780, DY-684, DY-700, Janelia669, BODIPY, BODIPY665, sulfo-CY5, CY5.5, CY7, CY7.5, ICG, IR780, IR140, or DiR (1,1′-dioctadecyl-3,3,3′,3′-tetramethylindotricarbocyanine). Table A provides an exemplary list of fluorophores that arise from exemplary dyes.

TABLE A

| Fluorophore conjugated to the side chain | Arises From |
|---|---|
| 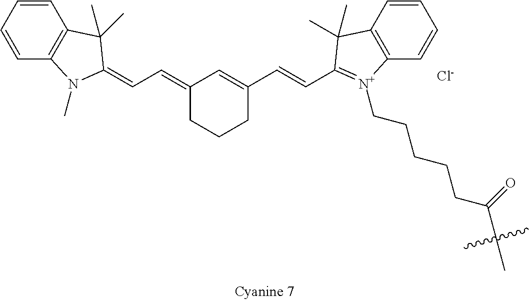 [SMILES: *C(=O)CCCCC[N+]1=C(/C=C/C2=CC(=C/C=C3/N(C)c4ccccc4C3(C)C)/CCC2)C(C)(C)c2ccccc21 ; [Cl-]] | Cy7 (Cyanine 7) |
| 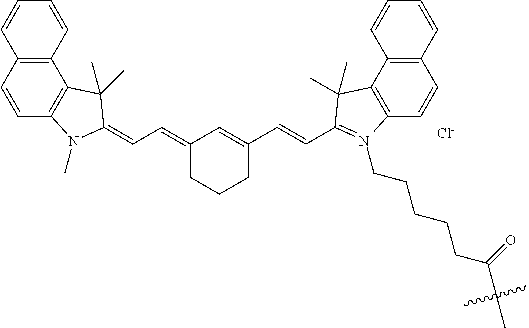 [SMILES: *C(=O)CCCCC[N+]1=C(/C=C/C2=CC(=C/C=C3/N(C)c4ccc5ccccc5c4C3(C)C)/CCC2)C(C)(C)c2c1ccc1ccccc21 ; [Cl-]] | Cy7.5 (Cyanine 7.5) |
| Cyanine 7.5 | |
| 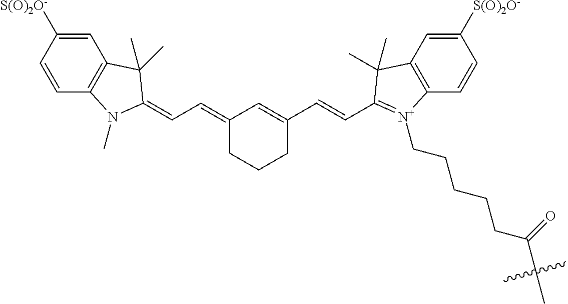 | sulfo-Cy5 |
| 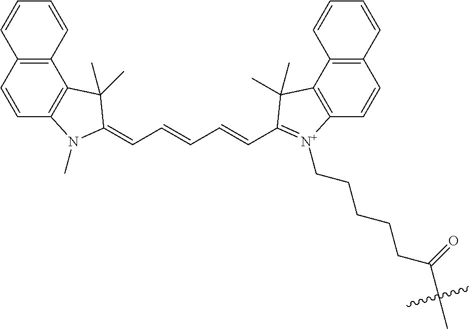 [SMILES: *C(=O)CCCCC[N+]1=C(/C=C/C=C/C=C2/N(C)c3ccc4ccccc4c3C2(C)C)C(C)(C)c2c1ccc1ccccc21] | Cy5.5 |
| 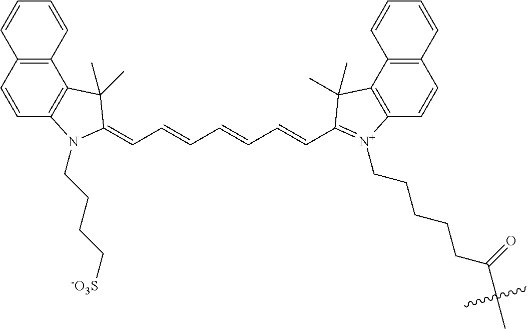 | ICG (Indocyanine green) |
| 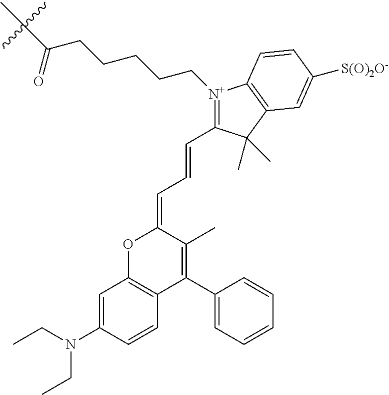 | DY-700 |
| 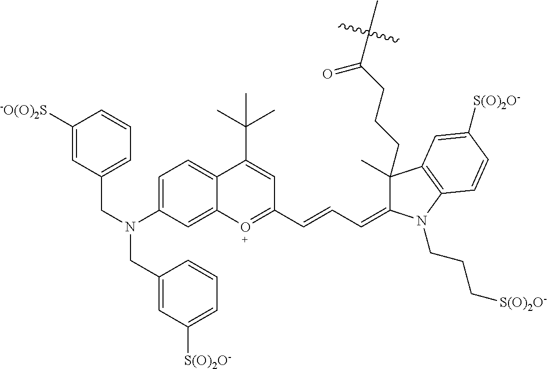 | DY-684 |
| 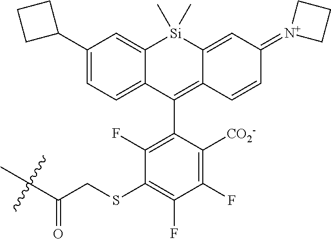 | Janelia669 |
| 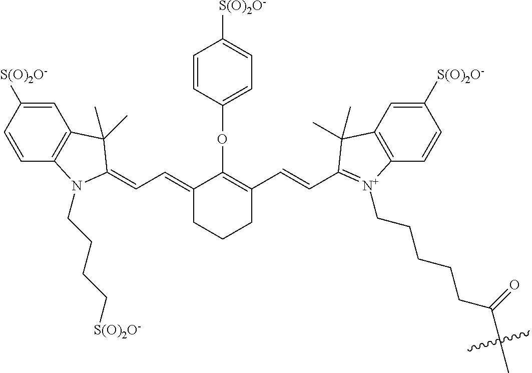 | IR800 |
| 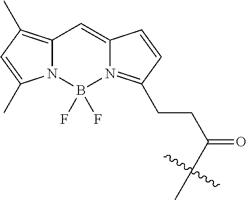 | BODIPY |
| 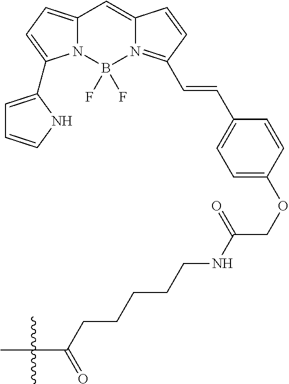 | BODIPY665 |

In any embodiment disclosed herein, it may be the fluorophore is selected from

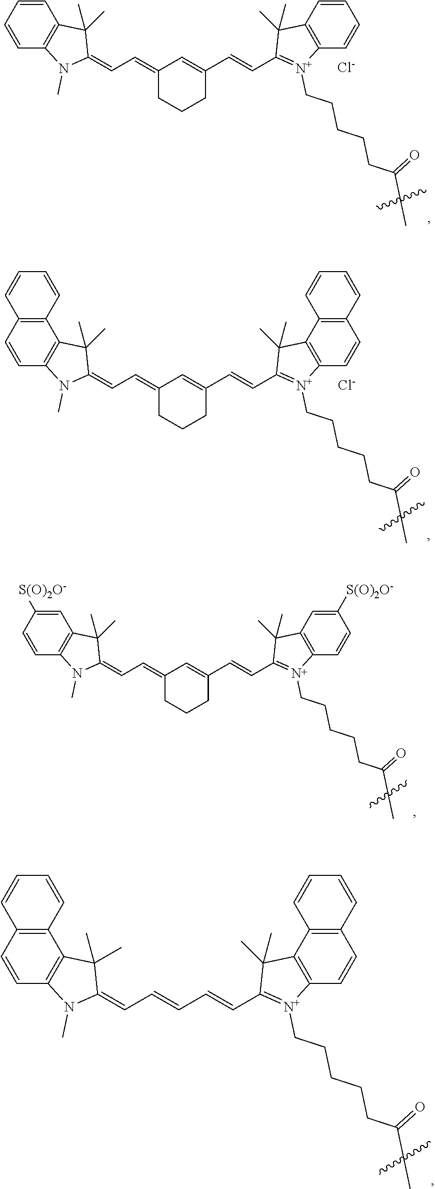

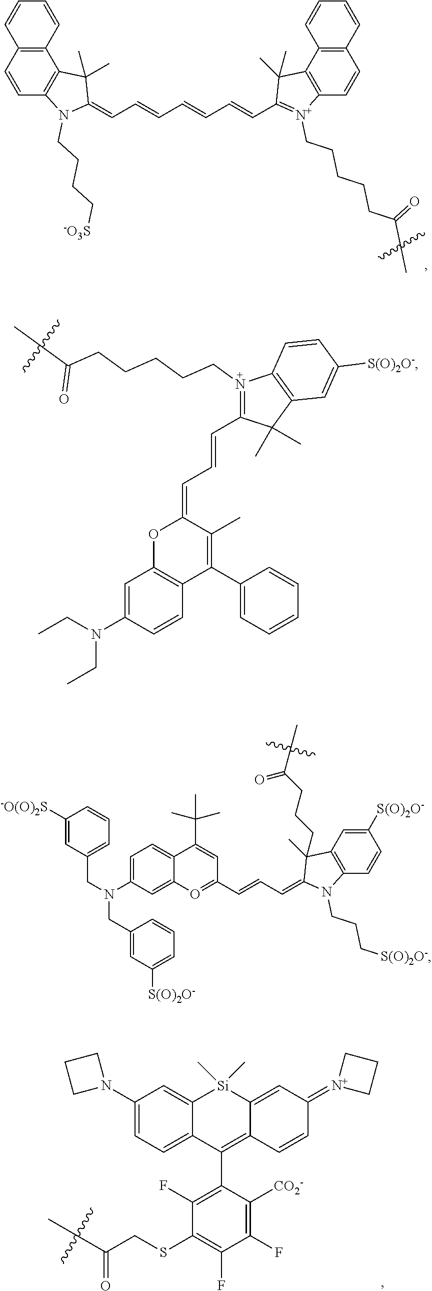

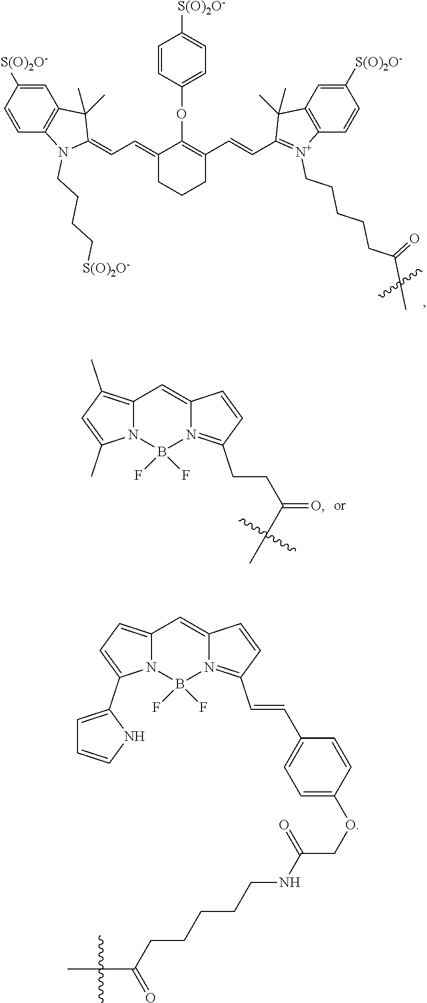

In any embodiment disclosed herein, it may be that the compound is

(SEQ ID NO: 5)
YCQK(BODIPY)FLWTCDSERPCCEGLVCRLWCKIN-NH₂,
(SEQ ID NO: 6)
YCQK(IR-800)FLWTCDSERPCCEGLVCRLWCKIN-NH₂,
(SEQ ID NO: 7)
YCQK(DY-684)FLWTCDSERPCCEGLVCRLWCKIN-NH₂,
(SEQ ID NO: 8)
YCQK(Jane1ia669)FLWTCDSERPCCEGLVCRLWCKIN-NH₂,
(SEQ ID NO: 9)
YCQK(BODIPY665)FLWTCDSERPCCEGLVCRLWCKIN-NH₂,
(SEQ ID NO: 10)
YCQK(CY7.5)FLWTCDSERPCCEGLVCRLWCKIN-NH₂,

or a conservative amino acid substitution variant thereof, a pharmaceutically acceptable salt thereof, and/or a solvate thereof. In any embodiment disclosed herein, the compound may be of Formula IA where α¹ is

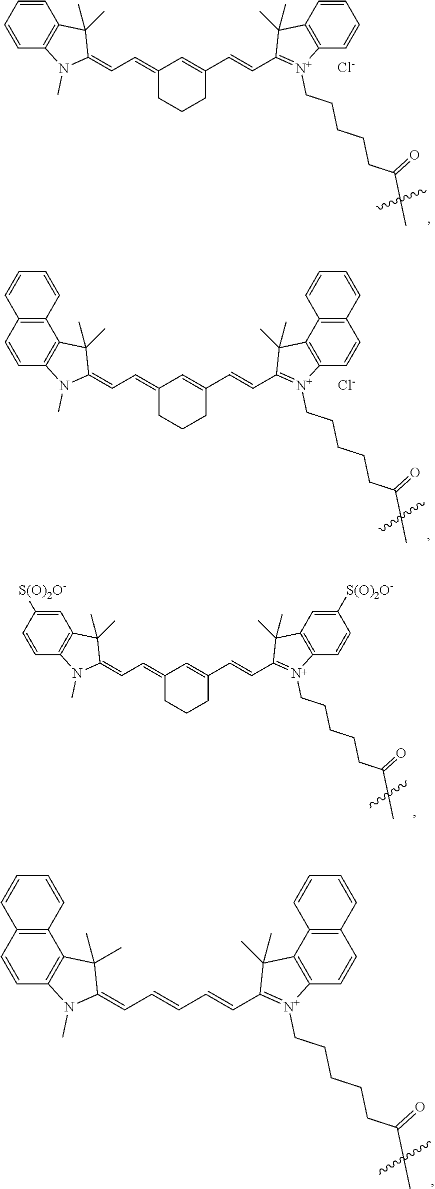

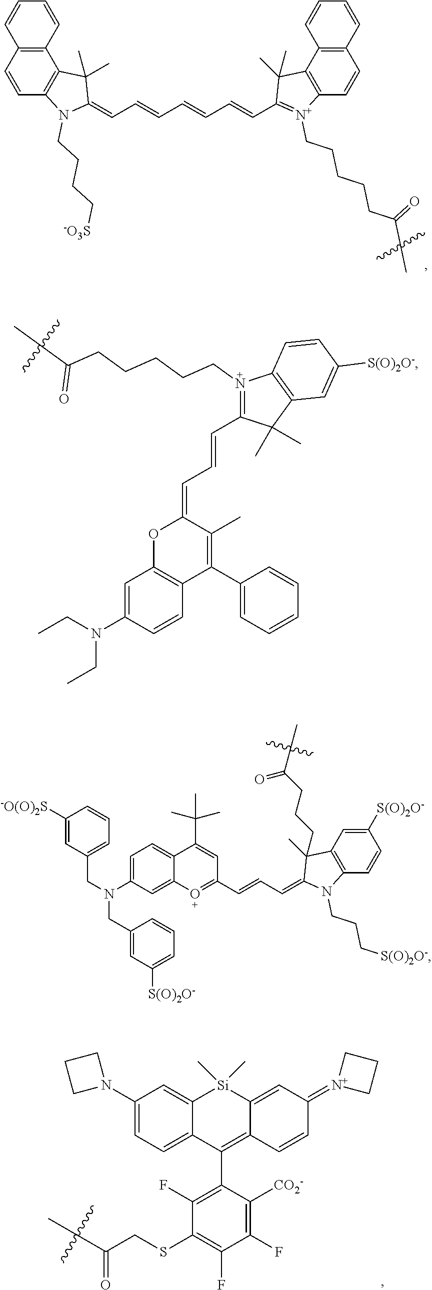

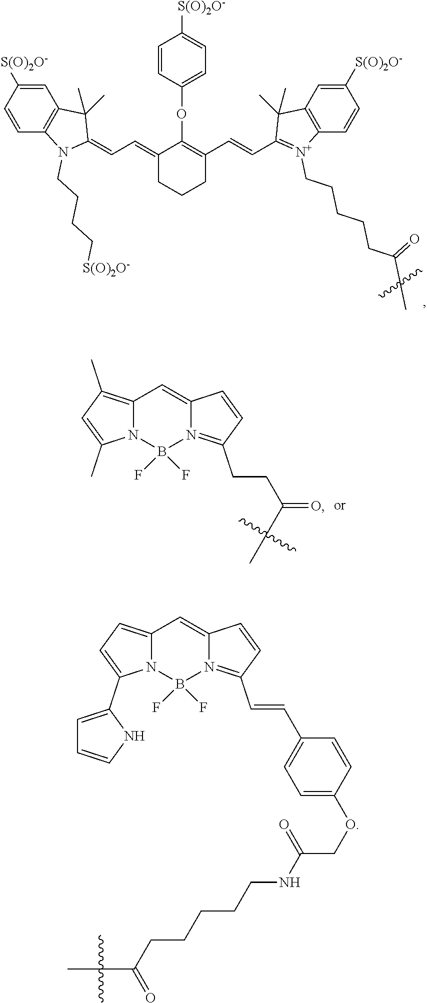

In another aspect, the present technology provides a compound of a fluorophore conjugated to a side chain of an amino acid of GNDCLGFWSACNPKNDKCCANLVCSSKHKWCKGKL-NH₂ (SEQ ID NO: 2), or a conservative amino acid substitution variant thereof, a pharmaceutically acceptable salt thereof, and/or a solvate thereof. In any embodiment herein, the compound may be of Formula II

(II)
(SEQ ID NO: 11)
GNDCLGFWSACNPK(α³)NDK(α⁴)CCANLVCSSK(α⁵)HK(α⁶)WCK(α⁷)G
K(α⁸)L-NH₂

or a conservative amino acid substitution variant thereof, a pharmaceutically acceptable salt thereof, and/or a solvate thereof, wherein at least one of α³, α⁴, α⁵, α⁶, α⁷, and α⁸ is a fluorophore conjugated to the side chain amine of K and the remaining and the remaining of α³, α⁴, α⁵, α⁶, α⁷, and α⁸ are each H. In any embodiment disclosed herein, it may be that α³ is the fluorophore conjugated to the side chain amine of K and α⁴, α⁵, α⁶, α⁷, and α⁸ are each independently H. In any embodiment disclosed herein, it may be that the fluorophore independently at each occurrence arises from IR780, IR800, IR780, DY-684, DY-700, Janelia669, BODIPY, BODIPY665, sulfo-CY5, CY5.5, CY7, CY7.5, ICG, IR780, IR140, or DiR. In any embodiment disclosed herein, it may be the fluorophore is selected from

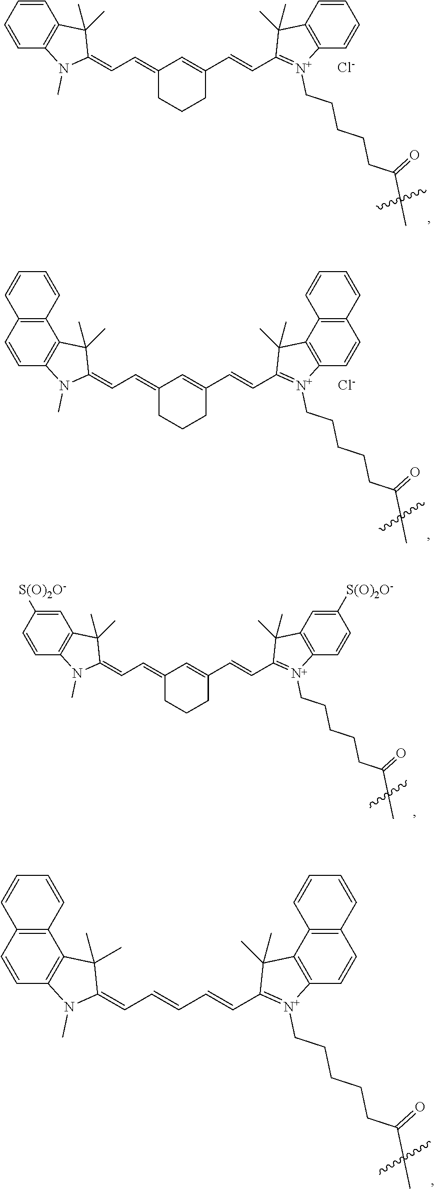

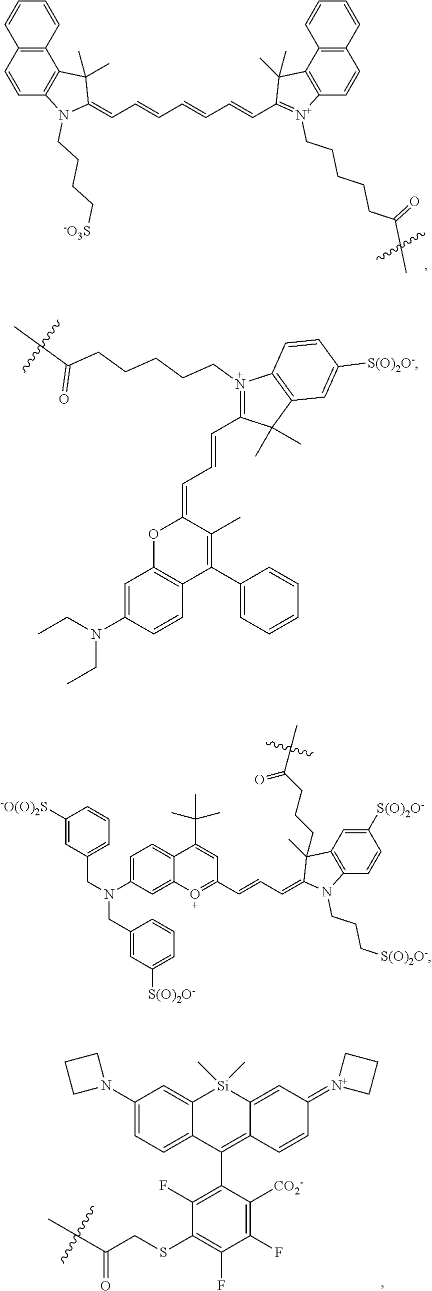

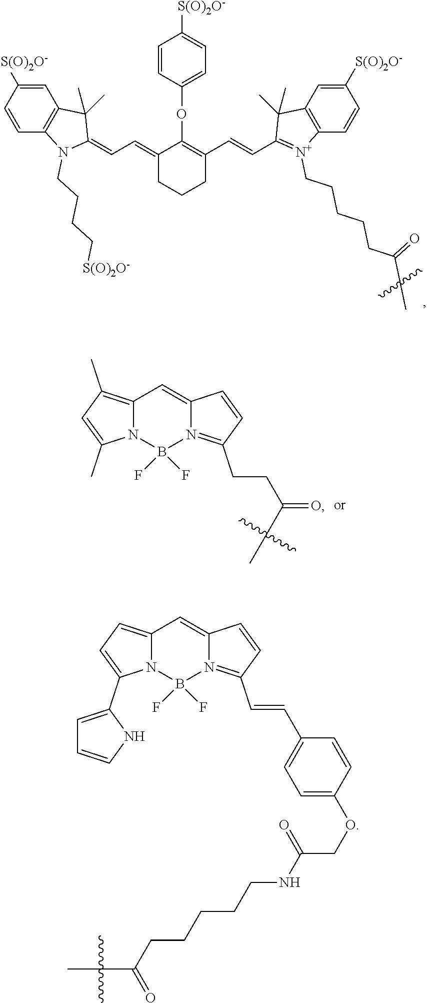

In any embodiment disclosed herein, it may be that the compound is of Formula II where α³ is

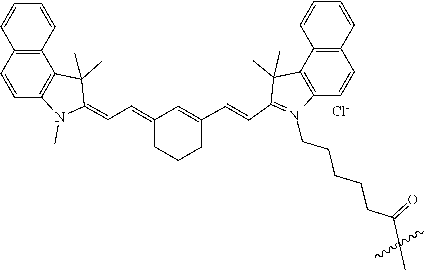 [SMILES: *C(=O)CCCCC[N+]1=C(/C=C/C2=CC(=C/C=C3/N(C)c4ccc5ccccc5c4C3(C)C)/CCC2)C(C)(C)c2c1ccc1ccccc21 ; [Cl-]]

and α⁴, α⁵, α⁶, α⁷, and α⁸ are each independently H, or a conservative amino acid substitution variant thereof, a pharmaceutically acceptable salt thereof, and/or a solvate thereof.

In another aspect, the present technology provides a protein of SEQ ID NO: 1, or a conservative amino acid substitution variant thereof, a pharmaceutically acceptable salt thereof, and/or a solvate thereof. The protein may be an isolated protein. The proteins of the present technology are useful in treating pain in a subject while avoiding the deleterious side effects typically elicited by analgesics.

In an aspect, a composition is provided that includes a compound of any aspect or embodiment disclosed herein and a pharmaceutically acceptable carrier or one or more excipients, fillers or agents (collectively referred to hereafter as “pharmaceutically acceptable carrier” unless otherwise indicated and/or specified). In a related aspect, a medicament is provided that includes a compound of any aspect or embodiment disclosed herein. In a related aspect, a pharmaceutical composition is provided that includes (i) an effective amount of a compound of any aspect or embodiment disclosed herein, and (ii) a pharmaceutically acceptable carrier. For ease of reference, the compositions, medicaments, and pharmaceutical compositions of the present technology may collectively be referred to herein as “compositions.” In further related aspects, the present technology provides methods including a compound of any embodiment disclosed herein and/or a composition of any embodiment disclosed herein as well as uses of a compound of any embodiment disclosed herein and/or a composition of any embodiment disclosed herein. Such methods and uses may include an effective amount of a compound of any embodiment disclosed herein.

In any aspect or embodiment disclosed herein, the effective amount may be determined in relation to a subject. “Effective amount” refers to the amount of a compound or composition required to produce a desired effect. One non-limiting example of an effective amount includes amounts or dosages that yield acceptable toxicity and bioavailability levels for therapeutic (pharmaceutical) use including, but not limited to, the treatment of pain or for the imaging of peripheral neurons. In any aspect or embodiment disclosed herein (collectively referred to herein as “any embodiment herein,” “any embodiment disclosed herein,” or the like) of the compositions, pharmaceutical compositions, and methods including compounds of the present technology, the effective amount may be an imaging-effective amount of the compound for imaging peripheral neurons in a subject. An “imaging-effective amount” refers to the amount of a compound or composition required to produce a desired imaging effect, such as a quantity of a compound of the present technology necessary to be detected by the detection method chosen. For example, an effective amount of a compound of the present technology includes an amount sufficient to enable detection of binding of the compound to peripheral neurons. Another example of an effective amount includes amounts or dosages that are capable of providing a fluorescence emission (above background) in peripheral neurons in a subject, such as, for example, statistically significant emission above background. As used herein, a “subject” or “patient” is a mammal, such as a cat, dog, rodent or primate. Typically the subject is a human. In any embodiment herein of the compositions, pharmaceutical compositions, and methods including proteins of the present technology, the effective amount may be an amount that treats pain in a subject. Such treatment may include a statistically significant reduction of perceived pain over such performance absent administration of the compound; such an increase may be a statistically significant increase over such performance as compared to administration of an equivalent amount of a comparative standard in terms of moles. By way of example, the effective amount of any embodiment herein including proteins of the present technology may be from about 0.01 μg to about 200 mg of the compound per gram of the composition, and preferably from about 0.1 μg to about 10 mg of the compound per gram of the composition.

The pharmaceutical composition of any embodiment disclosed herein may be packaged in unit dosage form. The unit dosage form is effective in treating pain (when proteins of the present technology are included) or is effective in imaging peripheral neurons (when compounds of the present technology are included). Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from 0.01 mg/kg to 100 mg/kg or, preferably, from 0.1 mg/kg to 10 mg/kg. Suitable unit dosage forms, include, but are not limited to parenteral solutions, oral solutions, powders, tablets, pills, gelcaps, capsules, lozenges, suppositories, patches, nasal sprays, injectables, implantable sustained-release formulations, mucoadherent films, topical varnishes, lipid complexes, liquids, etc.

The compositions of the present technology may be prepared by mixing one or more compounds of any embodiment disclosed herein of the present technology with one or more pharmaceutically acceptable carriers in order to provide a pharmaceutical composition useful to prevent and/or treat pain (when proteins of the present technology are included) or useful in imaging peripheral neurons (when compounds of the present technology are included). Such compositions may be in the form of, for example, granules, powders, tablets, capsules, syrup, suppositories, injections, emulsions, elixirs, suspensions or solutions. The instant compositions may be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular injections. The following dosage forms are given by way of example and should not be construed as limiting the present technology.

For oral, buccal, and sublingual administration, powders, suspensions, granules, tablets, pills, capsules, gel caps, and caplets are acceptable as solid dosage forms. These can be prepared, for example, by mixing one or more compounds of the instant present technology with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, enteric coatings, controlled release coatings, binders, thickeners, buffers, sweeteners, flavoring agents, perfuming agents, or a combination of any two or more thereof. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical compositions may be prepared as liquid suspensions or solutions using a sterile liquid, such as, but not limited to, an oil, water, an alcohol, and combinations of these. Pharmaceutically suitable surfactants, stabilizers, antioxidants, suspending agents, emulsifying agents, buffers, pH modifiers, or a combination of any two or more thereof, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as, but not limited to, poly(ethylene glycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Additionally or alternatively, sterile oils may be employed as solvents or suspending agents. Typically, the oil or fatty acid is non-volatile, including natural or synthetic oils, fatty acids, mono-, di- or tri-glycerides.

For injection, the composition may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, buffers, surfactants, bioavailability modifiers, and combinations of any two or more of these.

Compounds of the present technology may be administered to the lungs by inhalation through the nose or mouth. Suitable compositions for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and non-aqueous (e.g., in a fluorocarbon propellant) aerosols may be used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically acceptable carrier and/or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remington's Pharmaceutical Sciences” Mack Pub. Co., New Jersey (1991), and “Handbook of Pharmaceutical Excipients” by Raymond Rowe. Pharmaceutical Press, London, UK (2009), each of which is incorporated herein by reference.

The compositions (e.g., pharmaceutical compositions) of the present technology may be designed to be short-acting, fast-releasing, long-acting, and sustained-releasing as described below. Thus, the compositions may also be formulated for controlled release or for slow release.

The compositions of the present technology may also include, for example, micelles or liposomes, or some other encapsulated form, or may be administered in an extended release form to provide a prolonged storage and/or delivery effect. Therefore, the compositions may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Various assays and model systems, for example those described herein, can be readily employed to determine the therapeutic effectiveness of the treatment according to the present technology.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

In any aspect or embodiment herein of methods of the present technology, administration may include but not be limited to, parenteral, intravenous, intramuscular, intradermal, intraperitoneal, intratracheal, subcutaneous, oral, intranasal/respiratory (e.g., inhalation), transdermal (topical), sublingual, ocular, vaginal, rectal, or transmucosal administration.

### EXAMPLES

The examples herein are provided to illustrate advantages of the present technology and to further assist a person of ordinary skill in the art with preparing and/or using the compounds of the present technology. The examples herein are also presented in order to more fully illustrate the preferred aspects of the present technology. The examples should in no way be construed as limiting the scope of the present technology. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Experimental Materials and Methods

General. Unless otherwise stated, all solvents and reagents were obtained from Sigma-Aldrich or Fisher Scientific and were used without further purification. BODIPY-FL was purchased from Invitrogen (Carlsbad, Calif.). Cyanine7.5 (Cy7.5) was purchased from Lumiprobe (Maryland, USA). Anti-Naᵥ1.7 antibody [N68/6] was purchased from Abcam (ab85015). Water (>18.2 MΩ cm⁻¹ at 25° C.) was obtained from an Alpha-Q Ultrapure water system (Millipore). Acetonitrile (AcN) and ethanol (EtOH) were of high-performance liquid chromatography (HPLC) grade. PBS without Ca²⁺ or Mg²⁺ was obtained from the Media Preparation Facility at Memorial Sloan Kettering Cancer Center and used for all in vivo injections. Reverse-phase (RP) HPLC purifications were performed on a Shimadzu HPLC system equipped with a DGU-20A degasser, SPD-M20A UV detector, LC-20AB pump system, and a CBM-20A communication BUS module using RP-HPLC columns (Atlantis T3 C18, 5 μm, 4.6×250 mm, P/N: 186003748). Epifluorescence imaging was performed on an IVIS Spectrum (PerkinElmer). Fluorescence stereoscope images were obtained with a Lumar fluorescence stereoscope (SteREO Luma. V12, Zeiss, Jena, Germany). Confocal microscopy images were captured using a Zeiss-LSM880 (Oberkochen, Germany) point-scanning confocal microscope equipped with a 405 nm laser for detection of Hoechst 33342, and a 488 nm laser for detection of Hsp1a-FL.

Isolation of Hsp1a from spider venom. All venoms were collected by electrical stimulation or aggravation as previously described (Klint et al., Br. J. Pharmacol. 172:2445-2458 (2015)). 2 mg of Homoeomma spec. Peru spider venom was diluted with H₂O and then loaded onto an analytical C18 reverse-phase (RP) HPLC column (Jupiter® 5 μm C18 300 Å, LC Column 250×4.6 mm; phenomenex) attached to a Prominence HPLC system (Shimadzu, Rydalmere, NSW, Australia). Components were eluted at 1 mL·min⁻¹ with solvent A [99.5% H₂O, 0.05% trifluoroacetic acid (TFA)] and solvent B [90% Methyl cyanide (MeCN), 0.05% TFA in H₂O] using isocratic elution at 5% solvent B for 5 min, followed by a gradient of 5-20% solvent B over 5 min, then 20-40% solvent B over 40 min and then 40-80% solvent B over 5 min. The fractions that inhibit hNaᵥ1.1 were further fractionated using a VisionHT HILIC HPLC column (Grace™, 150×4.6 mm, fisher scientific) and eluted at 1 mL·min-1 with the same solvent A and solvent B using isocratic elution at 95% solvent B for 3 min, followed by a gradient of 95-75% solvent B over 20 min and then 75-5% solvent B over 2 min. Absorbance was measured at 214 and 280 nm using a Shimadzu Prominence SPD-20A detector.

Sequencing of Hsp1a. Peptide mass was determined using matrix-assisted laser desorption/ionization time-of-flight (MALDI-TOF) MS using a Model 4700 Proteomics Analyser (Applied Biosystems, Foster City, Calif., USA). The HPLC fraction containing Hsp1a was spotted with α-cyano-4-hydroxycinnamic acid (7.5 mg·mL-1 in 50% MeCN). Peptide was reduced and alkylated before being sequenced. Australian Proteome Analysis Facility did the reducing, alkylation and N-terminal sequencing of Hsp1a.

Synthesis of sHsp1a. sHsp1a was synthesized using standard FMOC chemistry on a Symphony peptide synthesizer (Gyros Protein Technologies, Tucson, Ariz., USA), as previously described (Agwa et al., Biochem. Biophys. Acta 1859:835-844 (2017)). C-terminal amidation was achieved using a Rink-amide resin at a scale of 0.125 mmol. Simultaneous release from the resin and removal of side chain protecting groups occurred in a solution containing TFA/triisopropylsilane (TIPS)/water (48:1:1) (v/v/v) for 2.5 h. Crude sHsp1a was triturated in chilled diethyl ether, and then the precipitated peptide precipitate was dissolved in solvent AB (45% (v/v) acetonitrile, 0.05% TFA (v/v)), lyophilized and prepurified using C18 RP-HPLC. The peptide was eluted using a linear gradient of 10-60% solvent B (90% v/v ACN; 0.05% v/v TFA) over 50 min using a flow rate of 50 mL·min⁻¹. Fractions were collected and analyzed using electrospray ionization-mass spectroscopy (ESI-MS) and fractions of interest were pooled, lyophilized and stored at −20° C. sHsp1a (0.1 mg/mL) was oxidized for 16 h at room temperature in a buffer containing 2 M Urea, 0.1 M Tris pH 8, 0.15 mM reduced glutathione and 0.3 mM oxidized glutathione. Oxidation was quenched by acidification to pH 3, before the peptide was filtered and purified using preparatory and semi-preparatory RP-HPLC as previously described (Agwa et al., J. Biol. Chem. 293(23):9041-9052 (2018)). The mass of synthetic Hsp1a was verified via matrix-assisted laser desorption/ionization time-of-flight (MALDI-TOF) MS by a Model 4700 Proteomics Analyser (Applied Biosystems, Foster City, Calif., USA). LCESI-MS (ES+), m/z calculated for [C₁₄₈H₂₂₀N₄₀O₄₀S₆] 3389.52, [C₁₄₈H₂₂₀N₄₀O₄₀S₆+2H]²⁺ 1696.76, found [M+2H]²⁺ 1696.00, [C₁₄₈H₂₂₀N₄₀O₄₀S₆+3H]³⁺ 1130.84, found [M+3H]³⁺ 1131.05, [C₁₄₈H₂₂₀N₄₀O₄₀S₆+4H]⁴⁺ 848.38, found [M+4H]4+ 848.55, [C₁₄₈H₂₂₀N₄₀O₄₀S₆+5H]⁵⁺ 678.90, found [M+5H]⁵⁺ 679.05.

Synthesis of Hsp1a-FL. Hsp1a peptide (0.37 mM, 250 μg in 200 μL of ACN) and Na2CO3 (1 M, 40 μL) were transferred into a 3 mL amber vial with a magnetic bar stirrer. BODIPY-NHS (4 μL of a 26 mM solution) was dissolved in ACN and added dropwise to the reaction mixture. The final volume of the reaction mixture was 350 μL. The reaction mixture was allowed to stir for 10-20 min before dilution with 100 μL of water. This reaction afforded the mono- and diadduct, which were purified and separated using RP-HPLC. Fractions containing Hsp1a-FL were concentrated and solvent removed in vacuo to afford a red-yellowish powder (150 μg, 55% yield from Hsp1a peptide). This purified compound was formulated in 100% Ca²⁺/Mg²⁺-free PBS or 10% dimethyl sulfoxide (DMSO) and PBS. LC-ESI-MS (ES+), m/z calculated for [C₁₆₂H₂₂₈BF₂N₄₃O₄₀S₆] 3663.63, [C₁₆₂H₂₂₈BF₂N₄₃O₄₀S₆+2H]²⁺ 1832.83, found [M+2H]²⁺ 1833.10, [C₁₆₂H₂₂₈BF₂N₄₃O₄₀S₆+3H]³⁺ 1222.22, found [M+3H]³⁺ 1222.40, [C₁₆₂H₂₂₈BF₂N₄₃O₄₀S₆+4H]⁴⁺ 916.91, found [M+4H]⁴⁺ 917.05.

Hsp1a-IR800, Hsp1a-DY-684, Hsp1a-Janelia669, Hsp1a-BODIPY665, and Hsp1a-Cy7.5 were synthesized via essentially a similar procedure as noted above for Hsp1a-FL.

Synthesis of Hs1a. Hs1a was synthesized using expression in the periplasm of E. coli. The recombinant peptide containing a non-native N-terminal glycine residue was purified by nickel affinity chromatography after liberation from the fusion protein with TEV protease cleavage. LC-ESI-MS (ES+), m/z calculated for [C₁₆₄H₂₅₁N₄₉O₄₇S₆] 3850.74, [C₁₆₄H₂₅₁N₄₉O₄₇S₆+3H]³⁺ 1284.58, found [M+3H]³⁺ 1285.00, [C₁₆₄H₂₅₁N₄₉O₄₇S₆+4H]⁴⁺ 963.69, found [M+4H]⁴⁺ 964.20, [C₁₆₄H₂₅₁N₄₉O₄₇S₆+5H]⁵⁺ 771.15, found [M+5H]⁵⁺ 772.60, [C₁₆₄H₂₅₁N₄₉O₄₇S₆+6H]⁶⁺ 642.79, found 643.25.

Synthesis of Hs1a-FL. Hs1a peptide (0.26 mM, 200 μg in 200 μL of ACN) and Na2CO3 (1M, 40 μL) were transferred into a 3 mL amber vial with a magnetic bar stirrer. Cy7.5-NHS (4 μL of a 24 mM solution) was dissolved in ACN and added dropwise to the reaction mixture. The final volume of the reaction mixture was 350 μL. The reaction mixture was allowed to stir for 10 min before dilution with 100 μL of water. This reaction afforded the mono- and di-adduct, which were purified and separated using RP-HPLC. Fractions containing Hs1a-FL were concentrated and solvent removed in vacuo to afford a dark greenish powder (20 μg, 14% yield from Hs1a peptide). This purified compound was then formulated in 100% Ca2+/Mg2+-free PBS or 10% dimethyl sulfoxide (DMSO) and PBS. LC-ESI-MS (ES+), m/z calculated for [C₂₀₉H₂₉₈N₅₁O₄₈S₆] 4482.12, [C₂₀₉H₂₉₈N₅₁O₄₈S₆+3H]³⁺ 1495.04, found [M+3H]³⁺ 1495.45, [C₂₀₉H₂₉₈N₅₁O₄₈S₆+4H]⁴⁺ 1121.53, found [M+4H]⁴⁺ 1121.75, [C₂₀₉H₂₉₈N₅₁O₄₈S₆+5H]⁵⁺ 897.42, found [M+5H]⁵⁺ 897.75, [C₂₀₉H₂₉₈N₅₁O₄₈S₆+6H]⁶⁺ 748.02, found [M+6H]⁶⁺ 748.25.

Tryptic Digest of Hsp1a-FL and Hsp1a. 15 μL of digestion buffer and 1.5 μL of reducing buffer were added to a 0.5 mL microcentrifuge tube. 10.5 μL of a solution containing 4 μg of Hsp1a-FL was added to the same tube, and then the final volume was adjusted to 27 μL with ultrapure water. The sample was then incubated at 95° C. for 5 min. The sample was allowed to cool to room temperature before 2 μL of activated trypsin were added, followed by incubation at 37° C. overnight. The sample was analyzed with LC-ESI-MS. LC-ESI-MS (ES+), m/z calculated for the fragmentation of Hsp1a-FL was observed as follows, for fragment [C₁₂₆H₁₈₅N₃₃O₃₄S₅] 2864.24, m/z calculated for [C₁₂₆H₁₈₅N₃₃O₃₄S₅+2H]²⁺ 1433.12, found [M+2H]²⁺ 1432.95, [C₁₂₆H₁₈₅N₃₃O₃₄S₅+3H]³⁺ 955.75, found [M+3H]³⁺ 956.00.

Hsp1a was digested following the same tryptic digestion protocol and the sample was analyzed with LC-ESI-MS. LCESI-MS (ES−), m/z calculated for the fragmentation of Hsp1a was observed as follows, one fragment [C₁₁₂H₁₇₁N₃₁O₃₃S₅] 1318.70 and second fragment [C₉₀H₁₃₈N₂₄O₂₇S₄] 1056.45, m/z calculated for [C₁₁₂H₁₇₁N₃₁O₃₃S₅-2H]²⁻ 1318.07, found [M−2H]²⁻ 1318.70, m/z calculated for [C₉₀H₁₃₈N₂₄O₂₇S₄] 1057.45, found [M−2H]²⁻ 1057.10. In addition, for Hsp1a, the following fragmentation was observed in LCESI-MS (ES+), m/z calculated for [C₉₀H₁₃₈N₂₄O₂₇S₄+2H]²⁺ 1058.45, found [M+2H]²⁺ 1058.80, [C₉₀H₁₃₈N₂₄O₂₇S₄+3H]³⁺ 705.97, found [M+3H]3+ 706.35.

Cell Lines. HEK293 cells stably expressing the human NaV channel (31 subunit (hNaVβ1) in combination with the a subunit of hNaᵥ1.1, hNaᵥ1.2, hNaᵥ1.3, hNaᵥ1.4, hNaᵥ1.5, hNaᵥ1.6 or hNaᵥ1.7 (Scottish Biomedical, Glasgow, UK) were cultured in DMEM/F-12 media (1:1), supplemented with 10% fetal bovine serum, 400 mg/mL geneticin and 100 mM non-essential amino acids (all reagents from Invitrogen) at 37° C. and in 5% CO₂.

Recombinant expression and purification of rHsp1a. The full-length DNA fragment encoding Hsp1a was synthesised by GeneArt (Thermofisher) and cloned into the pLiCc vector that encodes a His6-Maltose binding protein (MBP) tag (“His6” disclosed as SEQ ID NO: 12) at the N-terminus of the toxin. Recombinant Hsp1a (rHsp1a) was produced using minor modifications of a previously described method. Toxin overexpression was induced overnight at 25° C. by 0.5 mM IPTG when the cell density (OD_{600 nm}) reached 0.6-0.8. Following Ni-NTA purification of the fusion protein, Hsp1a was cleaved from the His6-MBP fusion tag (“His6” disclosed as SEQ ID NO: 12) with TEV protease and purified via reverse-phase HPLC using a semi-preparative Ascentis® C4 column. The column was pre-equilibrated with 90% solvent A and 10% solvent B, then peptide was eluted at a flow rate of 3 ml/min using a gradient of 10-60% solvent B over 30 min. Hsp1a was further purified using an analytical Ascentis® C18 RP-HPLC column pre-equilibrated with 15% Solvent A and 85% Solvent B. Peptide was eluted at a flow rate of 0.8 mL/min using a gradient of 15-45% Solvent B over 40 min. The mass of recombinant Hsp1a was verified via MALDI-TOF.

Co-elute sHsp1a, rHsp1a with Hsp1a. 1.5 μL of native Hsp1a, synthetic Hsp1a (sHsp1a), recombinant Hsp1a (rHsp1a) and the mixture of Hsp1a and sHsp1a was mixed with 20 μL of 10% solvent B+90% solvent A. 15 μL of each sample was injected into an Aeris™ 3.6 μm PEPTIDE XB-C18 column (50×2.1 mm; Phenomenex). RP-HPLC was performed on a Shimadzu LC20AT system and peptides were eluted at a flow rate of 0.3 mL/min using a gradient of 10-50% solvent B over 30 min.

Electrophysiology. Functional characterization of voltage-gated sodium currents was accomplished using either conventional whole cell patch clamp or Qpatch 16X high-throughput electrophysiology platforms (Sophion Bioscience, Denmark).

Qpatch 16X high-throughput electrophysiology platforms (Sophion). Whole-cell patch-clamp experiments were performed at room temperature using a QPatch 16× automated electrophysiology platform (Sophion Bioscience, Denmark) using 16-channel planar patch-chip plates (QPlates) with a patch-hole diameter of 1 μm and resistance of 2 MΩ Whole-cell currents were filtered at 5 kHz (8-pole Bessel) and digitized at 25 kHz. A P4 online leak-subtraction protocol was used with non-leak-subtracted currents acquired in parallel.

HEK293 cells stable expressing the hNaᵥ1.1-1.7α and the human β1 subunits (SB drug discovery) were used to examine the effect of peptides on Naᵥ channels. HEK293-hNaᵥ cells were seeded into a 175 cm² cell culture flask two days prior to patching and were detached at 70% confluency using 2 mL Detachin™ (Genlantin, San Diego, Calif., USA). The cells were pelleted at 8000 rpm for 8 min. After removing the supernatant, cells were resuspended in 5 mL QPatch media containing 96.5% CD293 medium, 25 mM HEPES (Gibco) and 1× glutamine (Gibco). The extracellular solution was 2 mM CaCl₂, 1 mM MgCl₂, 10 mM HEPES, 4 mM KCl, 145 mM NaCl, 10 mM Glucose, pH 7.4 and the intracellular solutions was 140 mM CsF, 1 mM/5 mM EGTA/CsOH, 10 mM HEPES, 10 mM NaCl, pH 7.3.

All peptides were dissolved in the extracellular solution with 0.1% bovine serum albumin (BSA). Concentration—response data were obtained using five concentrations of peptide (0.2, 2, 20, 200, and 2000 nM) for Hsp1a-FL. HEK293-hNaV cells were clamped at a holding potential of −80 mV, then 10 μL of peptide was applied immediately for 6 s before applying the voltage protocol: −80 mV for 10 ms, −120 mV for 200 ms, 0 mV for 20 ms, then return to holding potential of −80 mV. The total incubation time for each peptide concentration was 7.5 min: the voltage protocol was applied 15 times, with cells clamped at a holding potential of −80 mV for 30 s between runs. Concentration response data were analysed using nonlinear least-squares fit of the log (inhibitor) vs. response (three parameters) via GraphPad Prism 8 to provide pIC50 determinations.

For HEK293-hNaᵥ cells (hNaᵥ1.1, hNaᵥ1.2, hNaᵥ1.3, hNaᵥ1.4, hNaᵥ1.5, hNaᵥ1.6 or hNaᵥ1.7), concentration-response data were obtained using five concentrations of peptide (2 nM-10 μM). HEK293-hNaᵥ cells were clamped at a holding potential of (−60 mV for Naᵥ1.1, −65 mV for Naᵥ1.2, −60 mV for Naᵥ1.3, −75 mV for Naᵥ1.4, −105 mV for Naᵥ1.5, −60 mV for Naᵥ1.6 and −75 mV for Naᵥ1.7). For each concentration, 10 μL of peptide were added for 6 seconds before applying the following voltage: −80 mV for 10 ms, −120 mV for 200 ms, 0 mV for 20 ms, then return to the holding potential of −80 mV. This was repeated once every 60 s during liquid applications. Cells were otherwise held at the holding potential when the above voltage protocol was not executed. Upon establishment of the whole-cell recording configuration, a total of five applications of the extracellular solution (1× control buffer, 3× test compound/control, 1 μM TTX (positive control), all containing 0.1% BSA, except for the TTX solution) were made on each cell. The voltage protocol was executed 10 times after each application. Currents were sampled at 25 kHz and filtered at 5 kHz with an 8-pole Bessle filter. The series resistance compensation level was set at 80%. All experiments were performed at room temperature (˜22° C.). IC₅₀ values were determined from logistic fits of concentration-response data using GraphPad Prism 7.

Conventional patch clamp. Coverslips containing cells of HEK293 stable expressing hNaᵥ1.7 α and human β1 subunits were placed in a recording chamber on the stage of an inverted microscope with the extracellular solution containing 140 mM NaCl, 2 mM CaCl₂, 4 mM KCl, 1 mM MgCl₂, and 10 mM HEPES (pH 7.4 with NaOH). Recording patch pipettes were filled with an intracellular solution containing 120 mM CsCl, 30 mM NaCl, 1 mM/5 mM EGTA/CsOH and 10 mM HEPES (pH 7.2 with CsOH) and had a resistance of 1-3 MΩ. All recordings were made at room temperature (22-24° C.) using MultiClamp 700B amplifier, Axo™ Digidata® 1550B and PCLAMP software (Molecular Devices). Naᵥ channel currents were measured using the whole cell configuration of the patch clamp technique. All peptides were dissolved in the extracellular solution with 0.1% BSA. The effects of peptides were tested by using the same voltage protocol as showing in the Qpatch technique. Concentration response data were analysed using nonlinear least-squares fit of the log (inhibitor) vs. response (three parameters) (GraphPad Prism 7) to provide pIC50 determinations. The conductance-voltage (G-V) relationships obtained by using a protocol in which cells depolarised from a holding potential of −120 mV, stepping from −80 to +60 mV in 5 mV increments. Steady-state inactivation was obtained by applying a depolarisation to 0 mV following a series step potential from −120 mV to 20 mV in 5 mV increments. The V_{0.5} activation and inactivation were calculated by fitting data with Boltzmann equation in GraphPad Prism 7. Recovery from fast inactivation was obtained by applying a first 0 mV pulse followed by repolarisation to −120 mV and a second pulse to 0 mV after a period ranging from 0 to 50 ms, increasing in 1 ms increments. The time constant was calculated by fitting data with one-phase decay in GraphPad Prism 7.

NMR spectroscopy. A 600 MHz Bruker Avance NMR spectrometer (Bruker Biospin Callerica, Mass.) equipped with a cryoprobe was used to obtain NMR spectra using 1 mg/mL peptide dissolved in 10% D2O, 90% H₂O (v/v) pH˜4 at 298 K. One-dimensional (1D) ¹H and two-dimensional ¹H-¹H TOCSY (80 ms mixing time) and NOESY (200 ms mixing time) spectra were collected, processed using TopSpin version 3.5 (Bruker) and used in the sequential assignment of the amino acid residues which was done on CCPNMR Analysis 2.4.1 (CCPN, University of Cambridge, Cambridge, UK) (Vranken et al., Proteins 59:687-696 (2005); Wüthrich K, NMR OF PROTEINS AND NUCLEIC ACIDS (New York, Wiley Interscience 1st ed. 1986). Additional experiments for the structure calculation included amide proton temperature coefficient experiments (283-308 K in 5 K increments) for amide proton temperature coefficients and ¹H-¹⁵N HSQC in 10% D₂O, 90% H₂O (v/v). Experiments in 100% (v/v) D₂O, included ¹D ¹H spectra, ¹H-¹³C HSQC and ECOSY experiments.

Several rounds of the AUTO and ANNEAL functions on CYANA 3.97 (Güntert group, Goethe University Frankfurt, Frankfurt, Germany) (Guntert, Methods Mol. Biol. 278:353-378 (2004)) were used to refine peak assignments and the final list of inter-proton distances was generated. TALOS-N (Bax Group, NIH, MD, USA) was used to determine and ψ dihedral angles using Hα, Cα, Cβ and HN chemical shifts from NOESY, ¹H-¹³C, and ¹H-¹⁵N spectra (Shen & Bax, 2013). H-bond restraints were obtained from D₂O exchange experiments, the temperature coefficient data and direct measurements on preliminary structures. χ1 angles were derived from the E.COSY spectrum in combination with NOE intensities. The structure was further refined in a water shell using protocols in the RECOORD database (Brünger et al., Acta Crystallogr. D. Biol. Crystallogr. 54(Pt 5):905-21 (1998); Nederveen et al., Proteins 59:662-672 (2005)), and the final set of 20 structures was determined from lowest energy, best MolProbity scores and fewest distance and dihedral angle violations.

Animal Model. Female athymic nude mice (4-8 week-old, athymic-Nude (outbred) (Stock #: 088; Envigo, USA) were allowed to acclimatize at the Memorial Sloan Kettering Cancer Center (MSK) vivarium for 1 week with ad libitum food and water prior to the experimental procedure. For imaging experiments, animals were sacrificed 30 min post-tail vein injection of Hs1a-FL, Hs1a/Hs1a-FL, Hsp1a-FL, Hsp1a/Hsp1a-FL, or PBS. All animal experiments were performed in accordance with institutional guidelines and approved by the IACUC of MSK, following NIH guidelines for animal welfare.

Mouse 3D sliced model. A mouse was fast frozen after euthanasia in liquid nitrogen. The animal was sliced and imaged by EMIT using a Xerra imager. Each sectioned slice was 50 microns. A 3D model of the sliced mouse was reconstructed using 3D slicer software.

Human Tissue. Human nerves (vagus nerves, n=3) were a donation from the Fusion Solutions Bioskills Laboratory, Long Island, N.Y. The nerves were paraffin-embedded and formalin fixed and sectioned at 10 μm thickness for H&E and immunohistochemical detection experiments.

Immunohistochemistry. Naᵥ1.7 in human vagus nerves and mouse sciatic nerves was detected using immunohistochemical (IHC) staining techniques, which were performed at the Molecular Cytology Core Facility of MSK using the Discovery XT processor (Ventana Medical System, Tucson, Ariz.). Anti-Naᵥ1.7 antibody [N68/6] (Abcam ab85015) specifically bound to both human and mouse Naᵥ1.7 (0.5 μg/mL). Paraffin-embedded formalin-fixed 10 μm sections were deparaffinized with EZPrep buffer. For IHC detection, a 3,3′-diaminobenzidine (DAB) detection kit (Ventana Medical Systems, Tucson, Ariz.) was used according to the manufacturer's instructions. Adjacent sections were stained against IgG, to control for non-specific binding of the sodium channel Naᵥ1.7. Sections were counterstained with H&E and coverslipped with Permount (Fisher Scientific, Pittsburgh, Pa.).

Confocal Microscopy. 5 or 10 μm cryosections of OCT-embedded sciatic nerve tissues from mice intravenously injected with Hsp1a-FL (7 nmol, 68 μM of Hsp1a-FL in 100 μL of PBS), Hsp1a/Hsp1a-FL (Hsp1a-FL, 68 μM, 7 nmol, and Hsp1a 204 μM, 21 nmol in 100 μL PBS), Hs1a-FL (4 nmol, 45 μM of Hs1a-FL in 100 μL of PBS), with the block Hs1a/Hs1a-FL (Hs1a-FL, 45 μM, 4 nmol and Hs1a 120 μM, 12 nmol in 100 μL PBS) or PBS were used to determine the distribution and localization of fluorescently-tagged peptide. Animal organs were incubated with Hoechst 33342 (blue, 20 μM, 1 nmol in 50 μL of PBS) to counterstain nuclei before embedding in Mowiol mounting medium. Fresh tissue samples were counterstained with 33342 (blue, 20 μM, 1 nmol in 50 μL of PBS) up to 90 min post-mortem and placed directly on a microscope slide for imaging.

Epifluorescence Imaging. Animals were intravenously injected with Hsp1a-FL (7 nmol, 68 μM of Hsp1a-FL in 100 μL of PBS, n=9), or Hs1a-FL (4 nmol, 45 μM of Hs1a-FL in 100 μL of PBS, n=3). To assess the specificity of the Hsp1a-FL or Hs1a-FL accumulation, animals were injected with a combination of Hsp1a and Hsp1a-FL (Hsp1a-FL, 68 μM, 7 nmol and Hsp1a 204 μM, 21 nmol in 100 μL PBS, n=9), Hs1a and Hs1a-FL (Hs1a-FL, 45 μM, 4 nmol and Hs1a, 120 μM, 12 nmol in 100 μL PBS, n=3) or PBS (n=9). Animals were sacrificed 30 min post-injection and epifluorescence images obtained. Epifluorescence images of the right sciatic nerve (RSN) and the left sciatic nerve (LSN) were obtained in situ. Epifluorescence of the biodistribution of the peptides in excited RSN, LSN, muscle, heart, kidney, liver, and brain were acquired with IVIS Spectrum (PerkinElmer) using a predefined GFP filterset (e.g., excitation=465/30 nm, emission=520-580 nm) based on the particular fluorophore employed in the compounds. Autofluorescence was removed through spectral unmixing. Semiquantitative analysis of the Hsp1a-FL or Hs1a-FL signal was conducted by measuring the average radiant efficiency (in units of unit [p/s/cm²/sr]/[μW/cm²]) in regions of interest (ROIs) that were placed on all resected organs under white light guidance.

Fluorescence Stereoscope Imaging. The Hsp1a-FL fluorescence signal in mouse sciatic nerves was also visualized using a fluorescence stereoscope 30 min after intravenous injection of Hsp1a-FL (7 nmol, 68 μM of Hsp1a-FL in 100 μL of PBS) or PBS (n=3/group). Fluorescence images were obtained using mice with exposed but otherwise intact sciatic nerves. Images were also obtained from excised sciatic nerves and muscle. Imaging was performed in bright field and fluorescence mode, with a 500/20 nm laser excitation and 535/30 emission filter and an exposure time of 200-400 ms.

Stability test of sHsp1a in human plasma. The stability of sHsp1a in human plasma was tested by adding 10 μM sHsp1a to a pooled human serum (purchase from Sigma Aldrich) and incubating at 37° C. for periods up to 24 hours. Triplicate samples were collected at each time point. The reaction mixture was precipitated at the desired time by the addition of 5 μl of 5% TFA and 10 μl of 5% formic acid. 5 μL sample at each time point was processed by LC/MS using a phenomenex C18 column (150 mm×2.1 mm, particle size 5 μm, 100 Å pore size) at a flow of 0.25 ml/min and a gradient of 1-50% solvent B (90% MeCN, 0.1% formic acid) in solvent A (0.1% formic acid) over 14 min coupled with an AB SCIEX 5600 Triple time-of-flight (TOF) mass spectrometer (cycle time 0.2751 s). Peptide areas were measured at quadruple-charge state, and were analysed using PeakView and MultiQuant (Applied Biosystems, Inc., Foster City, Calif.). Human Atrial Natriuretic Peptide (hANP, 10 μM) from GenScript (Piscataway, N.J., USA) was used as a positive control, and w-conotoxin MVIIA (MVIIA, 10 μM) from Alomone labs (Jerusalem, Israel) was used as a negative control.

Model of Chronic Visceral Hypersensitivity (CVH). All experiments were performed in accordance with the guidelines of the Animal Ethics Committees of the South Australian Health and Medical Research Institute (SAHMRI) and Flinders University. Male C57 BL/6 mice were used in all experiments.

Colitis was induced by administration of Trinitrobenzene Sulphonic acid (TNBS) as described previously (Castro et al., Gut 66(6):1083-1094 (2017); Osteen et al., Nature 534: 494-499 (2016)). Briefly, 13-week-old mice were fasted overnight with access to 5% glucose solution. After the fasting period, isofluorane anaesthetized mice were administered an intracolonic enema of 0.1 mL TNBS (130 m/mL in 30% EtOH) via a polyethylene catheter inserted 3 cm past the anus. Mice were then individually housed with unlimited access to soaked food and 5% glucose solution and were subsequently observed daily for changes in body weight, physical appearance and behaviour. Histological examination of mucosal architecture, cellular infiltrate, crypt abscesses, and goblet cell depletion confirmed TNBS induced significant damage by day 3 post-treatment, largely recovered by day 7 and fully recovered by day 28 post-treatment. High-threshold nociceptors from mice on day 28 post-treatment display significant mechanical hypersensitivity, lower mechanical activation thresholds (Hughes et al., Gut 58: 1333-1341 (2009)) and display hyperalgesia and allodynia (Adam et al., Pain 123:179-186 (2006)). Additionally, these mice exhibit an increased neuronal activation in the dorsal horn of the spinal cord in response to noxious colorectal distension, as well as sprouting of colonic afferent terminals within the dorsal horn has also been reported (Harrington et al., J. Comp. Neurol. 520:2241-2255 (2012)). Thus, they are therefore termed ‘Chronic Visceral Hypersensitivity’ (CVH) mice (Castro et al., Gut 66(6):1083-1094 (2017); Osteen et al., Nature 534: 494-499 (2016); and Hughes et al., Gut 58: 1333-1341 (2009)).

In vivo Visceromotor Responses (VMR) to Colorectal Distension (CRD). Noxious distension of the colorectum triggers the VMR, a nociceptive brainstem reflex consisting of the contraction of the abdominal muscles (Ness & Gebhart, Brain Res. 450:153-169 (1988)). Using abdominal electromyography (EMG), this technique allows assessment of visceral sensitivity in vivo in fully awake animals (Christianson & Gebhart, Nat. Protoc. 2:2624-2631 (2007); Deiteren et al., Gut 63:1873-1882 (2014)). Under isoflurane anaesthesia, the bare endings of two Teflon-coated stainless steel wires (Advent Research Materials Ltd, Oxford, UK) were sutured into the right abdominal muscle and tunneled subcutaneously to be exteriorized at the base of the neck for future access. At the end of the surgery, mice received prophylactic antibiotic (Baytril®; 5 mg/kg s.c.) and analgesic (buprenorphine; 0.4 mg/10 kg s.c.), were housed individually and allowed to recover for at least three days before assessment of VMR. On the day of VMR assessment, mice were briefly anaesthetized using isoflurane and received a 100 μl enema of vehicle (sterile saline) or the peptide sHsp1a (200 nM). A lubricated balloon (2 cm length) was gently introduced through the anus and inserted into the colorectum up to 0.25 cm past the anal verge. The balloon catheter was secured to the base of the tail and connected to a barostat (Isobar 3, G&J Electronics, Willowdale, Canada) for graded and pressure-controlled balloon distension. Mice were allowed to recover from anaesthesia in a restrainer with dorsal access for 15 minutes prior to initiation of the distension sequence. Distensions were applied at 20-40-5-60-70-80 mmHg (20 s duration) at a 2 min-interval so that the last distension was performed 30 min after i.c. treatment. Following the final distension mice were humanely killed, by cervical dislocation. The EMG electrodes were relayed to a data acquisition system and the signal was recorded (NL100AK headstage), amplified (NL104), filtered (NL 125/126, Neurolog, Digitimer Ltd, bandpass 50-5000 Hz) and digitized (CED 1401, Cambridge Electronic Design, Cambridge, UK) to a PC for off-line analysis using Spike2 (Cambridge Electronic Design). The analog EMG signal was rectified and integrated. To quantify the magnitude of the VMR at each distension pressure, the area under the curve (AUC) during the distension (20 s) was corrected for the baseline activity (AUC pre-distension, 20 s).

Colonic compliance. Colonic compliance was assessed by applying graded volumes (40-200 μL, 20 s duration) to the balloon in the colorectum of fully awake mice, while recording the corresponding colorectal pressure as described previously (Deiteren et al., Gut 63:1873-1882 2014).

Acute in vivo toxicity. All animal experiments were performed in accordance with protocols approved by the Institutional Animal Care and Use Committee of MSK and followed the National Institute of Health guidelines for animal welfare. Six athymic nude mice 6-8 weeks old were purchased from Envigo RMS, Inc. An intravenous (IV) catheter was placed in tail vein of each mouse and they were anesthetized using Isoflurane (Novaplus, Telangana-India). Anesthesia was maintained using 1.0 to 1.5 L/min of Isoflurane and 2 L/min of oxygen. Three animals were injected with 68 μM, 7 nmol Hsp1a-FL in 100 μL of PBS, and the other three with 1004, of PBS. Animals were monitored using a rodent surgical monitor (Indus instruments, Houston, Tex.) before, during, and up to 16 minutes after injection. Numeric data points represent averaged values of 5±1 seconds duration. Data was collected before injection, 15 seconds after injection, 5 minutes after injection and 15 minutes after injection. Mouse body core temperature was measured by placing a rectal probe and electronically regulated via a surgical platform. The high-resolution electrocardiogram (EKG) was measured by placing non-invasive electrodes on the 4 paws and electrical contact was assured using a conducting gel (Electrode cream, Indus instruments, Houston, Tex.). Heart rate was automatically calculated from the R-R peaks derived from the EKG signal. Peripheral capillary oxygen saturation (SpO2) was non-invasively measured by placing a clip sensor on the animal's left thigh.

Statistical Analysis. Data were statistically analyzed by generalized estimating equations followed by LSD post hoc test when appropriate using SPSS 23.0. During animal toxicity studies, data points representing loss of signal were excluded from the analysis. Loss of signal was defined by a brief contact loss between the monitoring equipment and the mice (creating a “false” zero or −1 result). Loss of signal was corrected by placing the electrode in close contact to the mice. Average and standard deviations were calculated for each mouse.

Unless otherwise stated, data points represent mean values and error bars represent the standard deviation of biological replicates (mean±SEM). All p-values were calculated using a Student's unpaired t-test. Statistical significance was considered for p-values<0.05 and as follows: ns=not significant, *p<0.05, **p<0.01, ***p<0.001. Mann-Whitney tests were used for analysis of the unpaired samples (e.g. vital signs in mice injected with Hsp1a and mice from the control group) and the Wilcoxon test was used for analysis of paired samples (e.g. vital signs from same mouse before and after injection). Statistical significance was determined with alpha=0.05. Analysis and figures were prepared in GraphPad Prism 8.

### Example 2: Isolation of μ-Theraphotoxin-Hsp1a (Hsp1a) from Homoeomma spec. Peru

Physiological Screen for the Identification of Bioactive Venom Peptides from Spider and Scorpion Crude Venoms.

To identify novel bioactive venom peptides that could inhibit the activity of voltage-gated sodium channels, crude venom from 52 species of spider and 12 species of scorpion were screened against hNaᵥ1.1 via an automated whole-cell patch-clamp electrophysiology system QPatch 16X. Of the 20% of the venoms that showed inhibitory activity to hNaᵥ1.1, the crude venom from Homoeomma spec. Peru was further characterized because 20 ng/μL of the venom strongly inhibited hNaV1.1.

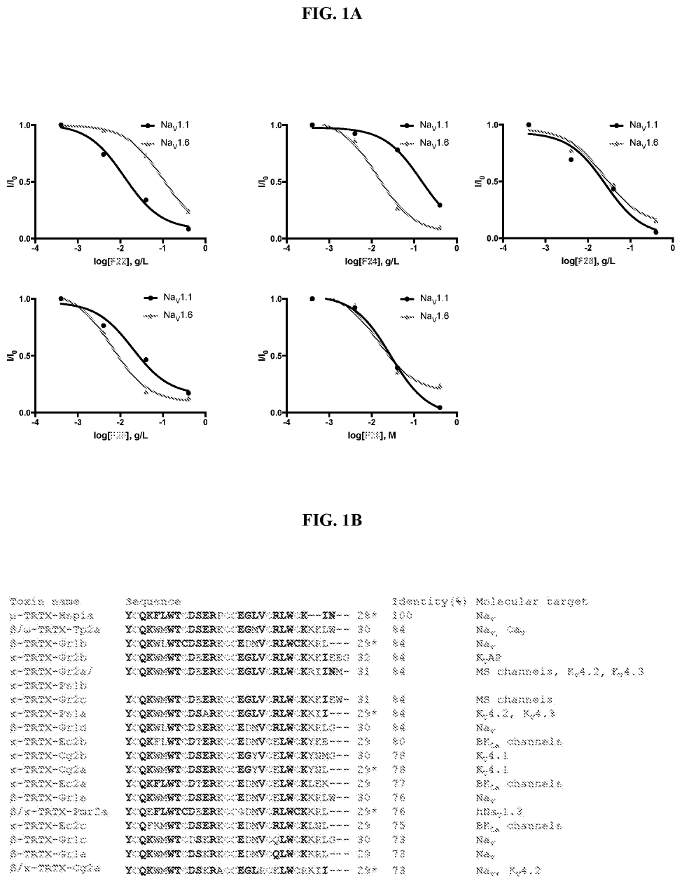

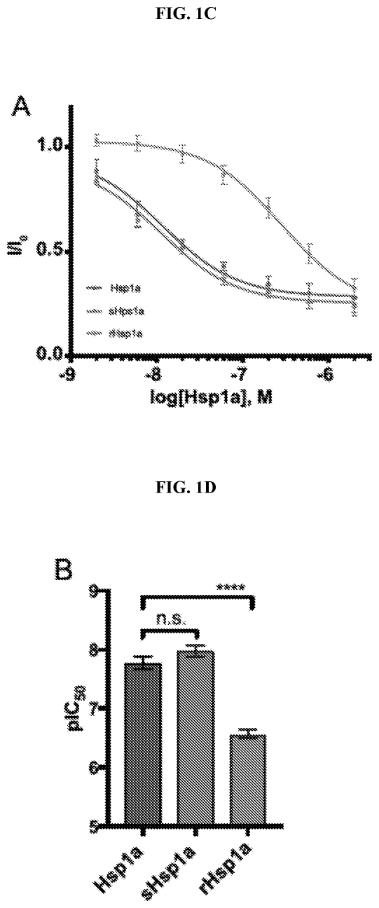

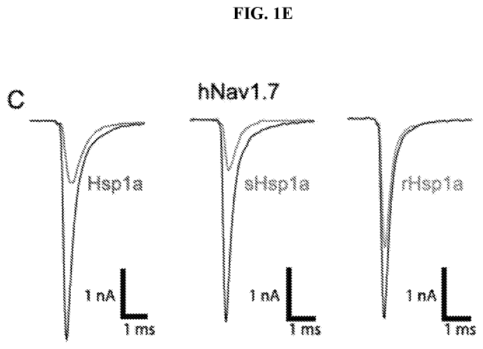

The venom was fractionated using an analytical C18 RP-HPLC column and six of the isolated fractions showed promising inhibitory activity on hNaᵥ1.1. Fraction F22, which exhibited selectivity between hNaᵥ1.1 and hNaᵥ1.6 (FIG. 1A). Fraction F22 was further purified by a HILIC RP-HPLC column to obtain a pure peptide with the observed monoisotopic mass of 3388.589 Da by MALDI-TOF MS. The peptide was named μ-theraphotoxin-Hsp1a because it is the first peptide isolated from Homoeomma spec. Peru and the μ prefix denoting inhibition of Naᵥ channels. The N-terminal sequencing revealed that the peptide contains 28 amino acid with a calculated monoisotopic oxidized mass of 3388.526 Da, consistent with the presence of six oxidized cysteine residues and an amidated C-terminus (FIG. 1B). The sequence blast of Hsp1a in ArachnoServer indicated that Hsp1a was a novel and uncharacterized venom peptide (FIG. 1B). The amino acid sequence of Hsp1a shared at least 70% sequence identity with the spider venom peptides from the NaSpTx family 3 (FIG. 1B).

### Example 3: Hsp1a Production Via Chemical Synthesis and Recombinant Expression

Production of Synthetic (sHsp1a) and Recombinant (rHsp1a) Hsp1a

To get enough material for the characterization of Hsp1a, chemical synthesis was used to produce a synthetic Hsp1a peptide (sHsp1a). A single sharp peak was obtained from the final analytical RP-HPLC purification and 96% purity was achieved as calculated from area under the curve. The observed monoisotopic mass 3389.589 Da by MALDITOF MS indicated the successful oxidation of sHsp1a with three disulfide bridges and the C-terminal amidation.

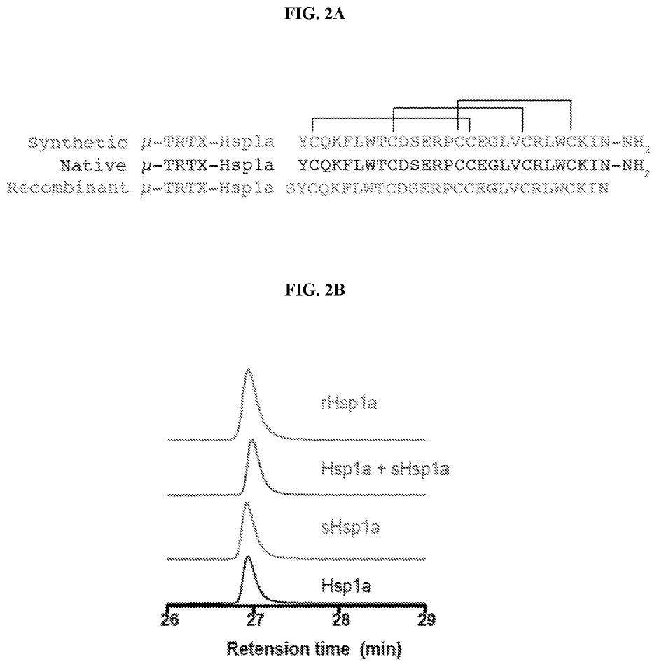

The Hsp1a was also produced using recombinant periplasmic expression in Escherichia Coli (E. coli). An artificial serine was introduced at the N-terminus of the recombinant Hsp1a (rHsp1a) to assist TEV cleavage and the C-terminus is amidated by recombinant expression (FIG. 2A). The yield of rHsp1a was ˜50 μg/L cell culture. The final analytical RP-HPLC purification of the rHsp1a showed a single peak indicating that rHsp1a forms a single isomer after releasing from the MBP fusion tag. MALDI-TOF MS revealed a monoisotopic mass of 3476.614 Da, which was consistent with the presence of an additional serine at the N-terminus and a non-amidated C-terminus. As shown in FIG. 2B, the sHsp1a and rHsp1a co-eluted at the same time as the native Hsp1a suggesting that the sHsp1a and rHsp1a folded correctly. Therefore, Hsp1a can be successfully produced in the same folding as the native material by either chemical synthesis or recombinant expression.

NMR Structure of sHsp1a

Characterization of Hsp1a structure was determined by 2D 1H NMR. Table 1 shows the energies and structural statistics for the final 20 structures of sHsp1a. This analysis showed that the six cysteine residues of sHsp1a form a characteristic inhibitor cystine knot (ICK) motif with Cys 2-16, Cys 9-21, and Cys 15-25 connectivity and in a fashion similar to β/ω-TRTX-Tp2a (ProTx-II, PDB ID: 2n9t). ProTx-II is a spider venom peptide from the NaSpTx family 3 and a potent hNaᵥ1.7 inhibitor. In addition, sHsp1a did not contain defined beta sheets or alpha helices. Comparison of the backbone of sHsp1a to ProTx-II revealed that the loop between C16 to C21 and the loop between C2 to C9 were not aligned although the primary sequences of these regions are highly conserved; and ProTx-II contained a longer dynamic C terminus than sHsp1a. Moreover, sHsp1a lacked two important hydrophobic residues (L29 and W30) at the C-terminus, and used the residues Isoleucine (I) 27 and Leucine (L) 6 to replace the K27 and M6 in ProTx-II. This difference could explain why sHsp1a was insensitive to hNaᵥ1.5 at even 2 μM concentration. The art has shown that Methionine (M) 6, Tryptophan (W) 7, Arginine (R) 13, Valine (V) 20, Arginine (R) 22, Tryptophan (W) 24, Lysine (K) 27, Leucine (L) 29 and Tryptophan (W) 30 are the pharmacophore of ProTx-II to hNaᵥ1.5.

TABLE 1
Energies and structural statistics^{a} for
the final^{b} 20 structures of sHsp1a
Energies (kcal/mol)

| | |
|---|---|
| Overall | −825.6 ± 21.0 |
| Bonds | 14.3 ± 1.6 |
| Angles | 42.3 ± 3.4 |
| Improper | 19.8 ± 2.1 |
| Dihedral | 142.6 ± 2.1 |
| Van der Waals | −121.7 ± 6.7 |
| Electrostatic | −927.6 ± 23.7 |
| NOE | 0.12 ± 0.04 |
| Constrained dihedral (cDih) | 4.55 ± 1.0 |
| MolProbity statistics | |
| Clash man: (>0.4 Å/1000 atoms) | 16.7 ± 6.6 |
| Poor rotamers (%) | 0.4 ± 1.2 |
| Ramachandran outliers (%) | 0 ± 0 |
| Ramachandran favoured (%) | 9.77 ± 4.1 |
| MolProbity score | 1.88 ± 0.25 |
| MolProbity percentile | 80.6 ± 12.4^{c} |
| Atomic RMSD (Å) | |
| Mean global backbone (2-25)^{d} | 0.57 ± 0.17 |
| Mean global heavy (2-25) | 1.30 ± 0.19 |
| Mean global backbone (1-28) | 0.68 ± 0.16 |
| Mean global heavy (1-28) | 1.71 ± 0.27 |
| Distance restraints | |
| Intraresidue (i − j = 0) | 124 |
| Sequential (\|i − j\| = 1) | 135 |
| Medium range (\|i − j\| < 5) | 76 |
| Long range (\|i − j\| > 5) | 77 |
| Hydrogen bonds* | 2 |
| Total | 414 |
| Dihedral angle restraints | |
| ϕ | 20 |
| Ψ | 18 |
| χ1 | 2 |
| Total | 40 |
| Violations from experimental restraints | |
| Total NOE violations exceeding 0.3 Å | 0 |
| Total dihedral violations exceeding 3° | 2 |

^{a}±St Dev
^{b}Base on structures with highest overall MolProbity score (Davis et al., 2007).
^{c}100^{th} percentile is the best amoung structures of comparable resolution; 0^{th} percentile is the worst.
^{d}RMSD calculated in MOLMOL (Koradi et al., 1996).
^{e}Two restraints were used per hydrogen bond.

### Example 4: Hsp1a is a Potent Inhibitor of the Human Naᵥ1.7 Sodium Channel Subtype (hNaᵥ1.7

Hsp1a is More Potent on hNaᵥ1.7 than the Other NaV Subtypes

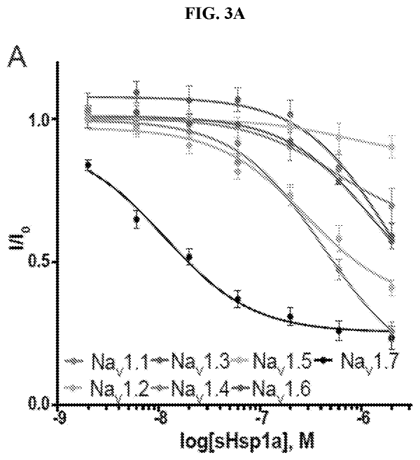

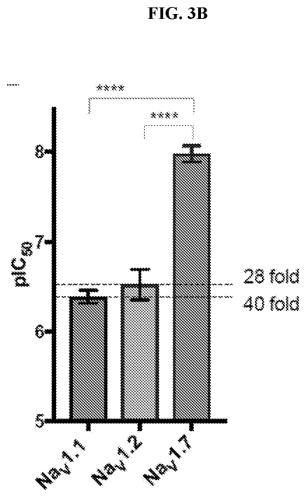

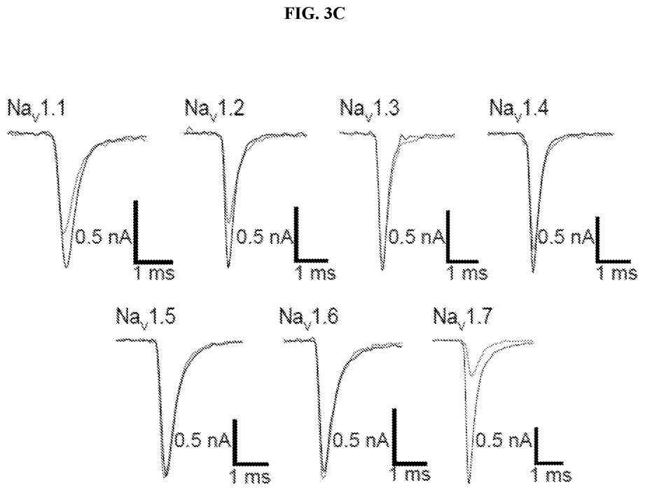

To determine the selectivity of Hsp1a on different human Naᵥ isomers, sHsp1a was screened by automatic whole cell patch clamp (QPatch 16X) against hNaᵥ1.1, hNaᵥ1.2, hNaᵥ1.3, hNaᵥ1.4, hNaᵥ1.5, hNaᵥ1.6 or hNaᵥ1.7. FIG. 3A, C show that sHsp1a has the same efficacy on both hNaᵥ1.1 and hNaᵥ1.7 at 2 μM (current inhibition 75%). However, sHsp1a potently inhibited hNaᵥ1.7 (pIC50 7.98±0.09 M, mean±S.E.M) with 40 fold selective over hNaᵥ1.1 (pIC50 6.39±0.07 M), 28 fold selective over hNaᵥ1.2 (max. current inhibition 60% at 2 μM, pIC50 6.52±0.17 M) and more than 100 fold selective over hNaᵥ1.3-hNaᵥ1.6 (max. current inhibition<50% at 2 μM, pIC50<6 M) (FIG. 3B). The inhibition activity of sHsp1a on hNaᵥ1.7 started to saturate at 200 nM (FIG. 3A). At this concentration, sHsp1a blocked 70% of the hNaᵥ1.7 currents while it only blocked 30% of hNaᵥ1.1 and hNaᵥ1.2 currents and did not show any significant inhibition on hNaᵥ1.3 to hNaᵥ1.6 currents (FIG. 3C).

Analysis of the native Hsp1a and rHsp1a showed that sHsp1a had very similar physiological activity as the native Hsp1a. There is no significant pIC50s difference between the native Hsp1a and sHsp1a FIG. 1C-E. However, rHsp1a, which lacks C-terminus amidation, showed significant decreased inhibitory activity on hNaᵥ1.7 and its pIC50 was more than 20 fold less than pIC50s of sHsp1a and native Hsp1a (FIG. 1D).

sHsp1a is a Gating Modifier Toxin that Alters the Steady-State Inactivation of hNaᵥ1.7.

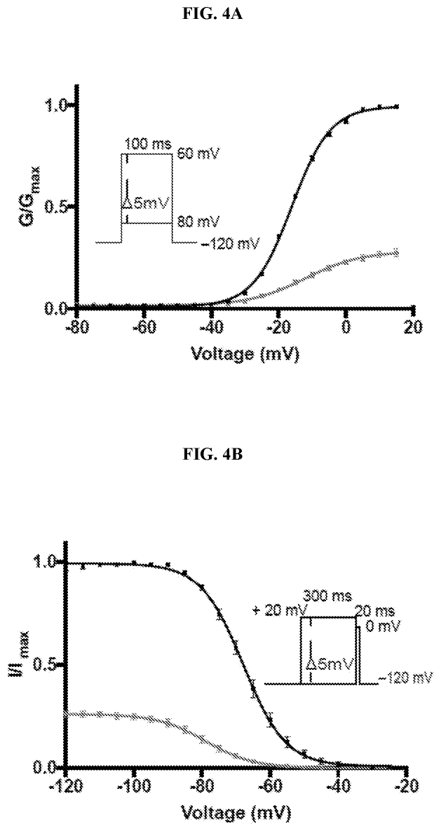

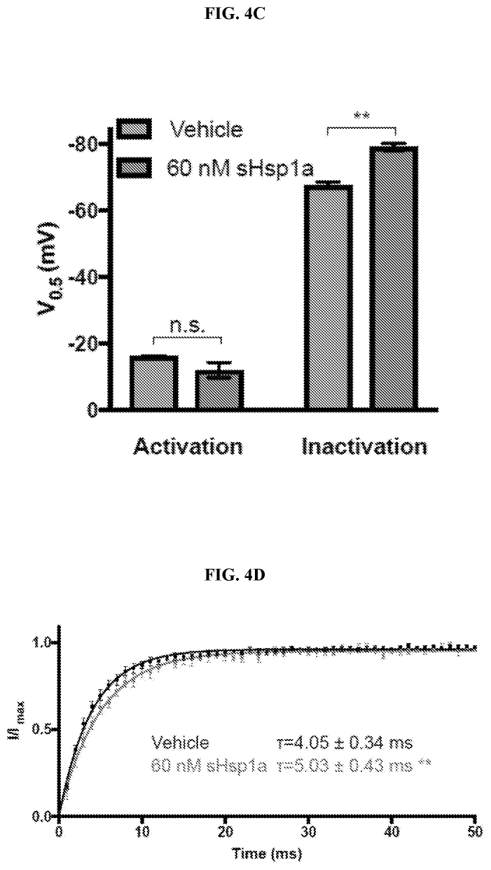

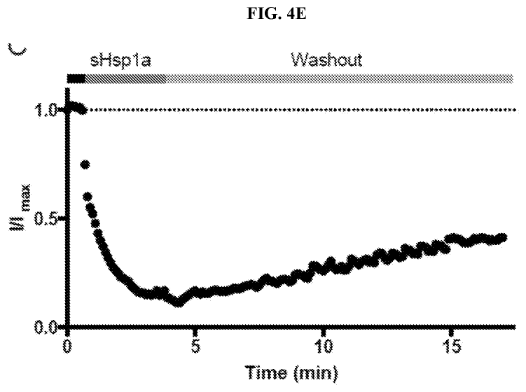

The kinetic studies of sHsp1a on hNaᵥ1.7 were conducted by a conventional whole cell patch clamp. As shown in FIG. 4A, C, the current-voltage (I-V) relationship of the steady state inactivation shows that the V_{0.5} of inactivation significantly shifted to the hyperpolarisation state after addition of a non-saturating concentration of sHsp1a (60 nM) (V_{0.5} inactivation Δ=−11.62 mV; paired t test compared to vehicle, P<0.01). The conductance-voltage (G-V) relationships showed no significant shift in the V_{0.5} of activation after addition of 60 nM sHsp1a (paired test compared to vehicle, P>0.05) FIG. 4A, C. Moreover, 60 nM of sHsp1a slowed the recovery from fast inactivation (Δτ=1 ms; paired t test compared to vehicle, P<0.01; FIG. 4D). sHsp1a behaved as a reversible gating modifier peptide to hNaᵥ1.7 because sHsp1a-induced currents slowly recovered in ˜10 minutes following washout in peptide-free solution (FIG. 4E). Together these data suggested that sHsp1a was a gating modifier peptide and might allosterically modulate the hNaᵥ1.7 channel by targeting the Voltage-Sensing Domain. Consistent with these pharmacological characteristics, Hsp1a shared 84% similarity with β/ω-TRTX-Tp2a (ProTx II) and β-TRTX-Gr1b (GsAFI), two potent and selective hNaᵥ1.7 gating modifiers from the NaSpTx family 3. The IC₅₀ of ProTx II and GsAFI were respectively 0.3 nM and 40 nM.

### Example 5: Hsp1a Exhibited High Stability in Human Serum

To determine the efficacy of Hsp1a for in vivo testing, the biological stability of Hsp1a in human plasma was assessed. Hsp1a was incubated in human serum at 37° C. for up to 24 hours. The ICK peptide, ω-conotoxin MVIIA (MVIIA) and the very unstable peptide, human Atrial Natriuretic Peptide (hANP) were used as controls. As shown in FIG. 5A-B, the amount of sHsp1a remained constant after 24 hours. In contrast, only 50% of the initial MVIIA concentration remained after 24 hours and hANP was degraded in the first 1-2 hours of incubation. FIG. 5B showed that sHsp1a was unaffected in human plasma up to 24 hours. This high stability of sHsp1a in human serum further suggested that the efficacy of Hsp1a would not be hampered by human plasma and Hsp1a would have time to migrate to pharmaceutical targets in human before degrading.

### Example 6: Synthetic Hsp1a (sHsp1a) Decreased the Pseudo-Related Pain Responses to Colorectal Distension in the Chronic Visceral Hypersensitivity (CVH) Animal Model In Vivo

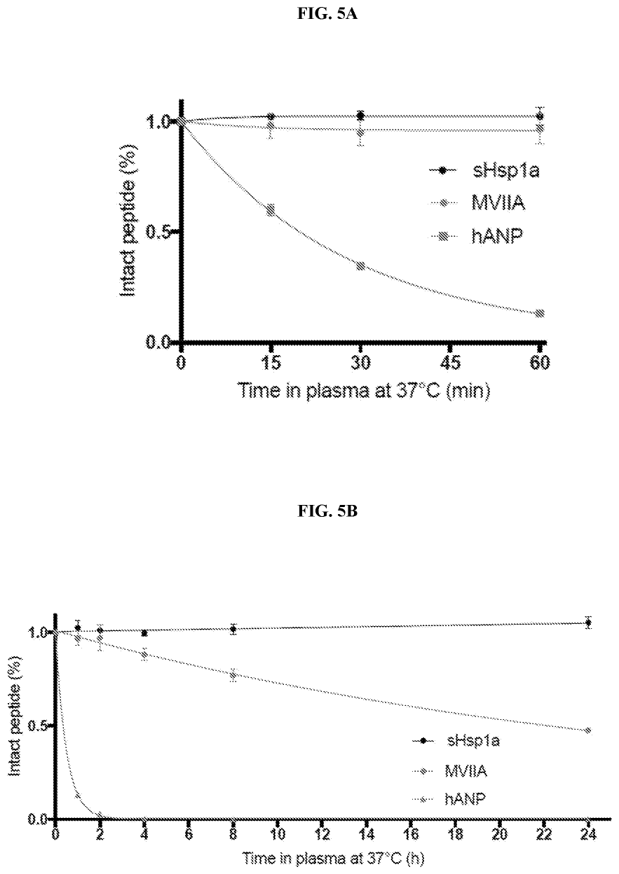

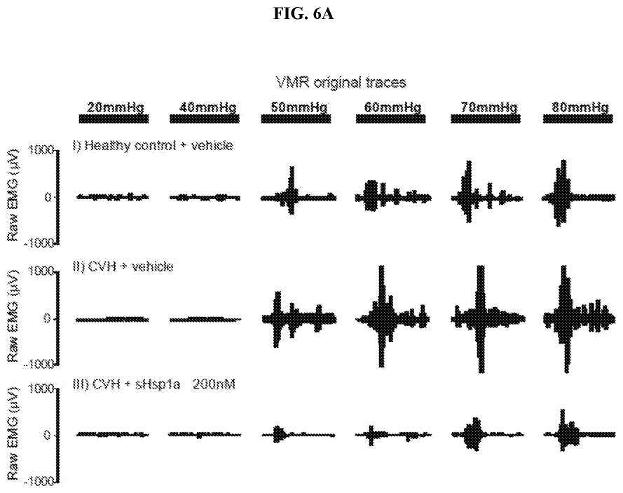

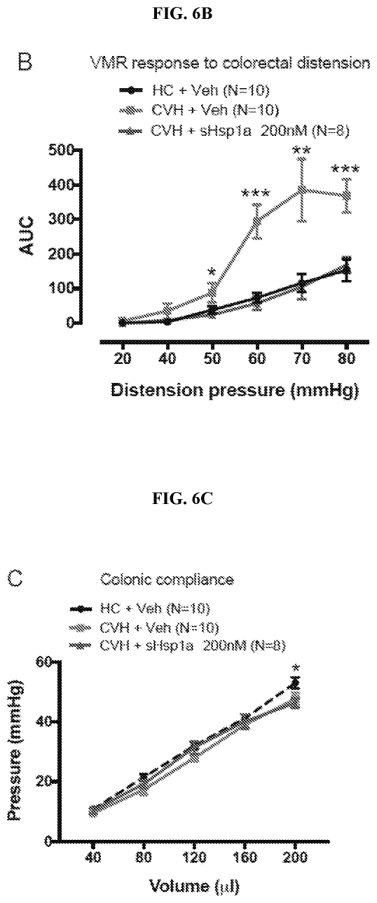

To determine whether Hsp1a could be used for the treatment of pain, the effect of Hsp1a in a mouse model of chronic visceral hypersensitivity (CVH) was assessed. In this model, the pseudo-related pain response was triggered by a noxious distension of the colorectum that caused a nociceptive brainstem reflex generated by the contraction of the abdominal muscles (the visceromotor response (VMR). As shown in FIG. 6A-B, CVH animals treated with vehicle (CVH+Veh) exhibited hyperalgesia, characterized by an increased sensitivity to noxious distensions (>50 mmHg). Intracolonic treatment with sHsp1a (200 nM) significantly reduced the VMR to colorectal distension comparing to vehicle treated animals. (Responses were normalized to healthy levels; FIG. 6A-B). Colonic compliance did not change considerably in CVH mice treated with either vehicle or sHsp1a (FIG. 6C), suggesting that changes in the VMR to colorectal distension were not due to variations in smooth muscle function. These data indicated that sHsp1a-mediated inhibition of the Naᵥ1.7 channel would be a valuable strategy for treating pain in irritable bowel syndrome patients and other chronic visceral pain diseases.

### Example: 7 Hsp1a Did Not Cause Acute Toxicity When Injected Intravenously in Healthy Mice

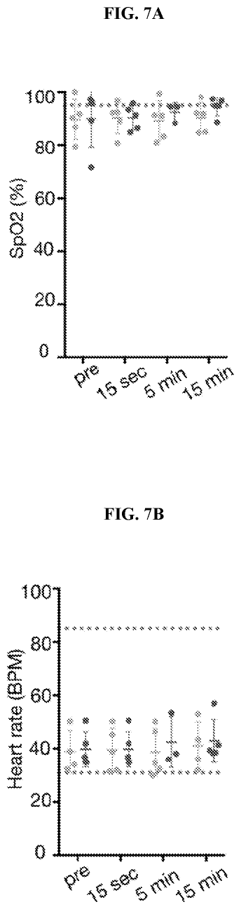

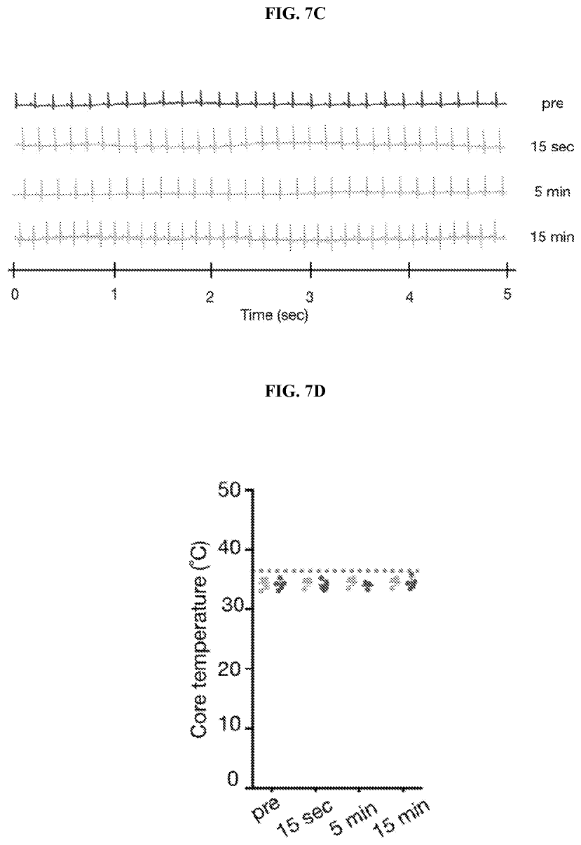

To demonstrate that Hsp1a had no effect on the respiratory musculature, such as diaphragm and thoracic muscles, oxygen saturation in the peripheral blood was monitored. Mice were anesthetized with 68 μM isoflurane and placed on a heated pad. Mice were injected with 7 nmol Hsp1a-FL in 100 μL of PBS through a catheter placed into the tail vein. Oxygen saturation was monitored by placing a clip sensor on the animal's left thigh before, 15 seconds, 5 minutes and 15 minutes after injection. Control animals were injected with PBS. As shown in FIG. 7A, the average percentage of oxygen saturation (SpO2) in mice was 89.7 (SD=7.5) pre-injection and it was 90.3 (SD=6.0) 15 seconds post injection, 89.2 (SD=7.4), 5 minutes post injection, and 90.3 (SD=5.6) 15 minutes post injection. No statistical significance was observed between pre-injection and the post-injection data points (p=0.62, >0.99 and 0.81, respectively). Moreover, no statistical significance was observed between mice injected with Hsp1a and the control group (p=0.84 for pre-injection, >0.99 for 15 seconds post injection, 0.57 for 5 min post injection and 0.31 for 15 min post injection).

To demonstrate that Hsp1a had no effect in the heart muscle, electrocardiogram trace of heart muscles were monitored before and up to 16 minutes after injection. No changes were observed on the EKG pattern suggesting that the electrical activity in the heart remained the same before and after injection (FIG. 7B-C). Heart rate were continually monitored pre and post-injection (15 seconds, 5 and 15 minutes), along with controls. The average of heart rate before injection was 388 (SD=83). This result was not significantly different between the values observed in the different time points after injection (395, SD=82, p=0.88 for 15 seconds; 387, SD=89, p=0.63 for 5 min; and 409, SD=91, p=0.99 for 15 minutes). No statically significant differences were observed when comparing heart beat values in the Hsp1a and control groups (p=0.56 in the pre-injection group, p=0.84 in the 15 seconds after injection group, p=0.39 in the 5 minutes after injection group, and p=0.73 in the 15 minutes injection group).

Mouse body temperatures were also monitored in parallel. In particular, the heated platform was constantly kept at 39° C. The average core temperature of mice before Hsp1a injection was 34.3° C. (SD=0.9). No difference in core temperature was seen in any of the timepoints after injection (34.5° C., SD=0.63, p=0.63 15 seconds post injection, 34.6° C., SD=0.66, p>0.99 5 min post-injection and 34.6° C., SD=0.64, p>0.99 15 minutes post-injection) (FIG. 7D). No statistical significance was observed when comparing the Hsp1a injected group with controls (p>0.99 pre-injection, p=0.42 15 seconds post-injection, p=0.57 5 minutes post-injection, and p=0.55 15 minutes post-injection).

### Example 8: Naᵥ1.7 Expression in the Peripheral Nervous System

Naᵥ1.7 Biomarker Validation in Human Vagus Nerves

To determine the expression pattern of the Naᵥ1.7 channel, Naᵥ1.7 expression in human vagus nerves from the cervical region was determined. All nerves were donated by the Fusion Solution Bioskills Laboratory autopsy program. The biospecimens were about 5 cm long (n=10 nerves from n=5 individuals) and frozen using optimal cutting temperature (OCT) compound directly after surgical resection, and sectioned at 10 μm thickness for staining. Staining with an anti-Naᵥ1.7 antibody staining showed that Naᵥ1.7 expressed throughout the axons. The specificity of the antibody was confirmed with an isotype control staining. Hematoxylin & Eosin (H&E) staining was used to visualize the nerve structure, particularly, the nerve axons and Schwann cells. Together these data confirm that the Naᵥ1.7 channel was expressed in peripheral neurons.

Naᵥ1.7 Expression in Mouse Sciatic Nerves.

To evaluate the potential of Naᵥ1.7 as a biomarker target for imaging the peripheral nervous system, the Naᵥ1.7 content in the sciatic nerve of female athymic nude mice was examined. Similar to the human vagus nerves, the sciatic nerves were surgically removed and embedded in OCT, sectioned at 10 μm thickness and immunohistochemically stained with anti-Naᵥ1.7 antibody. Consistent with the data obtained for human peripheral nerves, staining highlighted the axonal bundles. H&E staining of adjacent slides highlighted the Schwann cells within the nerve. No staining was observed when using isotype control antibodies, confirming specificity.

### Example 10: Chemical Synthesis of Fluorescently Labeled Hsp1a

Modification of Hsp1a Via Nucleophilic Substitution

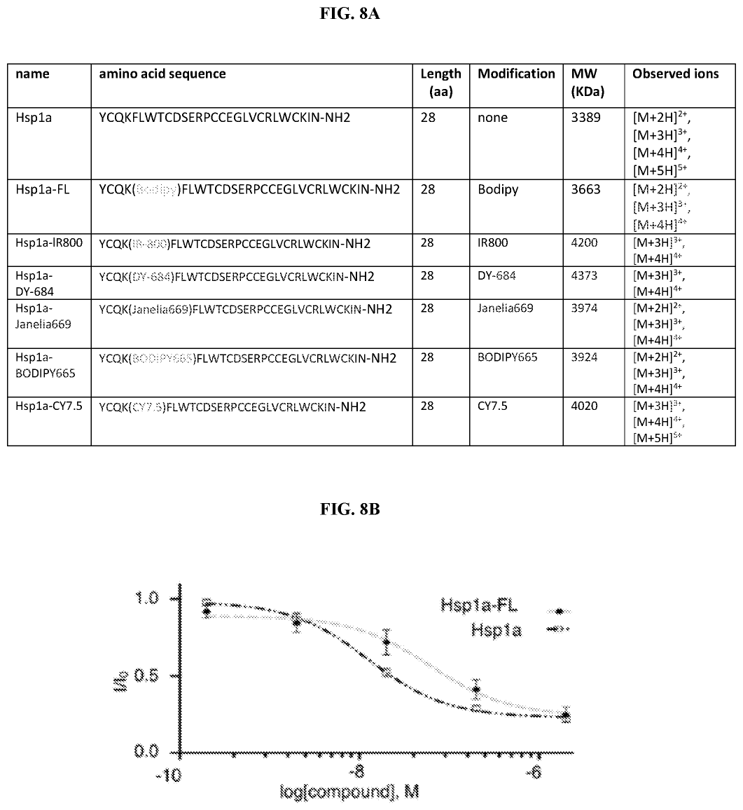

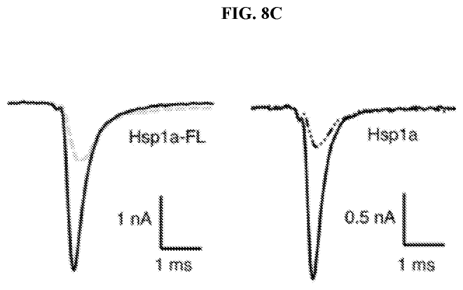

Hsp1a was modified with BODIPY-FL due to the small size and relatively high stability of this fluorophore (FIG. 8A). The chemical transformation was performed under basic conditions in a mixture of water and acetonitrile, and produced Hsp1a-FL in 55% yield and 83% purity. The retention time (rₜ) shifted from 21 min for Hsp1a to 24 min for Hsp1a-FL. The major impurity was the partially reduced peptide, 9% (rₜ 24.2 min), which was also present in the starting material (rₜ 21 min, 90%; and rₜ 21.2 min, 10% for Hsp1a and reduced Hsp1a, respectively). LC/MS spectra for both Hsp1a and Hsp1a-FL showed clean peak families confirming the peptides' calculated masses, 3389 and 3663 Da for Hsp1a and Hsp1a-FL, respectively. Hsp1a was also modified with IR800, DY-684, Janelia669, BODIFY665, and CY7.5 (FIG. 8A).

Hsp1a could have been modified at three principal nucleophilic positions (the N-terminal amine, K4 and K26). To determine the location of the BODIPY-FL conjugation, a tryptic digest was performed. For the unmodified Hsp1a, ions that correspond to the fragment F5-R22 (2114.90 Da) was identified. This fragment was not seen for digested Hsp1a-FL. The digestion of Hsp1a-FL gave rise to the novel fragment Y1-R22 plus the mass of the BODIPY scaffold without BF2 (2864.24 Da). Observation of this peak suggested that conjugation occurred at K4 because if the fluorophore had not been conjugated at K4, the fragment would have been digested by trypsin into two smaller fragments. However, a small amount of fluorescent side products was identified, including uncharacterized Hsp1a with two conjugated dyes. The data suggested that the predominant modification of Hsp1a at K4 was likely electronically or sterically favored.

To determine whether fluorescent modification of Hsp1a affected its potency toward Naᵥ1.7, the inhibition of human Naᵥ1.7 by Hsp1a and Hsp1a-FL using automated whole-cell patch-clamp electrophysiology. As shown in FIG. 8B-C, Hsp1a was an extremely potent inhibitor of Naᵥ1.7 (IC₅₀=13 nM), and its potency is only marginally reduced by addition of the BODIPY-FL label in Hsp1a-FL (IC₅₀=62 nM). As such, fluorescent modification of Hsp1a marginally reduces its potency, but it remains a potent nanomolar inhibitor of Naᵥ1.7 that should be suitable for targeting peripheral neurons.

Histological Validation of Hsp1a-FL Uptake in Mouse Sciatic Nerves.

Histological analysis was performed in mice to correlate the mesoscopic and cellular distribution of Hsp1a-FL in peripheral nerves. Mice were injected with Hsp1a-FL (7 nmol, 68 μM in 100 μL of PBS), sacrificed after 30 min, and then their sciatic nerves were removed and sectioned for imaging. Fluorescence signal was obtained in the green channel (488 nm excitation), showing circular patterns when nerves were sliced transversely, and linear patterns when nerves were sliced longitudinally. Both patterns resembled the tubular structure of nerve bundles and axons. In order to visualize the epineurium and perineurium, as well as more centrally showing endoneurium and nerve bundles, longitudinal sections were performed close to the surface. In addition, adjacent H&E and anti-Naᵥ1.7 staining, were consistent with the histological structures obtained with Hsp1a-FL (center and right column for H&E and anti-Naᵥ1.7, respectively). The specificity of Hsp1a-FL staining was determined by coinjecting a 3-fold excess of the original, unmodified peptide (7 nmol, 68 μM in 100 μL of PBS for Hsp1a-FL and 21 nmol, 210 μM in 100 μL for Hsp1a). The coinjection of a molar excess of unlabeled Hsp1a prevented uptake of Hsp1a-FL in sciatic nerves, suggesting that the probe was specific and its target saturable.

Rapid and Selective Accumulation of Hsp1a-FL in Mouse Sciatic Nerves.

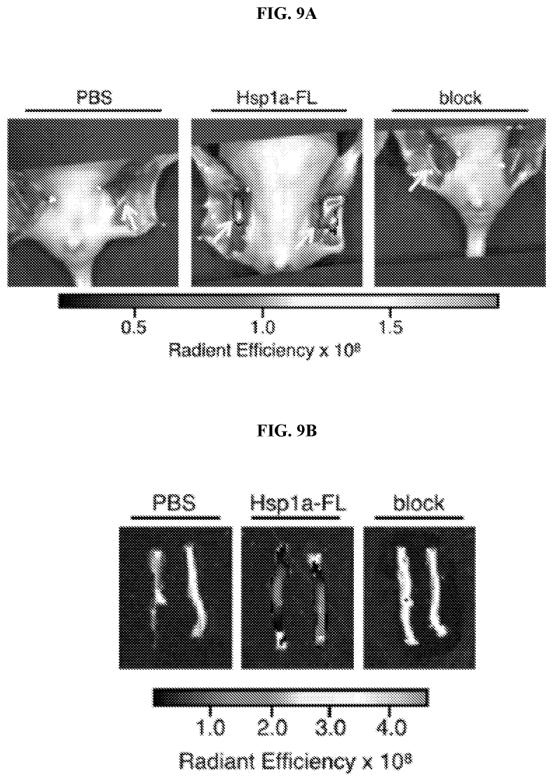

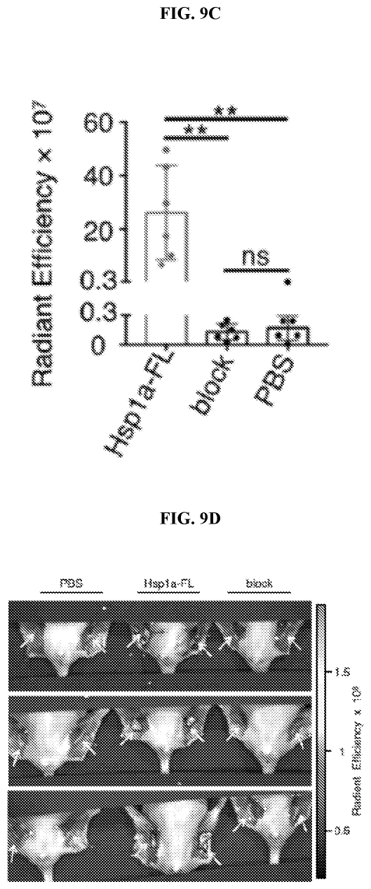

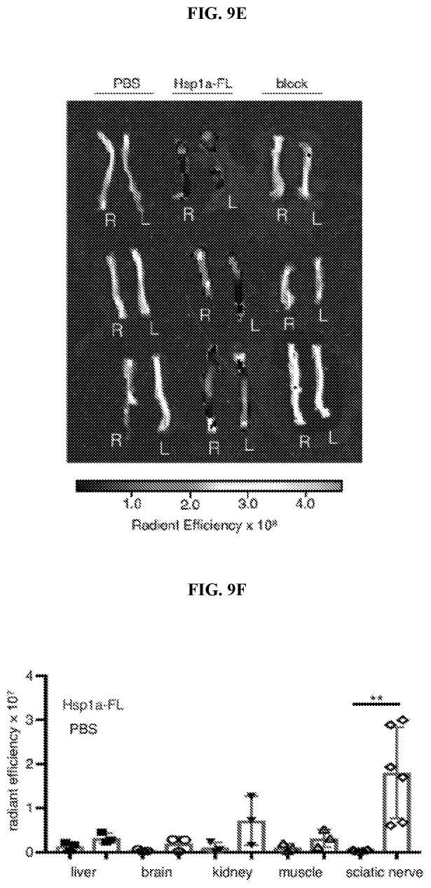

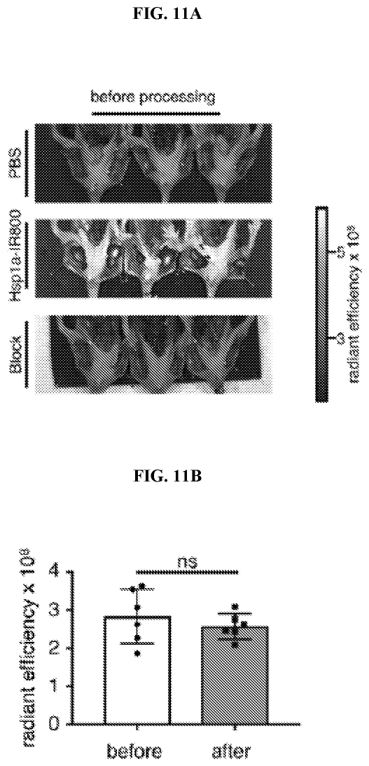

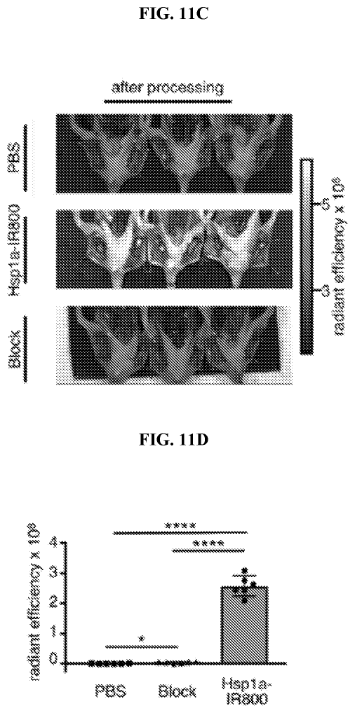

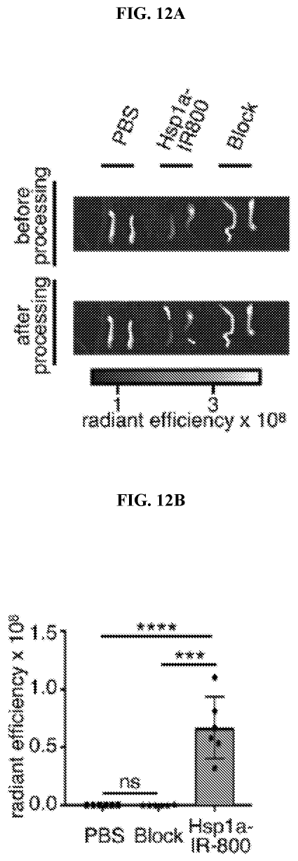

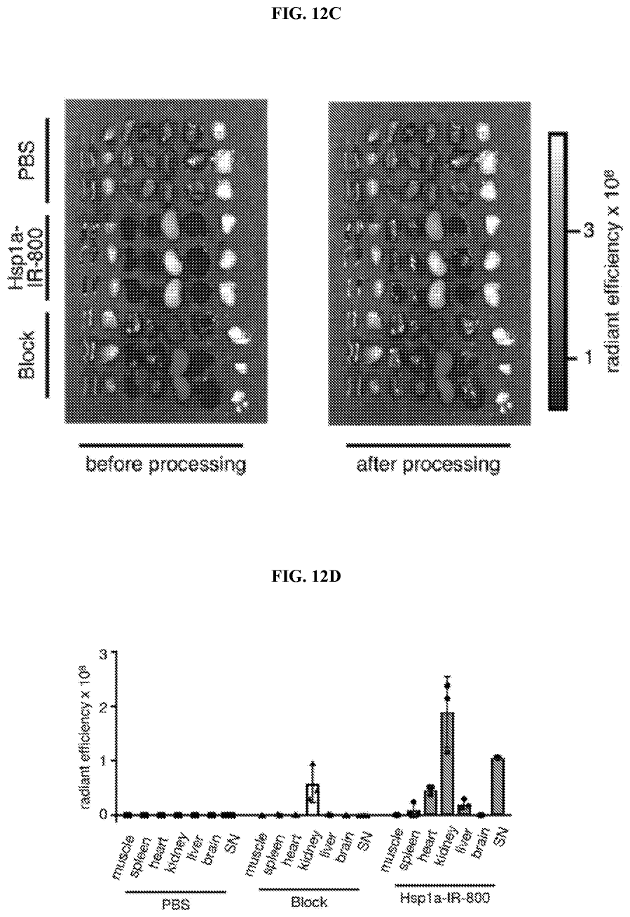

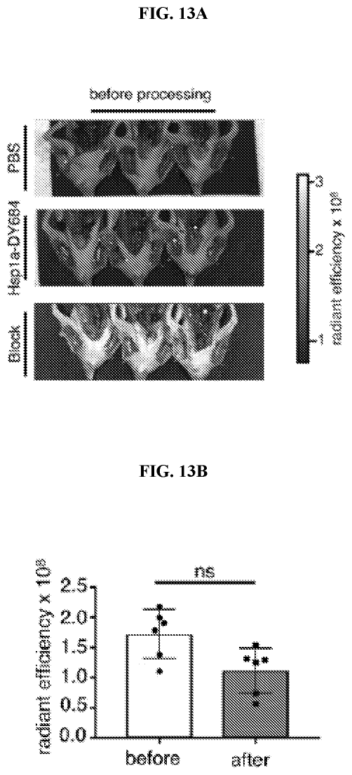

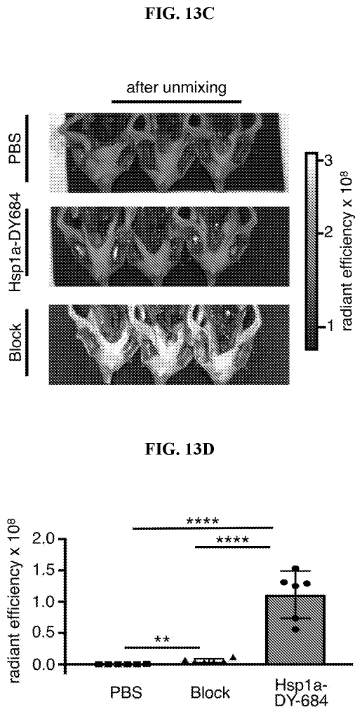

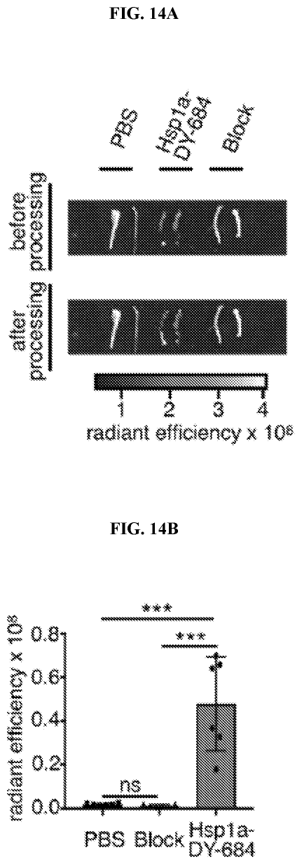

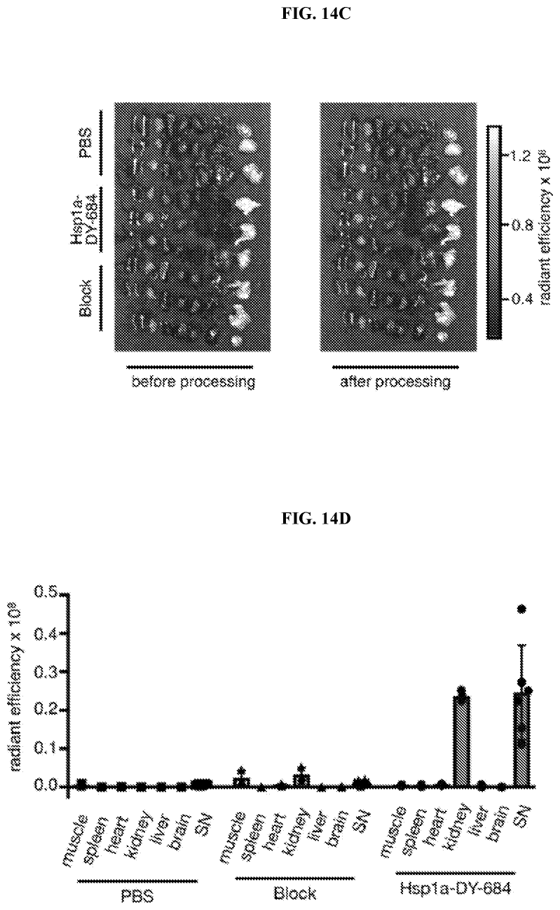

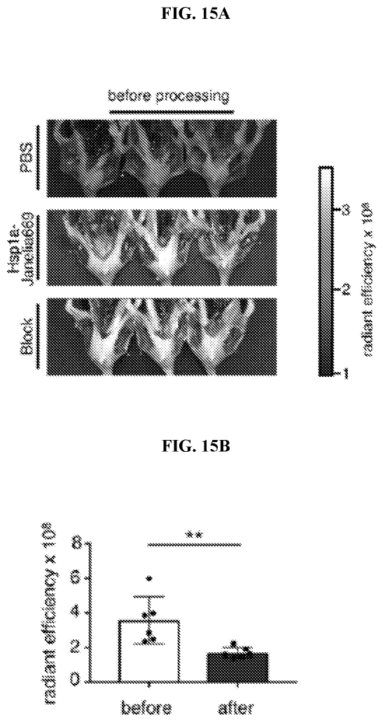

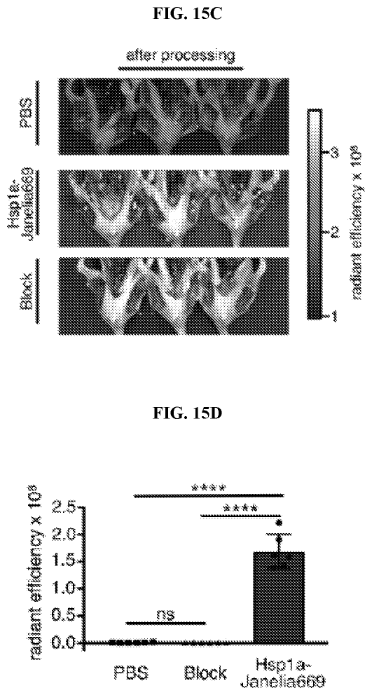

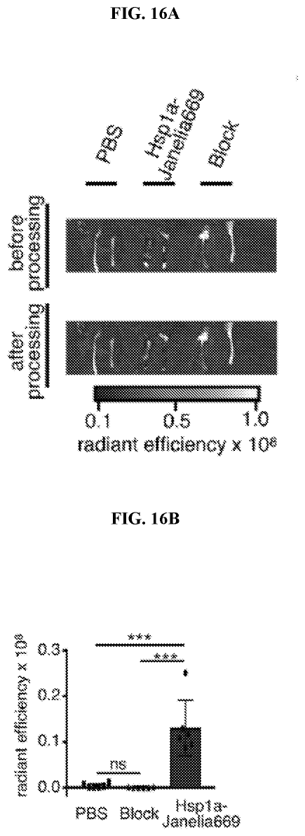

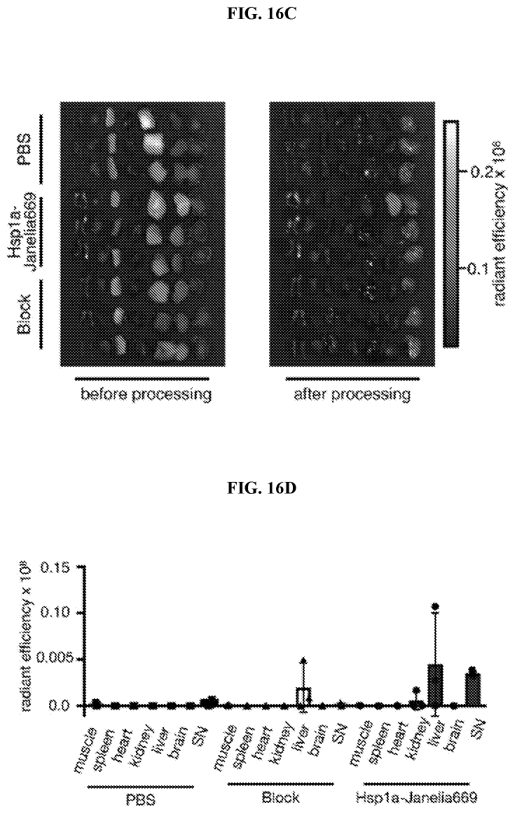

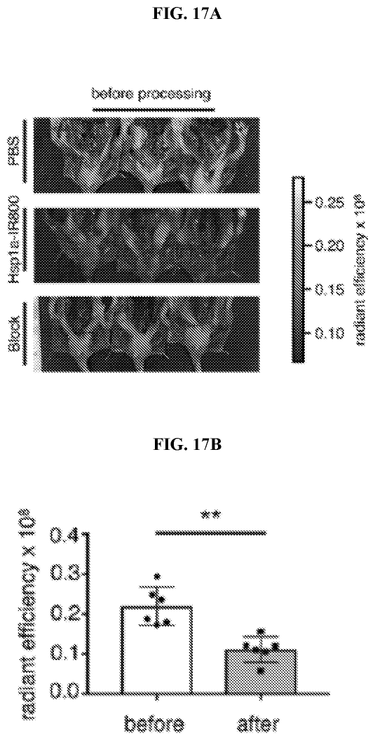

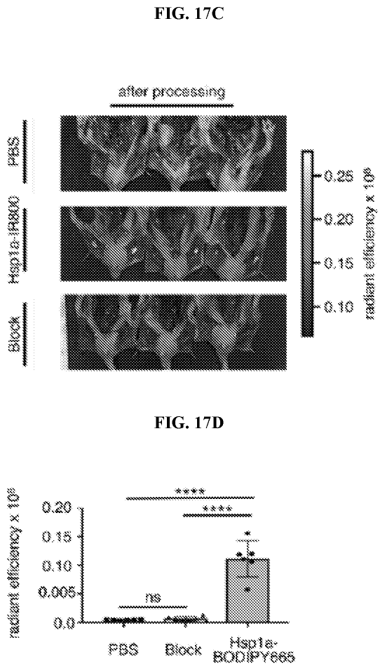

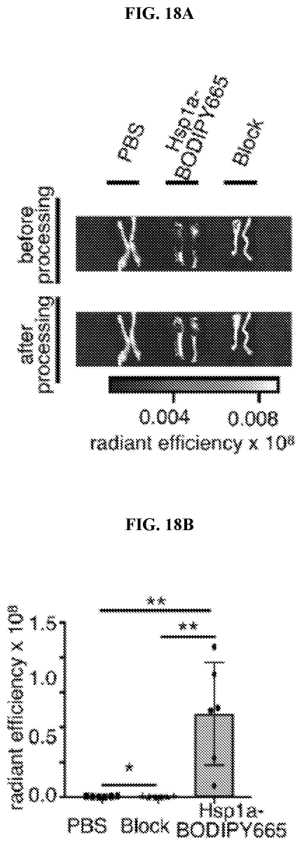

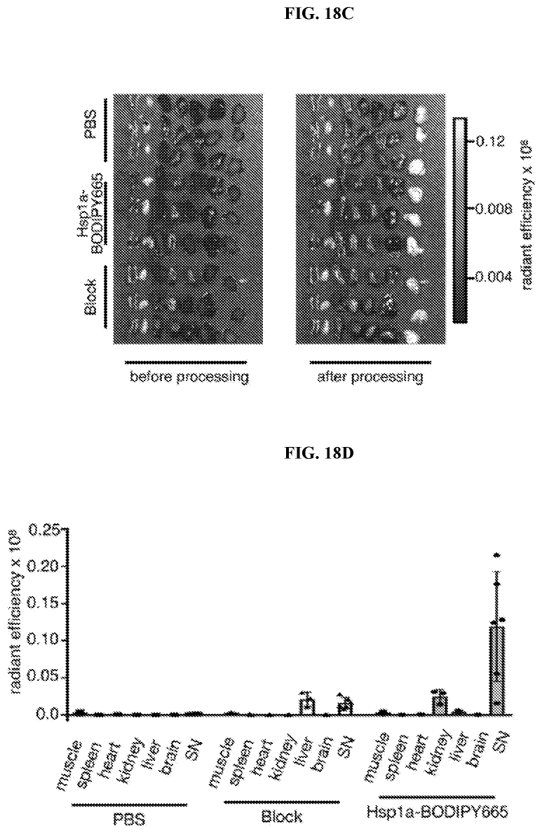

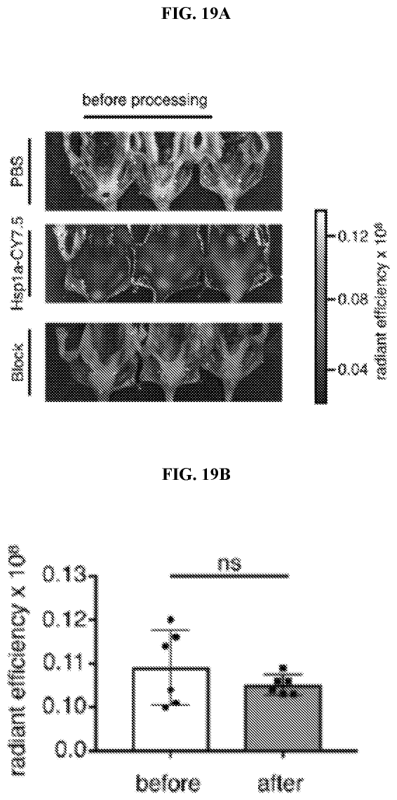

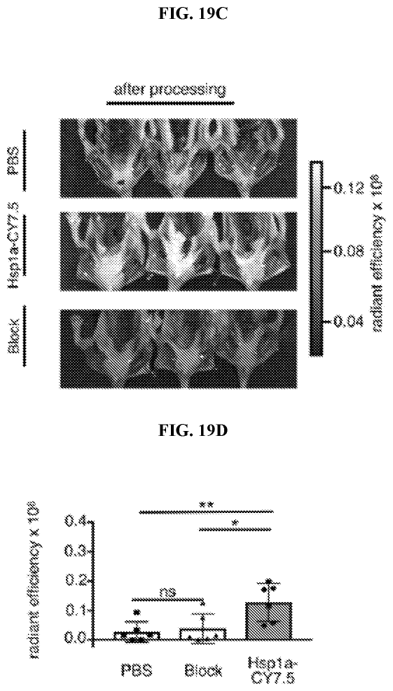

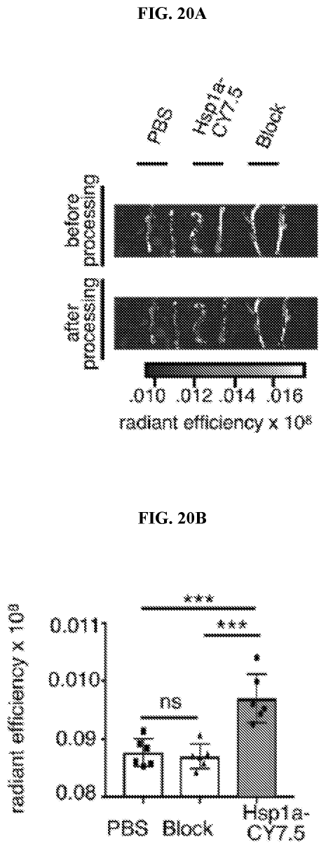

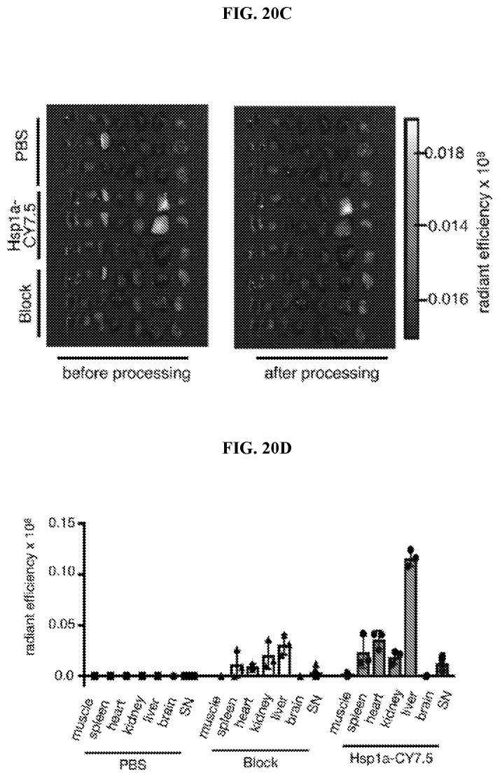

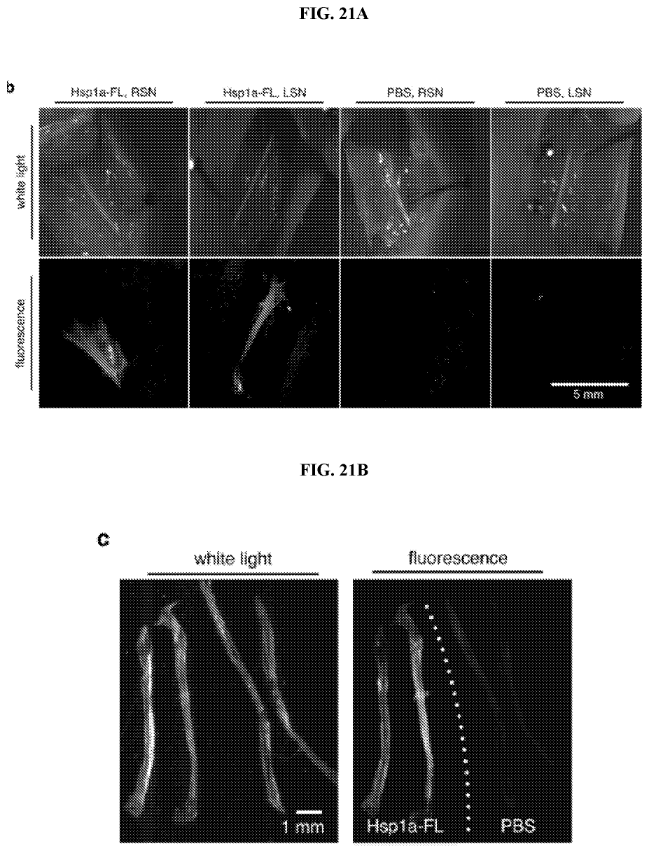

To assess the accumulation of Hsp1a-FL in fresh unprocessed peripheral neurons, mice were injected intravenously with Hsp1a-FL alone (7 nmol, 68 μM in 100 μL of PBS) or in combination with an excess of unmodified peptide (21 nmol, 204 μM in 100 μL of PBS) (block). Mice were then sacrificed 30 min post-injection. Their right and left sciatic nerves (RSN and LSN) were exposed (FIG. 9A, D) and imaged using epifluorescence imaging performed using an IVIS Spectrum in vivo imaging system (excitation 465/30 nm; emission 520-580 nm). In mice receiving just the imaging agent, the sciatic nerves were clearly visible, whereas uptake was significantly reduced in mice that received the imaging agent in combination with excess unmodified peptide, (radiant efficiency: (6.3±3.2)×107 and (0.03±0.01)×107 for Hsp1a-FL and coinjection (blocking), respectively; Student's unpaired t-test, P<0.001, FIG. 9A, D). Similar results were obtained with similarly other dyes (FIGS. 11-20).

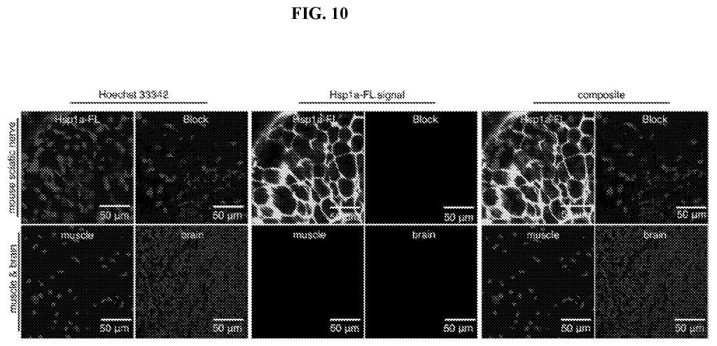

As shown in FIG. 9B-C, E, Ex vivo, high fluorescence intensities (due to dye accumulation) were only observed in sciatic nerves injected with Hsp1a-FL alone. In mice receiving the imaging agent only, the sciatic nerves were clearly visible, with a mean radiant efficiency of (26±0.13)×10⁷ (FIG. 9A), whereas mice receiving the imaging agent in combination with the unmodified peptide (21 nmol, 204 μM in 100 μL of PBS) had a statistically significant 200-fold reduction in radiant efficiency to (0.13±0.08)×10′. (Student's unpaired t-test, P<0.05). While quantitative assessment with fluorophores across organ systems was not feasible, particularly in the visible range, kidneys exhibited higher fluorescence signals. The radiant efficiency for the kidneys was 0.1±0.01×10⁷ and 0.71±0.06×10⁷ with and without injection, respectively; Student's unpaired t test, P=0.14, FIG. 9F). No significant fluorescent signal was observed in other organs, including muscle and liver when comparing animals injected with Hsp1a-FL and PBS (FIG. 9F). Examination of fresh tissue under a confocal microscope revealed nerve patterns that were similar to those observed with histological staining (FIG. 10). Similar results were obtained with other dyes (FIGS. 11-20).

### Example 11: Hsp1a Imaging with Surgical Microscope

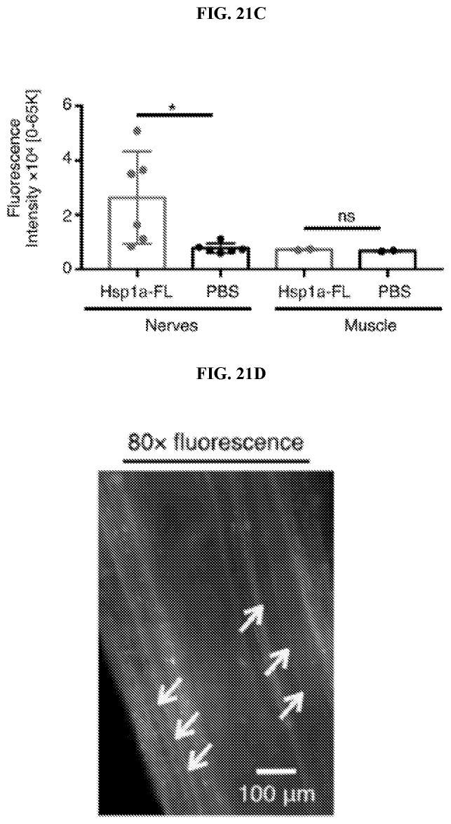

To confirm the feasibility of imaging in a more clinically relevant setting, Hsp1a-FL with a Lumar surgical fluorescence stereoscope (SteREO Lumar v12, Zeiss, Jena, Germany). Mice were injected with Hsp1a-FL (7 nmol, 68 μM in 100 μL of PBS) or 100 μL of PBS. 30 min post-injection, mice were sacrificed and their sciatic nerves exposed for imaging. FIG. 21A shows images of sciatic nerves obtained under white light imaging conditions (top row) and Hsp1a-FL fluorescence (bottom row; FIG. 21E) for both mice injected with Hsp1a-FL and PBS. Unlike the IVIS Spectrum, the Lumar Fluorescence imaging system does not provide image deconvolution, therefore no support for autofluorescence correction. Strong specific fluorescence in nerves of animals injected with Hsp1a-FL were observed. The fluorescence intensity of Hsp1a-FL-injected mice (2.6±1.7)×10⁴ was significantly lower than that of PBS-injected mice ((0.8±0.2)×10⁴ (Mann-Whitney's test, P<0.05, FIG. 21B-C). At higher magnification, the Lumar fluorescence stereoscope revealed structures within the nerves that resemble the tubular and axonal features observed on nerve sections during confocal imaging. These data provided additional support for Hsp1a-FL being highly specific for Naᵥ1.7. Together, the combination of the Lumar stereoscope and Hsp1a-FL could thus be used clinically for intraoperative, contemporaneous mapping of peripheral nerves.

### Example 12: Fluorescence Labeling of Hs1a for Near-Infrared Nerve Visualization

Selectivity of Hs1a Across Ion Channels Subtypes

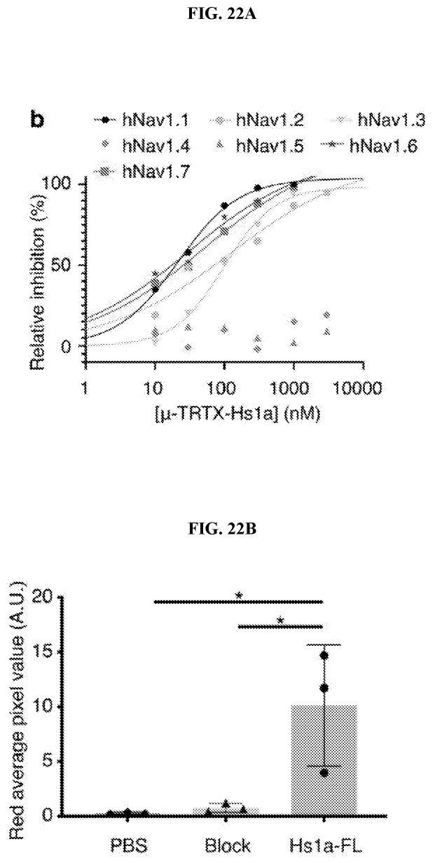

Hs1a was isolated from the venom of the Chinese bird spider, Haplopelma schmidti. To assess the selectivity of Hs1a for Naᵥ channel subtypes, Hs1a was tested on HEK cells stably transfected with human Naᵥ1.1-Naᵥ1.7 channels using automated patch clamp techniques. Hs1a was shown to have affinity for Naᵥ1.1, Naᵥ1.2, Naᵥ1.3, Naᵥ1.6 and Naᵥ1.7 channels with IC₅₀ values in the low nanomolar ranges (FIG. 22A). In particular, the IC₅₀ for Naᵥ1.1, Naᵥ1.2, Naᵥ1.3, Naᵥ1.6 and Naᵥ1.7 was 19.4, 82, 107, 19.2, 26.9 nM respectively. However, Hs1a didn't show any affinity for Naᵥ1.4 and Naᵥ1.5 subunit at concentrations up to 3 μM (FIG. 22A). The IC₅₀ value for hNaᵥ1.7 obtained in mammalian cells correlates with the IC₅₀ obtained by two-electrode voltage clamp on hNaᵥ1.7 expressed in X. laevis oocytes. The selectivity of Hs1a for off-target voltage-gated calcium and potassium channels were also tested. As shown in Table 2, Hs1a did not inhibit voltage-gated calcium and potassium channels.

TABLE 2
Hs1a affinity for Naᵥ channels stably expressed on
the membranes of HEK293 cells
Hs1a affinity for Naᵥ channels stably expressed
on the membranes of HEK293 cells

| ION | SUB- | | |
|---|---|---|---|
| CHANNEL | TYPE | IC₅₀ | FEATURE |
| NAᵥ | Naᵥ1.1 | 19.4 nM | ganglia |
| | Naᵥ1.2 | 82.2 nM | unmyelinated |
| | | | neurons, ganglia |
| | Naᵥ1.3 | 106.8 nM | mostly fetal |
| | | | nervous system |
| | Naᵥ1.4 | >3000 | adult neuro-muscular |
| | | | junction |
| | Naᵥ1.5 | >3000 | developing SM and |
| | | | cardiac muscle |
| | Naᵥ1.6 | 168 nM | axons |
| | Naᵥ1.7 | 45.7 nM | axons |
| | Naᵥ1.8 | >3000 | axons |
| | Naᵥ1.9 | >3000 | axons |
| CAᵥ | Caᵥ1.1 | >3000 | — |
| | Caᵥ1.3 | >3000 | cardiac muscle |
| | Caᵥ2.2 | >3000 | — |
| Kᵥ | K_{v2·}1 | >3000 | — |
| | hERG | >3000 | — |

Design of the Fluorescence Peptide, Hs1a-FL

To design a fluorescent Hs1a peptide synthesize Hs1a-FL for use as a biomarker for intraoperative applications, a fluorophore with near-infrared (NIR) emission spectrum and with favorable tissue penetration potential for intraoperative applications was chosen Table 3. In particular, Cy7.5 fluorescent dye was chosen for nerve imaging. Hs1a was then modified via nucleophilic substitution as previously described in Example 10. The synthesis was performed under basic conditions in a mixture of water and acetonitrile, with 14% yield. The retention time (rₜ) shifted from 12 min for the unmodified Hs1a to 16 min for Hs1a-FL. The major impurities were characterized as the partially reduced peptide, 3% (rₜ 16.2 min), which was also present in the starting material (rₜ 12 min, 80% and rₜ 12.2 min, 20% for Hs1a and reduced Hs1a, respectively). LC/MS spectra for both Hs1a and Hs1a-FL showed clean peak families confirming the peptides' calculated masses of 3850.74 Da and 4482.12 Da for Hs1a and Hs1a-FL, respectively (Table 3). In addition, fluorescence of 0.1 μM Hs1a peptide and 0.1 μM Hs1a-FL were collected to confirm dye conjugation.

TABLE 3
Details of Hs1a and Hs1a-FL
Details of Hs1a and Hs1a-FL

| | | LENGTH | | MW |
|---|---|---|---|---|
| NAME | AMINO ACID SEQUENCE | (AA) | DYE | (KDA) |
| HS1A | GNDCLGFWSACNPKNDKCC | 35 | None | 3850.74 |
| | ANLVCSSKHKWCKGKL | | | |
| | (SEQ ID NO: 2) | | | |
| HS1A-FL | GNDCLGFWSACNPK | 35 | Cy7.5 | 4482.12 |
| | (Cy7.5)NDKCCANLVCSS | | | |
| | KHKWCKGKL(SEQ ID | | | |
| | NO: 13) | | | |

Histology and Hs1a-FL Imaging of Mouse Sciatic Nerve

To assess the possibility of using Hs1a-FL to image sciatic nerves in vivo, mice were injected intravenously with Hs1a-FL alone (4 nmol, 45 μM of Hs1a-FL in 100 μL of PBS) or in combination with an excess of unmodified peptide (120 μM, 12 nmol in 100 PBS, block), and sacrificed 30 min after injection. Nerves were surgically harvested and flash-frozen in OCT blocks. Blocks were then sliced on a cryotome at a 10 μm thickness and imaged. Nerves were imaged to detect fluorescent signal and H&E stained to enable the visualization the Schwann cells within the nerve structure. Anti-Naᵥ1.7 immunohistochemistry was used to confirm target availability (FIG. 22B), and confocal microscopy confirmed the presence of Hs1a-FL signal in injected mice. No signal was detected in PBS-injected mice or blocked mice (FIG. 22B). In addition, no staining was observed when using isotype control antibodies, confirming specificity.

Ex Vivo Hs1a-FL Biodistribution

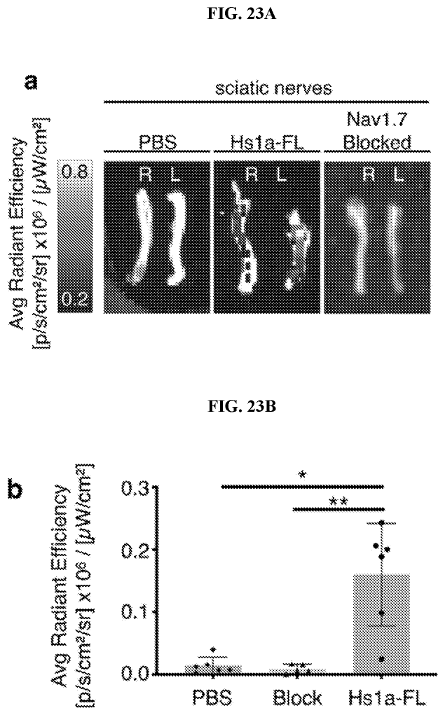

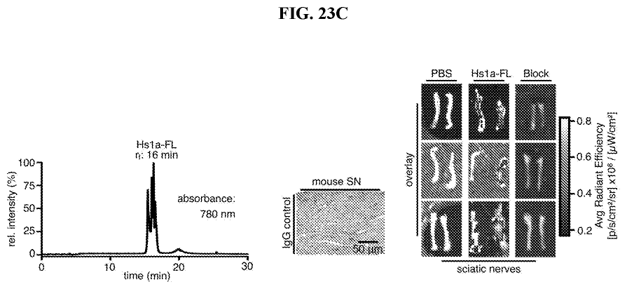

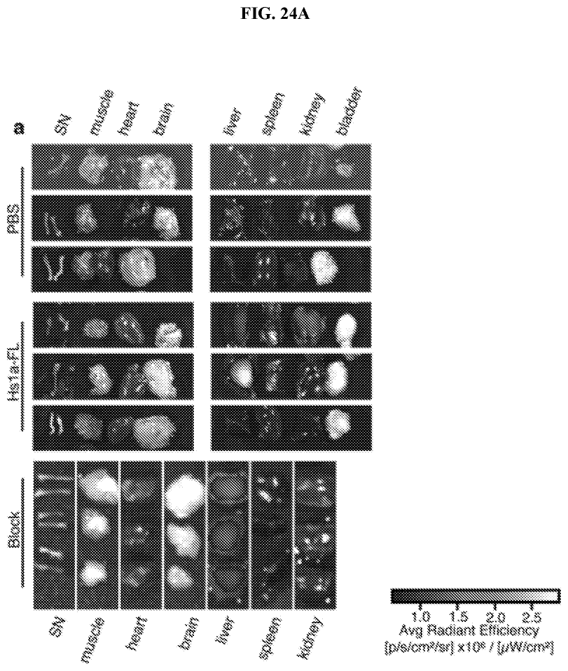

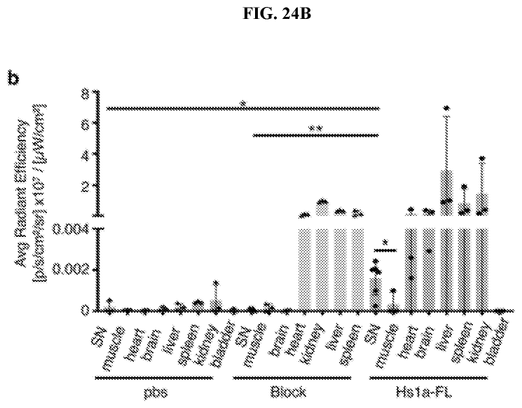

To determine the biodistribution of Hs1a-FL, mice were injected intravenously with Hs1a-FL alone (4 nmol, 45 μM of Hs1a-FL in 100 μL of PBS) or in combination with an excess of unmodified peptide (120 μM, 12 nmol in 100 μL PBS, block) and sacrificed 30 min after injection. The right and left sciatic nerves (RSN and LSN) were resected and epifluorescence imaging performed using an IVIS Spectrum in vivo imaging system (excitation=710/45 nm, emission=800-820 nm). In mice receiving just the imaging agent, accumulation of Hs1a-FL was observed in the resected sciatic nerves, which were visible (FIG. 23A, C). However, uptake was significantly reduced in the sciatic nerves of mice that received the imaging agent in combination with excess unmodified peptide. The radiant efficiency for Hs1a-FL was 1.6±0.3×10⁵ and that of the co-injection (blocking) was 0.09±0.03×10⁵ (Student's t-test, p-value<0.001, FIG. 23B). A trend towards higher fluorescence signals in the liver, kidney, brain and spleen was also observed (radiant efficiency: 3.0±2.0×10⁷ and 0.002±0.001×10⁷, 1.4±1.1×10⁷ and 0.005±0.004×10⁷, 0.2±0.1×10⁷ and 0.001±0.0005×10⁷ and 0.8±0.5×10⁷ and 0.004±0.0003×10⁷ for organs injected with fluorescent agent and with PBS, respectively; FIG. 24A-B).

Hs1a-FL is novel nerve-targeting agent would assist surgeons to identify peripheral nerves during surgical procedures and avoid surgical morbidity due to nerve injury.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A compound of a fluorophore conjugated to a side chain of an amino acid of a peptide of SEQ ID NO: 1, or a conservative amino acid substitution variant thereof, a pharmaceutically acceptable salt thereof, and/or a solvate thereof.
B. The compound of Paragraph A, wherein the compound is of Formula I

(I)
(SEQ ID NO: 3)
YCQK(α¹)FLWTCDSERPCCEGLVCRLWCK(α²)IN-NH₂,

or a conservative amino acid substitution variant thereof, a pharmaceutically acceptable salt thereof, and/or a solvate thereof,
wherein at least one of α¹ and α² is a fluorophore conjugated to the side chain amine of K and the remaining one of α¹ and α² is H.
C. The compound of Paragraph A or Paragraph B, wherein the compound of Formula I is of Formula IA

(IA)
(SEQ ID NO: 4)
YCQK(α¹)FLWTCDSERPCCEGLVCRLWCKIN-NH₂,

or a conservative amino acid substitution variant thereof, a pharmaceutically acceptable salt thereof, and/or a solvate thereof,
wherein X¹ is a fluorophore conjugated to the side chain amine of K.
D. The compound of any one of Paragraphs A-C, wherein the fluorophore independently at each occurrence arises from IR780, IR800, IR780, DY-684, DY-700, Janelia669, BODIPY, BODIPY665, sulfo-CY5, CY5.5, CY7, CY7.5, ICG, IR780, IR140, or DiR.
E. The compound of any one of Paragraphs A-D, wherein the fluorophore is independently at each occurrence selected from

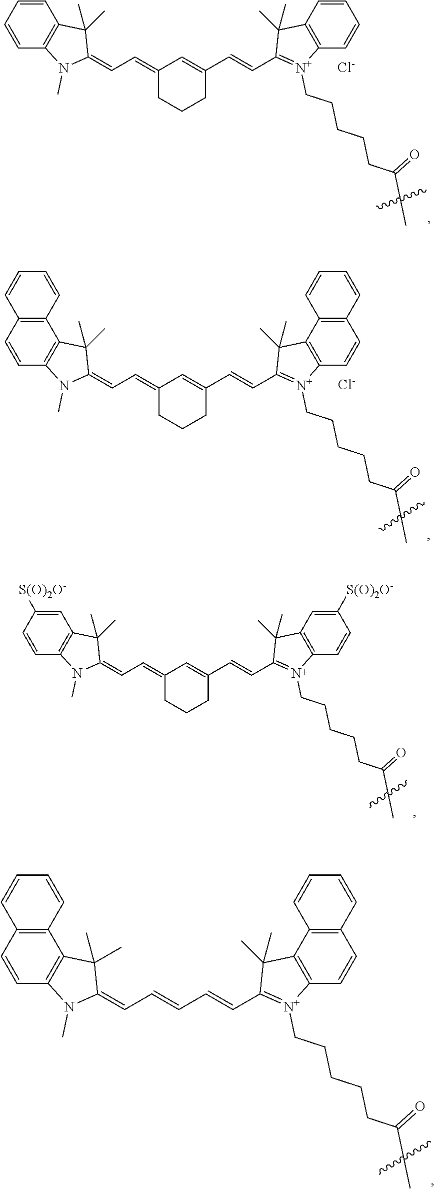

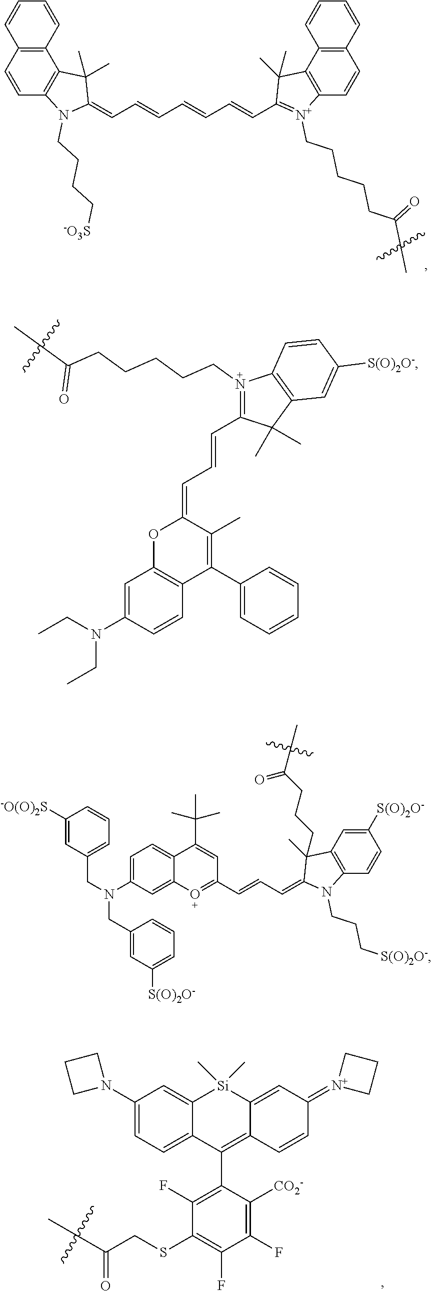

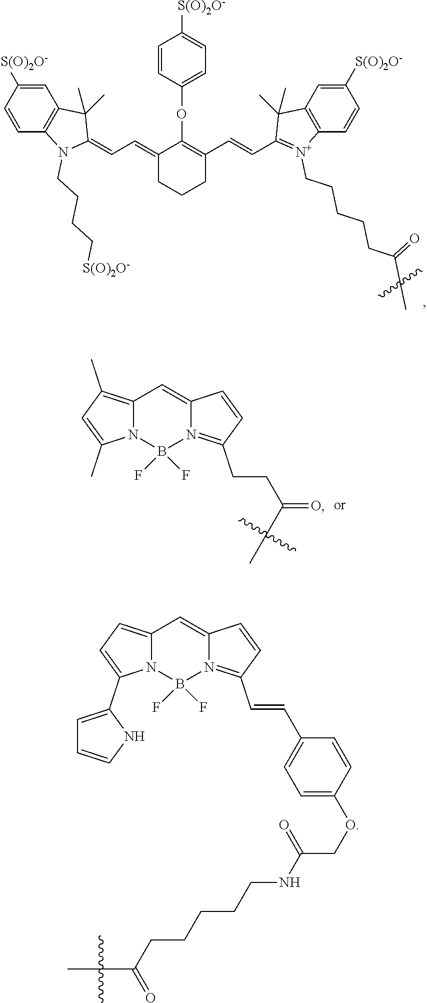

F. The compound of any one of Paragraphs A-E, wherein the compound is

(SEQ ID NO: 5)
YCQK(BODIPY)FLWTCDSERPCCEGLVCRLWCKIN-NH₂,
(SEQ ID NO: 6)
YCQK(IR-800)FLWTCDSERPCCEGLVCRLWCKIN-NH₂,
(SEQ ID NO: 7)
YCQK(DY-684)FLWTCDSERPCCEGLVCRLWCKIN-NH₂,
(SEQ ID NO: 8)
YCQK(Jane1ia669)FLWTCDSERPCCEGLVCRLWCKIN-NH₂,
(SEQ ID NO: 9)
YCQK(BODIPY665)FLWTCDSERPCCEGLVCRLWCKIN-NH₂,
(SEQ ID NO: 10)
YCQK(CY7.5)FLWTCDSERPCCEGLVCRLWCKIN-NH₂,

or a conservative amino acid substitution variant thereof, a pharmaceutically acceptable salt thereof, and/or a solvate thereof.
G. The compound of any one of Paragraphs C-E, wherein the compound is of Formula IA where α¹ is

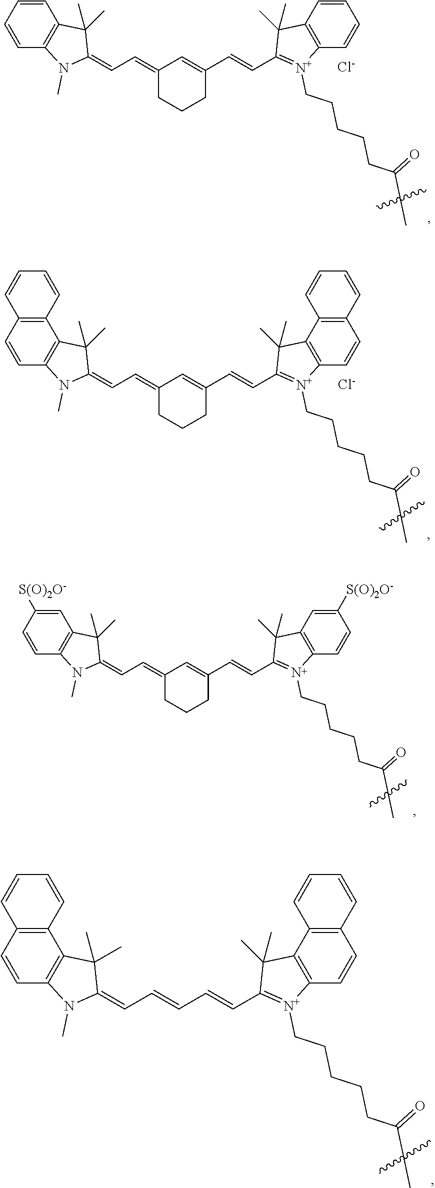

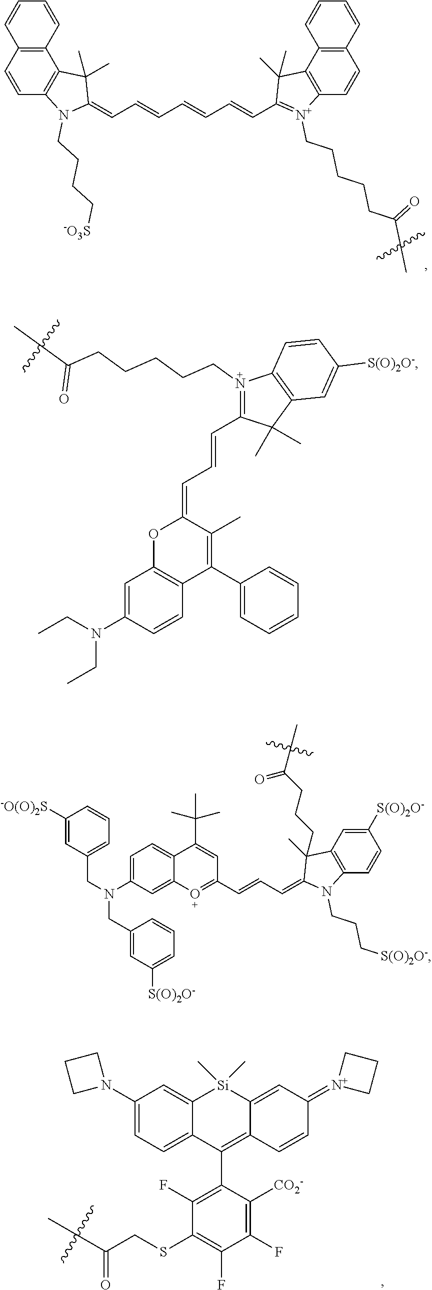

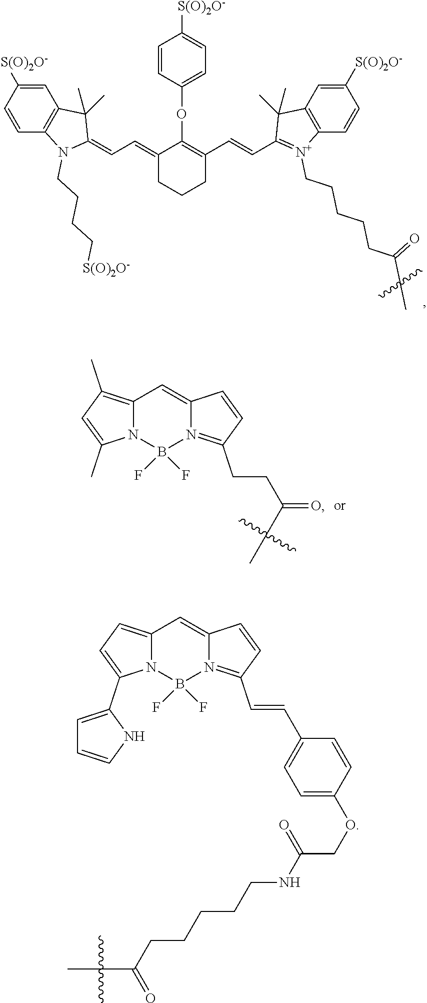

H. A composition comprising the compound of any one of Paragraphs A-F and a pharmaceutically acceptable carrier.
I. A pharmaceutical composition comprising an effective amount of the compound of any one of Paragraphs A-F for imaging peripheral neurons in a subject, and a pharmaceutically acceptable carrier.
J. A method comprising
   administering a compound of any one of Paragraphs A-F to a subject; and
   subsequent to the administering, detecting fluorescence emission.
K. The method of Paragraph J, wherein the method comprises administering an imaging-effective amount of the compound to the subject for imaging peripheral neurons.
L. The method of Paragraph J or Paragraph K, wherein the detecting comprises widefield intraoperative imaging, mesoscopic intraoperative imaging, microscopic intraoperative imaging, laparoscopic intraoperative imaging, or a combination of any two or more thereof.
M. The method of any one of Paragraphs J-L, wherein administering the compound comprises parenteral administration.
N. A method of obtaining an image, the method comprising
   administering an imaging-effective amount of a compound of any one of Paragraphs A-F for imaging peripheral neurons to a subject; and
   subsequent to the administering, detecting fluorescence emission.
O. The method of Paragraph N, wherein the detecting comprises widefield intraoperative imaging, mesoscopic intraoperative imaging, microscopic intraoperative imaging, laparoscopic intraoperative imaging, or a combination of any two or more thereof.
P. The method of Paragraph N or Paragraph O, wherein administering the compound comprises parenteral administration.
Q. A protein of SEQ ID NO: 1, or a conservative amino acid substitution variant thereof, a pharmaceutically acceptable salt thereof, and/or a solvate thereof.
R. A composition comprising the protein of Paragraph Q and a pharmaceutically acceptable carrier.
S. A pharmaceutical composition comprising an effective amount of the protein of Paragraph
Q for treating pain in a subject, and a pharmaceutically acceptable carrier.
T. The pharmaceutical composition of Paragraph S, wherein the subject is suffering from acute and/or chronic pain.
U. A method comprising administering an effective amount of a compound of a protein of Paragraph Q to a subject.
V. The method of Paragraph U, wherein the subject is suffering from acute and/or chronic pain.
W. A method comprising administering a pharmaceutical composition of Paragraph U or Paragraph V to a subject in need thereof.
X. The method of Paragraph W, wherein the subject is suffering from acute and/or chronic pain.
Y. A compound of a fluorophore conjugated to a side chain of an amino acid of a peptide of SEQ ID NO: 2, or a conservative amino acid substitution variant thereof, a pharmaceutically acceptable salt thereof, and/or a solvate thereof.
Z. The compound of Paragraph Y, wherein the compound is of Formula II

(II)
(SEQ ID NO: 11)
GNDCLGFWSACNPK(α³)NDK(α⁴)CCANLVCSSK(α⁵)HK(α⁶)WC
K(α⁷)GK(α⁸)L-NH₂

or a conservative amino acid substitution variant thereof, a pharmaceutically acceptable salt thereof, and/or a solvate thereof,
   wherein at least one of α³, α⁴, α⁵, α⁶, α⁷, and α⁸ is a fluorophore conjugated to the side chain amine of K and the remaining and the remaining of α³, α⁴, α⁵, α⁶, α⁷, and α⁸ are each H.
AA. The compound of Paragraph Z, wherein α³ is the fluorophore conjugated to the side chain amine of K and α⁴, α⁵, α⁶, α⁷, and α⁸ are each independently H.
AB. The compound of any one of Paragraphs Y-AA, wherein the fluorophore independently at each occurrence arises from IR780, IR800, IR780, DY-684, DY-700, Janelia669, BODIPY, BODIPY665, sulfo-CY5, CY5.5, CY7, CY7.5, ICG, IR780, IR140, or DiR
AC. The compound of any one of Paragraphs Z-AB, wherein the compound is of Formula II where α³ is

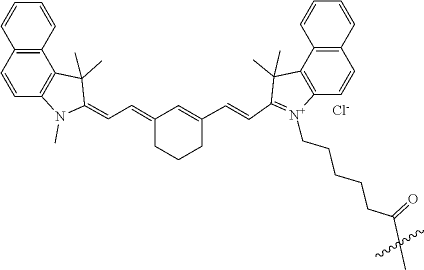 [SMILES: *C(=O)CCCCC[N+]1=C(/C=C/C2=CC(=C/C=C3/N(C)c4ccc5ccccc5c4C3(C)C)/CCC2)C(C)(C)c2c1ccc1ccccc21 ; [Cl-]]

and α⁴, α⁵, α⁶, α⁷, and α⁸ are each independently H, or a conservative amino acid substitution variant thereof, a pharmaceutically acceptable salt thereof, and/or a solvate thereof.
AD. A composition comprising the compound of any one of Paragraphs Y-AC and a pharmaceutically acceptable carrier.
AE. A pharmaceutical composition comprising an effective amount of the compound of any one of Paragraphs Y-AC for imaging peripheral neurons in a subject, and a pharmaceutically acceptable carrier.
AF. A method comprising
   administering a compound of any one of Paragraphs Y-AC to a subject; and
   subsequent to the administering, detecting fluorescence emission.
AG. The method of Paragraph AF, wherein the method comprises administering an imaging-effective amount of the compound to the subject for imaging peripheral neurons.
AH. The method of Paragraph AF or Paragraph AG, wherein the detecting comprises widefield intraoperative imaging, mesoscopic intraoperative imaging, microscopic intraoperative imaging, laparoscopic intraoperative imaging, or a combination of any two or more thereof.
AI. The method of any one of Paragraphs AF-AH, wherein administering the compound comprises parenteral administration.
AJ. A method of obtaining an image, the method comprising
   administering an imaging-effective amount of a compound of any one of Paragraphs Y-AC for imaging peripheral neurons to a subject; and
   subsequent to the administering, detecting fluorescence emission.
AK. The method of Paragraph AJ, wherein the detecting comprises widefield intraoperative imaging, mesoscopic intraoperative imaging, microscopic intraoperative imaging, laparoscopic intraoperative imaging, or a combination of any two or more thereof.
AL. The method of Paragraph AJ or Paragraph AK, wherein administering the compound comprises parenteral administration.

Other embodiments are set forth in the following claims.

1. A compound of a fluorophore conjugated to a side chain of an amino acid of a peptide of SEQ ID NO: 1, or a conservative amino acid substitution variant thereof, a pharmaceutically acceptable salt thereof, and/or a solvate thereof.

2. The compound of claim 1, wherein the compound is of Formula I
(I)
(SEQ ID NO: 3)
YCQK(α¹)FLWTCDSERPCCEGLVCRLWCK(α²)IN-NH₂,
or a conservative amino acid substitution variant thereof, a pharmaceutically acceptable salt thereof, and/or a solvate thereof,
wherein at least one of α¹ and α² is a fluorophore conjugated to the side chain amine of K and the remaining one of α¹ and α² is H.

3. The compound of claim 2, wherein the compound of Formula I is of Formula IA
(IA)
(SEQ ID NO: 4)
YCQK(α¹)FLWTCDSERPCCEGLVCRLWCKIN-NH₂,
or a conservative amino acid substitution variant thereof, a pharmaceutically acceptable salt thereof, and/or a solvate thereof.

4. The compound of claim 1, wherein the fluorophore independently at each occurrence arises from IR780, IR800, IR780, DY-684, DY-700, Janelia669, BODIPY, BODIPY665, sulfo-CY5, CY5.5, CY7, CY7.5, ICG, IR780, IR140, or DiR.

5. (canceled)

6. A composition comprising the compound of claim 1 and a pharmaceutically acceptable carrier.

7. A pharmaceutical composition comprising an effective amount of the compound of claim 1 for imaging peripheral neurons in a subject, and a pharmaceutically acceptable carrier.

8. A method comprising
administering a compound of claim 1 to a subject; and
subsequent to the administering, detecting fluorescence emission.

9.-11. (canceled)

12. A method of obtaining an image, the method comprising
administering an imaging-effective amount of a compound of claim 1 for imaging peripheral neurons to a subject; and
subsequent to the administering, detecting fluorescence emission.

13.-14. (canceled)

15. A protein of SEQ ID NO: 1, or a conservative amino acid substitution variant thereof, a pharmaceutically acceptable salt thereof, and/or a solvate thereof.

16. A composition comprising the protein of claim 15 and a pharmaceutically acceptable carrier.

17. A pharmaceutical composition comprising an effective amount of the protein of claim 15 for treating pain in a subject, and a pharmaceutically acceptable carrier.

18. (canceled)

19. A method comprising administering an effective amount of a compound of a protein of claim 15 to a subject.

20. (canceled)

21. A method comprising administering a pharmaceutical composition of claim 17 to a subject in need thereof.

22. (canceled)

23. A compound of a fluorophore conjugated to a side chain of an amino acid of a peptide of SEQ ID NO: 2 or a conservative amino acid substitution variant thereof, a pharmaceutically acceptable salt thereof, and/or a solvate thereof.

24. The compound of claim 23, wherein the compound is of Formula II
(II)
(SEQ ID NO: 11)
GNDCLGFWSACNPK(α³)NDK(α⁴)CCANLVCSSK(α⁵)HK(α⁶)WC
K(α⁷)GK(α⁸)L-NH₂
or a conservative amino acid substitution variant thereof, a pharmaceutically acceptable salt thereof, and/or a solvate thereof,
wherein at least one of α³, α⁴, α⁵, α⁶, α⁷, and α⁸ is a fluorophore conjugated to the side chain amine of K and the remaining of α³, α⁴, α⁵, α⁶, α⁷, and α⁸ are each H.

25.-26. (canceled)

27. The compound of claim 25, wherein α³ is

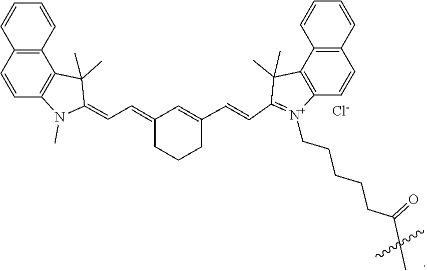 [SMILES: *C(=O)CCCCC[N+]1=C(/C=C/C2=CC(=C/C=C3/N(C)c4ccc5ccccc5c4C3(C)C)/CCC2)C(C)(C)c2c1ccc1ccccc21 ; [Cl-]]

28. A composition comprising the compound of claim 23 and a pharmaceutically acceptable carrier.

29. A pharmaceutical composition comprising an effective amount of the compound of claim 23 for imaging peripheral neurons in a subject, and a pharmaceutically acceptable carrier.

30. A method comprising
administering a compound of claim 23 to a subject; and
subsequent to the administering, detecting fluorescence emission.

31.-33. (canceled)

34. A method of obtaining an image, the method comprising
administering an imaging-effective amount of a compound of claim 23 for imaging peripheral neurons to a subject; and
subsequent to the administering, detecting fluorescence emission.

35.-36. (canceled)


SEQUENCE LISTING (32 sequences)

SEQ ID NO: 1 - protein, 28 aa, Unknown
  Description of Unknown Homoeomma spec. Peru spider venom sequence
YCQKFLWTCDSERPCCEGLVCRLWCKIN

SEQ ID NO: 2 - protein, 35 aa, Haplopelma schmidti
GNDCLGFWSACNPKNDKCCANLVCSSKHKWCKGKL

SEQ ID NO: 3 - protein, 28 aa, Artificial Sequence
  Description of Artificial Sequence Synthetic peptide
  MISC_FEATURE (4)..(4): May or may not be conjugated with a fluorophore on the side chain amine
  MISC_FEATURE (26)..(26): May or may not be conjugated with a fluorophore on the side chain amine
  See specification as filed for detailed description of substitutions and preferred embodiments
YCQKFLWTCDSERPCCEGLVCRLWCKIN

SEQ ID NO: 4 - protein, 28 aa, Artificial Sequence
  Description of Artificial Sequence Synthetic peptide
  MOD_RES (4)..(4): Lys conjugated with a fluorophore on the side chain amine
  See specification as filed for detailed description of substitutions and preferred embodiments
YCQKFLWTCDSERPCCEGLVCRLWCKIN

SEQ ID NO: 5 - protein, 28 aa, Artificial Sequence
  Description of Artificial Sequence Synthetic peptide
  MOD_RES (4)..(4): Lys(BODIPY)
YCQKFLWTCDSERPCCEGLVCRLWCKIN

SEQ ID NO: 6 - protein, 28 aa, Artificial Sequence
  Description of Artificial Sequence Synthetic peptide
  MOD_RES (4)..(4): Lys(IR-800)
YCQKFLWTCDSERPCCEGLVCRLWCKIN

SEQ ID NO: 7 - protein, 28 aa, Artificial Sequence
  Description of Artificial Sequence Synthetic peptide
  MOD_RES (4)..(4): Lys(DY-684)
YCQKFLWTCDSERPCCEGLVCRLWCKIN

SEQ ID NO: 8 - protein, 28 aa, Artificial Sequence
  Description of Artificial Sequence Synthetic peptide
  MOD_RES (4)..(4): Lys(Janelia669)
YCQKFLWTCDSERPCCEGLVCRLWCKIN

SEQ ID NO: 9 - protein, 28 aa, Artificial Sequence
  Description of Artificial Sequence Synthetic peptide
  MOD_RES (4)..(4): Lys(BODIPY665)
YCQKFLWTCDSERPCCEGLVCRLWCKIN

SEQ ID NO: 10 - protein, 28 aa, Artificial Sequence
  Description of Artificial Sequence Synthetic peptide
  MOD_RES (4)..(4): Lys(CY7.5)
YCQKFLWTCDSERPCCEGLVCRLWCKIN

SEQ ID NO: 11 - protein, 35 aa, Artificial Sequence; identical to SEQ ID NO: 2
  Description of Artificial Sequence Synthetic polypeptide
  MISC_FEATURE (14)..(14): May or may not be conjugated with a fluorophore on the side chain amine
  MISC_FEATURE (17)..(17): May or may not be conjugated with a fluorophore on the side chain amine
  MISC_FEATURE (27)..(27): May or may not be conjugated with a fluorophore on the side chain amine
  MISC_FEATURE (29)..(29): May or may not be conjugated with a fluorophore on the side chain amine
  MISC_FEATURE (32)..(32): May or may not be conjugated with a fluorophore on the side chain amine
  MISC_FEATURE (34)..(34): May or may not be conjugated with a fluorophore on the side chain amine
  See specification as filed for detailed description of substitutions and preferred embodiments

SEQ ID NO: 12 - protein, 6 aa, Artificial Sequence
  Description of Artificial Sequence Synthetic 6xHis tag
HHHHHH

SEQ ID NO: 13 - protein, 35 aa, Artificial Sequence; identical to SEQ ID NO: 2
  Description of Artificial Sequence Synthetic polypeptide
  MOD_RES (14)..(14): Lys(CY7.5)

SEQ ID NO: 14 - protein, 30 aa, Thrixopelma pruriens
YCQKWMWTCDSERKCCEGMVCRLWCKKKLW

SEQ ID NO: 15 - protein, 29 aa, Grammostola rosea
YCQKWLWTCDSERKCCEDMVCRLWCKKRL

SEQ ID NO: 16 - protein, 32 aa, Grammostola rosea
YCQKWMWTCDEERKCCEGLVCRLWCKKKIEEG

SEQ ID NO: 17 - protein, 31 aa, Grammostola rosea
YCQKWMWTCDEERKCCEGLVCRLWCKRIINM

SEQ ID NO: 18 - protein, 31 aa, Grammostola rosea
YCQKWMWTCDEERKCCEGLVCRLWCKKKIEW

SEQ ID NO: 19 - protein, 29 aa, Paraphysa scrofa
YCQKWMWTCDSARKCCEGLVCRLWCKKII

SEQ ID NO: 20 - protein, 30 aa, Grammostola rosea
YCQKWLWTCDSERKCCEDMVCRLWCKKRLG

SEQ ID NO: 21 - protein, 29 aa, Eucratoscelus constrictus
YCQKFLWTCDTERKCCEDMVCELWCKYKE

SEQ ID NO: 22 - protein, 30 aa, Chilobrachys guangxiensis
YCQKWMWTCDSERKCCEGYVCELWCKYNMG

SEQ ID NO: 23 - protein, 29 aa, Chilobrachys guangxiensis
YCQKWMWTCDSERKCCEGYVCELWCKYNL

SEQ ID NO: 24 - protein, 29 aa, Eucratoscelus constrictus
YCQKFLWTCDTERKCCEDMVCELWCKLEK

SEQ ID NO: 25 - protein, 30 aa, Grammostola rosea
YCQKWMWTCDSERKCCEDMVCELWCKKRLW

SEQ ID NO: 26 - protein, 29 aa, Pterinochilus murinus
YCQEFLWTCDEERKCCGDMVCRLWCKKRL

SEQ ID NO: 27 - protein, 29 aa, Eucratoscelus constrictus
YCQFKMWTCDSERKCCEDMVCRLWCKLNL

SEQ ID NO: 28 - protein, 30 aa, Grammostola rosea
YCQKWMWTCDSKRKCCEDMVCQLWCKKRLG

SEQ ID NO: 29 - protein, 29 aa, Grammostola rosea
YCQKWMWTCDSKRKCCEDMVCQLWCKKRL

SEQ ID NO: 30 - protein, 29 aa, Chilobrachys guangxiensis
YCQKWMWTCDSKRACCEGLRCKLWCRKII

SEQ ID NO: 31 - protein, 28 aa, Artificial Sequence
  Description of Artificial Sequence Synthetic peptide
YCQKFLWTCDSERPCCEGLVCRLWCKIN

SEQ ID NO: 32 - protein, 29 aa, Artificial Sequence
  Description of Artificial Sequence Synthetic peptide
SYCQKFLWTCDSERPCCEGLVCRLWCKIN